(19) United States
(12) Patent Application Publication
(10) Pub. No.: US 2005/0288295 A1
(43) Pub. Date: Dec. 29, 2005

(54) Certain imidazo[1,2-a]pyrazin-8-ylamines, method of making, and method of use thereof

(76) Inventors: Kevin S. Currie, North Branford, CT (US); Robert W. DeSimone, Durham, CT (US); Douglas A. Pippin, Branford, CT (US); James W. Darrow, Wallingford, CT (US); Scott A. Mitchell, East Haven, CT (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/985,023

(22) Filed: Nov. 10, 2004

Related U.S. Application Data
(60) Provisional application No. 60/519,311, filed on Nov. 11, 2003.

(51) Int. Cl. A61K 31/498; C07D 487/04

(52) U.S. Cl. 514/249; 544/349

(57) ABSTRACT

At least one chemical entity chosen from compounds of Formula 1 and pharmaceutically acceptable pharmaceutically acceptable salts, hydrates, solvates, crystal forms, diastereomers, and prodrugs thereof. 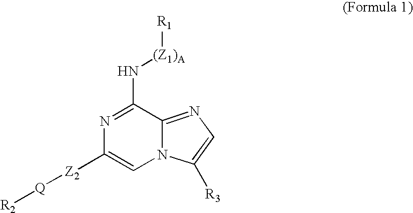 Methods of treating or preventing disorders in which aberrant kinase activity is implicated, pharmaceutically compositions, and methods for detecting the presence kinases in cells or biological preparations and for identifying kinases of therapeutic interest.

This application claims priority to U.S. provisional application 60/519,311 filed Nov. 11, 2003.

Provided herein are certain imidazo[1,2-a]pyrazinylamines and related compounds, compositions comprising such compounds, and methods of their use.

One of the central post-translational control elements in eukaryotic signal transduction is the phosphorylation of the hydroxyl moiety of serine, threonine, or tyrosine. The phosphorylation state of a given protein can govern its enzyme activity, stability, protein-protein binding interactions, and cellular distribution. Phosphorylation and dephosphorylation is thus a “chemical switch” that allows the cell to transmit signals from the plasma membrane to the nucleus, and to ultimately control gene expression. Kinases are involved in the control of cell metabolism, growth, differentiation, and apoptosis. Kinase signaling mechanisms have been implicated in the onset of cancer, metabolic disorders (for example diabetes), inflammation, immune system disorders, and neurodegeneration. Certain kinases have been implicated in cell proliferation and carcinogenesis. For example, many human cancers are caused by disregulation of a normal protein (e.g., when a proto-oncogene is converted to an oncogene through a gene translocation).

Inhibitors of kinases are among the most important pharmaceutical compounds known. Serine/threonine kinase inhibitors are also pharmaceutically important. For example, inhibitors of protein kinase C beta are known to be useful for treatment of diabetic macular edema and diabetic retinopathy. An inhibitor of cyclin-dependent kinases, is under development for treatment of mantle cell lymphoma (MCL) and fludar refractory chronic lymphocytic leukemia (CLL). One Raf kinase inhibitor is in development for treatment of solid tumors and myeloid leukemia, and another is being investigated for treatment of ovarian cancer. Several p38 mitogen-activated protein kinase inhibitors have been investigated for treatment of inflammation, rheumatoid arthritis, and myelodysplastic syndrome (MDS).

Modulators of kinase activity which may generally be described as imidazo[1,2-a]pyrazinylamines are provided herein. Certain compounds provided herein are inhibitors of angiogenic and/or oncogenic kinases.

In certain embodiment the invention provides at least one chemical entity chosen from compounds of Formula I: 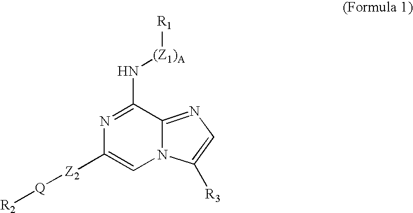
and pharmaceutically acceptable salts, solvates, crystal forms, diastereomers, and prodrugs thereof, wherein
A is chosen from 0 and 1;
Z₁ is chosen from 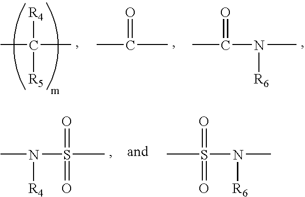
wherein
   each occurrence of R₄ and R₅ is independently chosen from hydrogen, C₁-C₆ alkyl, sulfonamido, and halo;
   m is chosen from 0, 1, 2, and 3; and
   R₆ is chosen from
      hydrogen,
      C₁-C₆ alkyl,
      phenyl,
      substituted phenyl chosen from mono-, di-, and tri-substituted phenyl wherein the substitutents are independently chosen from hydroxy, nitro, cyano, amino, halo, C₁-C₆ alkyl, C₁-C₆ haloalkyl, C₁-C₆ haloalkoxy, C₁-C₆ alkoxy, (C₁-C₆ alkoxy)C₁-C₆ alkoxy, mono-(C₁-C₆ alkyl)amino, di-(C₁-C₆ alkyl)amino, and amino(C₁-C₆ alkyl),
      heteroaryl, and
      substituted heteroaryl chosen from mono-, di-, and tri-substituted heteroaryl wherein the substitutents are independently chosen from hydroxy, nitro, cyano, amino, halo, C₁-C₆ alkyl, C₁-C₆ haloalkyl, C₁-C₆ haloalkoxy, C₁-C₆ alkoxy, (C₁-C₆ alkoxy)C₁-C₆ alkoxy, mono-(C₁-C₆ alkyl)amino, di-(C₁-C₆ alkyl)amino, and amino(C₁-C₆ alkyl);
R₁ is chosen from
   hydrogen,
   C₁-C₇ alkyl,
   C₁-C₆ alkoxy,
   C₃-C₇ cycloalkyl,
   (C₃-C₇ cycloalkyl)methyl,
   heterocycloalkyl,
   (heterocycloalkyl)C₁-C₂ alkyl,
   sulfonamido,
   (C₁-C₆ alkoxy)C₁-C₆ alkoxy,
   mono-(C₁-C₆ alkyl)amino,
   di-(C₁-C₆ alkyl)amino,
   mono-(C₁-C₆ alkyl)amino(C₁-C₆ alkyl),
   di(C₁-C₆ alkyl)amino(C₁-C₆ alkyl),
   phenyl,
   substituted phenyl chosen from mono-, di-, and tri-substituted phenyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
   phenyl fused to a 5- to 7-membered heterocycloalkyl ring containing 1 or 2 heteroatoms chosen from N, O, and S,
   substituted phenyl fused to a 5- to 7-membered heterocycloalkyl ring containing 1 or 2 heteroatoms chosen from N, O, and S, or heteroaryl, chosen from mono-, di-, and tri-substituted phenyl fused to a 5- to 7-membered heterocycloalkyl ring containing 1 or 2 heteroatoms chosen from N, O, and S, or heteroaryl, wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
   heteroaryl, and
   substituted heteroaryl chosen from mono-, di-, and tri-substituted heteroaryl, wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl;
Z₂ is chosen from
   phenylene,
   substituted phenylene chosen from mono-, di-, and tri-substituted phenylene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl),
   pyridylidene,
   substituted pyridylidene chosen from mono-, di-, and tri-substituted pyridylidene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl),
   naphthylidene, and
   substituted naphthylidene chosen from mono-, di-, and tri-substituted naphthylene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl);
Q is chosen from 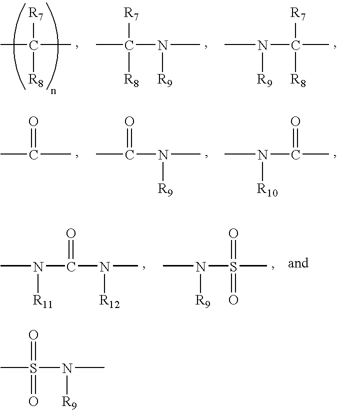
   wherein
      each occurrence of R₇ and R₈ is independently chosen from C₁-C₆ alkyl, sulfonamido, and halo;
      n is chosen from 0, 1, 2, and 3; and
      R₉-R₁₂ are each independently chosen from
         hydrogen,
         C₁-C₆ alkyl,
         phenyl,
         substituted phenyl chosen from mono-, di-, and tri-substituted phenyl wherein the substituents are independently chosen from hydroxy, nitro, carboxy, cyano, amino, halo, C₁-C₆ alkyl, C₁-C₆ alkoxy, (C₁-C₆ alkyloxy)C₁-C₆ alkoxy, C₁-C₆ perfluoroalkyl, C₁-C₆ perfluoroalkoxy, mono-(C₁-C₆ alkyl)amino, di(C₁-C₆ alkyl)amino, and amino(C₁-C₆ alkyl),
         heteroaryl, and
         substituted heteroaryl chosen from mono-, di-, and tri-substituted phenyl wherein the substituents are independently chosen from hydroxy, nitro, carboxy, cyano, amino, halo, C₁-C₆ alkyl, C₁-C₆ alkoxy, (C₁-C₆ alkyloxy)C₁-C₆ alkoxy, C₁-C₆ perfluoroalkyl, C₁-C₆ perfluoroalkoxy, mono-(C₁-C₆ alkyl)amino, di(C₁-C₆ alkyl)amino, and amino(C₁-C₆ alkyl);
R₂ is chosen from
   C₁-C₇ alkyl,
   substituted C₁-C₇ alkyl chosen from mono-, di-, and tri-substituted C₁-C₇ alkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   C₃-C₇ cycloalkyl,
   substituted C₃-C₇ cycloalkyl chosen from mono-, di-, and tri-substituted C₃-C₇ cycloalkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   heterocycloalkyl,
   substituted heterocycloalkyl chosen from mono-, di-, and tri-substituted heterocycloalkyl, wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   C₁-C₆ alkoxy,
   substituted C₁-C₆ alkoxy chosen from mono-, di-, and tri-substituted C₁-C₆ alkoxy, wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   aryloxy,
   substituted aryloxy chosen from mono-, di-, and tri-substituted aryloxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   phenyl,
   substituted phenyl chosen from mono-, di-, and tri-substituted phenyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   heteroaryl, and
   substituted heteroaryl chosen from mono-, di-, and tri-substituted heteroaryl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl; and
R₃ is chosen from hydrogen, C₁-C₇ alkyl, C₃-C₇ cycloalkyl, (C₃-C₇ cycloalkyl)C₁-C₂ alkyl, heterocycloalkyl, and (heterocycloalkyl)C₁-C₂ alkyl.

In certain embodiments, the invention provides a pharmaceutical composition comprising at least one chemical entity described herein, and at least one pharmaceutically acceptable carrier or excipient.

In certain embodiments, the invention provides methods of treating a kinase-implicated condition in a mammal having a kinase-implicated condition, comprising administering to the mammal a therapeutically effective amount of at least one chemical entity described herein.

In certain embodiments, the invention provides methods of treating cancer, comprising administering to a mammal in need thereof a therapeutically effective amount of at least one chemical entity described herein. In some embodiments, a therapeutically effective amount of at least one other antitumor therapeutic is also administered.

In certain embodiments, the invention provides methods for identifying a kinase, comprising contacting an organism, cell, or preparation comprising the kinase with at least one chemical entity described herein, and detecting modulation of an activity of the kinase.

In certain embodiments, the invention provides methods of treating a Btk-implicated condition in a mammal, comprising administering to the mammal a therapeutically effective amount of at least one chemical entity described herein.

In certain embodiments, the invention provides methods for identifying Btk, comprising contacting an organism, cell, or preparation comprising the kinase with at least one chemical entity described herein.

In certain embodiments, the invention provides methods of treating a Btk-implicated autoimmune/inflammatory condition in a mammal, comprising administering to the mammal a therapeutically effective amount of at least one chemical entity described herein.

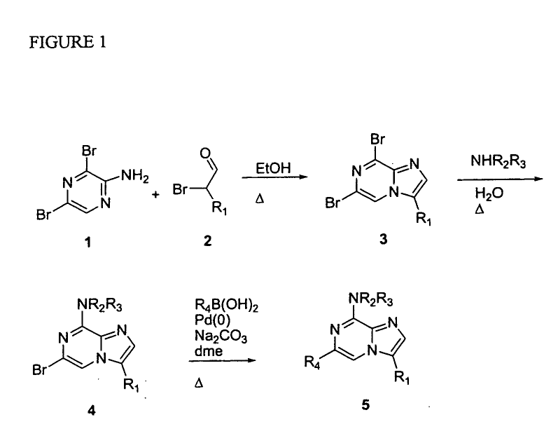

FIG. 1 is a schematic illustrating one synthesis of the present compounds.

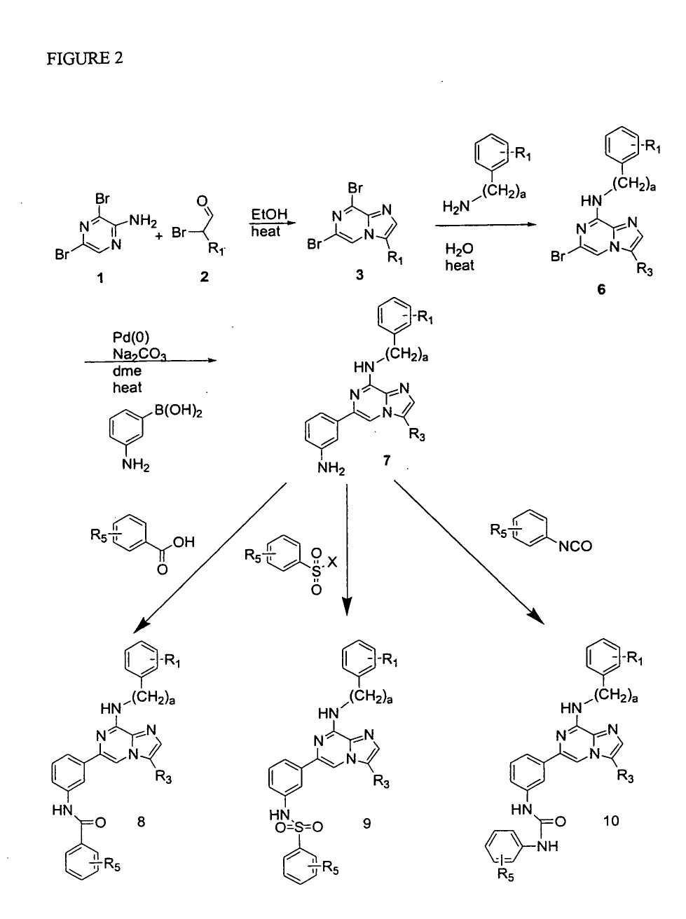

FIG. 2 is a schematic illustrating another synthesis of the present compounds.

As used in the present specification, the following words and phrases are generally intended to have the meanings as set forth below, except to the extent that the context in which they are used indicates otherwise. The following abbreviations and terms have the indicated meanings throughout:

As used herein, when any variable occurs more than one time in a chemical formula, its definition on each occurrence is independent of its definition at every other occurrence. In accordance with the usual meaning of “a” and “the” in patents, reference to “a” kinase or “the” kinase is inclusive of one or more kinases. Unless otherwise specified the terms “compound” and “compounds” include all pharmaceutically acceptable forms of the disclosed structures, salts, hydrates, solvates, prodrugs, and the like.

Formula I includes all subformulae described herein.

A dash (“-”) that is not between two letters or symbols is used to indicate a point of attachment for a substituent. For example, —CONH₂ is attached through the carbon atom.

By “optional” or “optionally” is meant that the subsequently described event or circumstance may or may not occur, and that the description includes instances where said event or circumstance occurs and instances in which it does not. For example, “optionally substituted alkyl” encompasses both “alkyl” and “substituted alkyl” as defined below. It will be understood by those skilled in the art, with respect to any group containing one or more substituents, that such groups are not intended to introduce any substitution or substitution patterns that are sterically impractical, synthetically non-feasible and/or inherently unstable.

“Alkyl” encompasses straight chain and branched chain having the indicated number of carbon atoms. For example C₁-C₆alkyl encompasses both straight and branched chain alkyl of from 1 to about 6 carbon atoms. Examples of alkyl groups include methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, and the like. Alkylene is another subset of alkyl, referring to the same residues as alkyl, but having two points of attachment. When an alkyl residue having a specific number of carbons is named, all geometric isomers having that number of carbons are intended to be encompassed; thus, for example, “butyl” is meant to include n-butyl, sec-butyl, isobutyl and t-butyl; “propyl” includes n-propyl and isopropyl.

“Cycloalkyl” indicates a saturated hydrocarbon ring group, having the specified number of carbon atoms, usually from 3 to about 7 ring carbon atoms. Examples of cycloalkyl groups include cyclopropyl, cyclobutyl, cyclopentyl, and cyclohexyl as well as bridged and caged saturated ring groups such as norbornane.

By “alkoxy” is meant an alkyl group of the indicated number of carbon atoms attached through an oxygen bridge such as, for example, methoxy, ethoxy, propoxy, isopropoxy, n-butoxy, sec-butoxy, tert-butoxy, pentoxy, 2-pentyloxy, isopentoxy, neopentoxy, hexoxy, 2-hexoxy, 3-hexoxy, 3-methylpentoxy, and the like. In certain embodiments, alkoxy groups herein are C₁-C₄alkoxy groups.

“Mono- and di-alkylcarboxamide” encompasses a group of the formula —(C═O)NRₐR_{b} where Rₐ and R_{b} are independently chosen from hydrogen and alkyl groups of the indicated number of carbon atoms, provided that Rₐ and R_{b} are not both hydrogen.

By “alkylthio” is meant an alkyl group of the indicated number of carbon atoms attached through a sulfur bridge.

“Alkanoyl” refers to an ester group of the formula —OC(O)(C₂-C₆ alkyl) attached through the ester oxygen.

By “alkoxycarbonyl” is meant an ester group of the formula (alkoxy)(C═O)— attached through the carbonyl carbon wherein the alkoxy group has the indicated number of carbon atoms. Thus a C₁-C₆alkoxycarbony group is an alkoxy group having from 1 to about 6 carbon atoms attached through its oxygen to a carbonyl linker.

By “amido” is meant —NH(C═O)R, wherein the R group is chosen from hydrogen and C₁-C₇alkyl. “Amido” also includes —(C═O)NRR, wherein each R is chosen from hydrogen and C₁-C₇alkyl. Except when R is hydrogen, each R may be unsubstituted or substituted with one or more, such as one, two or three, substituents independently chosen from halo, C₁-C₆alkyl, C₁-C₆haloalkyl, C₁-C₆haloalkoxy, C₁-C₆alkoxy, mono-(C₁-C₆ alkyl)amino, di-(C₁-C₆ alkyl)amino.

By “amino” is meant the group —NH₂.

“Mono- and di-(alkyl)amino” encompasses secondary and tertiary alkyl amino groups, wherein the alkyl groups are as defined above and have the indicated number of carbon atoms. The point of attachment of the alkylamino group is on the nitrogen. Examples of mono- and di-alkylamino groups include ethylamino, dimethylamino, and methyl-propyl-amino.

“Mono- and di-(alkyl)aminoalkyl” encompasses mono- and di-(alkyl)amino as defined above linked to an alkyl group.

By “amino(alkyl)” is meant an amino group linked to an alkyl group having the indicated number of carbons. Similarly “hydroxyalkyl” is a hydroxy group linked to an alkyl group.

“Aryl” encompasses:
5- and 6-membered carbocyclic aromatic rings, for example, benzene;
bicyclic 9- and 10-membered ring systems wherein at least one ring is carbocyclic and aromatic, for example, naphthalene, indane, and tetralin; and
tricyclic 12- to 14-membered ring systems wherein at least one ring is carbocyclic and aromatic, for example, fluorene.
   For example, aryl includes 5- and 6-membered carbocyclic aromatic rings fused to a 5- to 7-membered heterocycloalkyl ring containing 1 or more heteroatoms chosen from N, O, and S. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene.

The term “aryloxy” refers to the group —O-aryl.

The term “carboxy” refers to the group —C(O)OH.

The term “halo” includes fluoro, chloro, bromo, and iodo, and the term “halogen” includes fluorine, chlorine, bromine, and iodine.

“Haloalkyl” indicates alkyl as defined above having the specified number of carbon atoms, substituted with 1 or more halogen atoms, generally up to the maximum allowable number of halogen atoms. Examples of haloalkyl include, but are not limited to, trifluoromethyl, difluoromethyl, 2-fluoroethyl, and penta-fluoroethyl.

“Heteroaryl” encompasses:
5- to 7-membered aromatic, monocyclic rings containing one or more, for example, from 1 to 4, or in certain embodiments, from 1 to 3, heteroatoms chosen from N, O, and S, with the remaining ring atoms being carbon; and
7- to 10-membered bicyclic heterocyclic rings containing one or more, for example, from 1 to 4, or in certain embodiments, from 1 to 3, heteroatoms chosen from N, O, and S, with the remaining ring atoms being carbon and wherein at least one heteroatom is present in an aromatic ring.
   For example, heteroaryl includes 5- to 7-membered heterocycloalkyl, aromatic rings fused to a 5- to 7-membered cycloalkyl ring. For such fused, bicyclic heteroaryl ring systems wherein only one of the rings contains one or more heteroatoms, the point of attachment may be at the heteroaromatic ring or the cycloalkyl ring. When the total number of S and O atoms in the heteroaryl group exceeds 1, those heteroatoms are not adjacent to one another. In certain embodiments, the total number of S and O atoms in the heteroaryl group is not more than 2. In certain embodiments, the total number of S and O atoms in the aromatic heterocycle is not more than 1. Examples of heteroaryl groups include, but are not limited to, systems (as numbered from the linkage position assigned priority 1), such as 2-pyridyl, 3-pyridyl, 4-pyridyl, 2,3-pyrazinyl, 3,4-pyrazinyl, 2,4-pyrimidinyl, 3,5-pyrimidinyl, 2,3-pyrazolinyl, 2,4-imidazolinyl, isoxazolinyl, oxazolinyl, thiazolinyl, thiadiazolinyl, tetrazolyl, thienyl, benzothiophenyl, furanyl, benzofuranyl, benzoimidazolinyl, indolinyl, pyridizinyl, triazolyl, quinolinyl, pyrazolyl, and 5,6,7,8-tetrahydroisoquinoline. Bivalent radicals derived from univalent heteroaryl radicals whose names end in “-yl” by removal of one hydrogen atom from the atom with the free valence are named by adding “-idene” to the name of the corresponding univalent radical, e.g., a pyridyl group with two points of attachment is a pyridylidene.

In the term “heteroarylalkyl,” heteroaryl and alkyl are as defined herein, and the point of attachment is on the alkyl group. This term encompasses, but is not limited to, pyridylmethyl, thiophenylmethyl, and (pyrrolyl)1-ethyl.

By “heterocycloalkyl” is meant a single aliphatic ring containing at least 2 carbon atoms in addition to 1-3 heteroatoms independently selected from oxygen, sulfur, and nitrogen, and the like, as well as combinations comprising at least one of the foregoing heteroatoms. Suitable heterocycloalkyl groups include, for example (as numbered from the linkage position assigned priority 1), 2-pyrrolinyl, 2,4-imidazolidinyl, 2,3-pyrazolidinyl, 2-piperidyl, 3-piperidyl, 4-piperdyl, and 2,5-piperzinyl. Morpholinyl groups are also contemplated, including 2-morpholinyl and 3-morpholinyl (numbered wherein the oxygen is assigned priority 1).

As used herein, “modulation” refers to a change in kinase activity as a direct or indirect response to the presence of compounds of Formula 1, relative to the activity of the kinase in the absence of the compound. The change may be an increase in activity or a decrease in activity, and may be due to the direct interaction of the compound with the kinase, or due to the interaction of the compound with one or more other factors that in turn affect kinase activity. For example, the presence of the compound may, for example, increase or decrease kinase activity by directly binding to the kinase, by causing (directly or indirectly) another factor to increase or decrease the kinase activity, or by (directly or indirectly) increasing or decreasing the amount of kinase present in the cell or organism.

By “piperazinyl” is meant unsubstituted piperazine, as well as piperazines independently substituted on 1-4 carbon atoms with at least one substituent chosen from hydroxy, cyano, amino, halo, C₁-C₆ alkyl, C₁-C₆ perfluoroalkyl, C₁-C₆ perfluoroalkoxy, C₁-C₆ alkoxy, mono-(C₁-C₆ alkyl)amino, di(C₁-C₆ alkyl)amino; mono-(C₁-C₆ alkyl)amino(C₁-C₆ alkyl), di(C₁-C₆ alkyl)amino(C₁-C₆ alkyl), and sulfonamido.

By “sulfonamido” is meant —S(O)₂N— in either S-linked (—S(O)₂NRR) or N-linked orientation —NS(O)₂RR orientation, wherein each R may be independently chosen from hydrogen and C₁-C₇alkyl wherein alkyl is as defined above, such as 3- to 7-membered cycloalkyl, and heterocycloalkyl rings. When R is not hydrogen, each R may be unsubstituted or substituted with one or more, such as one, two or three, substituents independently chosen from, e.g., halo, C₁-C₆alkyl, C₁-C₆ haloalkyl, C₁-C₆ haloalkoxy, C₁-C₆alkoxy, mono-(C₁-C₆ alkyl)amino, and di-(C₁-C₆ alkyl)amino.

The term “substituted”, as used herein, means that any one or more hydrogens on the designated atom or group is replaced with a selection from the indicated group, provided that the designated atom's normal valence is not exceeded. When a substituent is oxo (i.e., ═O) then 2 hydrogens on the atom are replaced. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent. Unless otherwise specified, substituents are named into the core structure. For example, it is to be understood that when (cycloalkyl)alkyl is listed as a possible substituent, the point of attachment of this substituent to the core structure is in the alkyl portion.

If the compounds of Formula I have asymmetric centers, then Formula I includes all of the optical isomers and mixtures thereof. In addition, compounds with carbon-carbon double bonds may occur in Z- and E-forms, with all isomeric forms of the compounds being included. Those compounds can be, for example, racemates or optically active forms. In those situations, the single enantiomers or diastereomers, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-pressure liquid chromatography (HPLC) column. Where compounds of Formula I exists in various tautomeric forms, the invention is not limited to any one of the specific tautomers, and includes all tautomeric forms of the compound.

Chemical entities of the present invention include, but are not limited to, compounds of Formula I and all pharmaceutically acceptable forms thereof. Pharmaceutically acceptable forms of the compounds recited herein include pharmaceutically acceptable salts, hydrates, solvates, crystal forms, polymorphs, chelates, non-covalent complexes, esters, clathrates, prodrugs, and mixtures of such compounds. In certain embodiments, the compounds described herein are in the form of pharmaceutically acceptable salts. Hence, the terms “chemical entity” and “chemical entities” also encompass pharmaceutically acceptable salts, hydrates, solvates, crystal forms, polymorphs, chelates, non-covalent complexes, esters, clathrates, prodrugs, and mixtures of such compounds.

“Pharmaceutically acceptable salts” include, but are not limited to salts with inorganic acids, such as hydrochlorate, phosphate, diphosphate, hydrobromate, sulfate, sulfinate, nitrate, and like salts; as well as salts with an organic acid, such as malate, maleate, fumarate, tartrate, succinate, citrate, acetate, lactate, methanesulfonate, p-toluenesulfonate, 2-hydroxyethylsulfonate, benzoate, salicylate, stearate, and alkanoate such as acetate, HOOC—(CH₂)ₙ—COOH where n is 0-4, and like salts. Similarly, pharmaceutically acceptable cations include, but are not limited to sodium, potassium, calcium, aluminum, lithium, and ammonium.

In addition, if the compound of Formula I is obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts encompassed by Formula I.

As noted above, prodrugs also fall within the scope of chemical entities, for example acylated prodrugs of the compounds of Formula I. The term “prodrugs” includes any compounds that become compounds of Formula I when administered to a patient, e.g., upon metabolic processing of the prodrug. Examples of prodrugs include, but are not limited to, acetate, formate, and benzoate and like derivatives of functional groups (such as alcohol or amine groups) in the compounds of Formula I.

The term “solvate” refers to the compound formed by the interaction of a solvent and a compound. Suitable solvates are pharmaceutically acceptable solvates, such as hydrates, including monohydrates and hemi-hydrates.

The term “active agent” is used to indicate a chemical entity which has biological activity. In certain embodiments, an “active agent” is a compound having pharmaceutical utility. For example an active agent may be an anti-cancer therapeutic.

“Treatment or treating means any treatment of a disease in a patient, including:
a) preventing the disease, that is, causing the clinical symptoms of the disease not to develop;
b) inhibiting the disease;
c) slowing or arresting the development of clinical symptoms; and/or
d) relieving the disease, that is, causing the regression of clinical symptoms.

“Diseases or disorders responsive to kinase modulation” refer to pathologic conditions that depend, at least in part, on the activity of one or more protein kinases, for example, angiogenic kinases and/or oncogenic kinases. Kinases either directly or indirectly participate in the signal transduction pathways of a variety of cellular activities including cell proliferation, differentiation, and invasion. Diseases or disorders responsive to kinase modulation include but are not limited to psoriasis, cancer (for example, chronic myelogenous leukemia, gastrointestinal stromal tumors, non-small cell lung cancer, breast cancer, ovarian cancer, recurrent ovarian cancer, prostate cancer such as hormonal refractory prostate cancer, kidney cancer, head and neck cancer, or colorectal cancer), immunoregulation (graft rejection), atherosclerosis, rheumatoid arthritis, Parkinson's disease, Alzheimer's disease, diabetes (for example insulin resistance or diabetic retinopathy), septic shock, and the like.

“Patient” refers to an animal, such as a mammal, for example a human, that has been or will be the object of treatment, observation or experiment. The methods of the invention can be useful in both human therapy and veterinary applications. In some embodiments, the patient is a mammal, and in some embodiments the patient is human.

The term “therapeutically effective amount” of a chemical entity of this invention means an amount effective, when administered to a human or non-human patient, to provide a therapeutic benefit such as an amelioration of symptoms, e.g., an amount effective to at least decrease the symptoms of a disease or disorder responsive to kinase modulation, including those diseases and disorders response to modulation of ephrin receptors, such as ephrin B receptors, and including EphB4, and, in certain embodiments, an amount sufficient to reduce cancer symptoms, decrease the number of detectable cancerous cells in an organism, detectably slow or stop the growth of a cancerous tumor, or, in certain embodiments, to shrink a cancerous tumor. In certain circumstances a patient suffering from cancer may not present symptoms of being affected. Thus a therapeutically effective amount of a compound is also an amount sufficient to prevent a significant increase or significantly reduce the detectable level of cancerous cells or cancer markers in the patient's blood, serum, or tissues. A significant increase or reduction in the detectable level of cancerous cells or cancer markers is any detectable change that is statistically significant in a standard parametric test of statistical significance such as Student's T-test, where p<0.05.

Within certain embodiments, the invention provides at least one chemical entity chosen from compounds of Formula I: 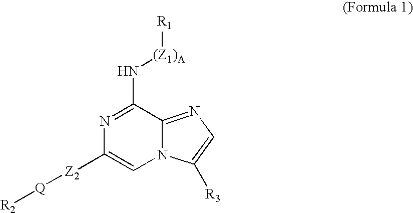
and pharmaceutically acceptable salts, solvates, crystal forms, diastereomers, and prodrugs thereof, wherein
A is chosen from 0 and 1;
Z₁ is chosen from 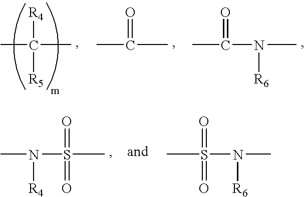
wherein
   each occurrence of R₄ and R₅ is independently chosen from hydrogen, C₁-C₆ alkyl, sulfonamido, and halo, and m is chosen from 0, 1, 2, and 3;
   R₆ is chosen from
      hydrogen,
      C₁-C₆ alkyl,
      phenyl,
      substituted phenyl chosen from mono-, di-, and tri-substituted phenyl wherein the substitutents are independently chosen from hydroxy, nitro, cyano, amino, halo, C₁-C₆ alkyl, C₁-C₆ haloalkyl, C₁-C₆ haloalkoxy, C₁-C₆ alkoxy, (C₁-C₆ alkoxy)C₁-C₆ alkoxy, mono-(C₁-C₆ alkyl)amino, di-(C₁-C₆ alkyl)amino, and amino(C₁-C₆ alkyl),
      heteroaryl, and
      substituted heteroaryl chosen from mono-, di-, and tri-substituted heteroaryl wherein the substitutents are independently chosen from hydroxy, nitro, cyano, amino, halo, C₁-C₆ alkyl, C₁-C₆ haloalkyl, C₁-C₆ haloalkoxy, C₁-C₆ alkoxy, (C₁-C₆ alkoxy)C₁-C₆ alkoxy, mono-(C₁-C₆ alkyl)amino, di-(C₁-C₆ alkyl)amino, and amino(C₁-C₆ alkyl);
R₁ is chosen from
   hydrogen,
   C₁-C₇ alkyl,
   C₁-C₆ alkoxy,
   C₃-C₇ cycloalkyl,
   (C₃-C₇ cycloalkyl)methyl,
   heterocycloalkyl,
   (heterocycloalkyl)C₁-C₂ alkyl,
   sulfonamido,
   (C₁-C₆ alkoxy)C₁-C₆ alkoxy,
   mono-(C₁-C₆ alkyl)amino,
   di-(C₁-C₆ alkyl)amino,
   mono-(C₁-C₆ alkyl)amino(C₁-C₆ alkyl),
   di(C₁-C₆ alkyl)amino(C₁-C₆ alkyl),
   phenyl,
   substituted phenyl chosen from mono-, di-, and tri-substituted phenyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
   phenyl fused to a 5- to 7-membered heterocycloalkyl ring containing 1 or 2 heteroatoms chosen from N, O, and S,
   substituted phenyl fused to a 5- to 7-membered heterocycloalkyl ring containing 1 or 2 heteroatoms chosen from N, O, and S, or heteroaryl, chosen from mono-, di-, and tri-substituted phenyl fused to a 5- to 7-membered heterocycloalkyl ring containing 1 or 2 heteroatoms chosen from N, O, and S, or heteroaryl, wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
   heteroaryl, and
   substituted heteroaryl chosen from mono-, di-, and tri-substituted heteroaryl, wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl;
Z₂ is chosen from
   phenylene,
   substituted phenylene chosen from mono-, di-, and tri-substituted phenylene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl),
   pyridylidene,
   substituted pyridylidene chosen from mono-, di-, and tri-substituted pyridylidene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl),
   naphthylidene, and
   substituted naphthylidene chosen from mono-, di-, and tri-substituted naphthylene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl);
Q is chosen from 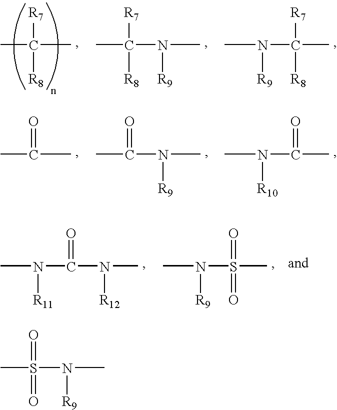
   wherein
      each occurrence of R₇ and R₈ is independently chosen from C₁-C₆ alkyl, sulfonamido, and halo; and n is chosen from 0, 1, 2, and 3; and
      R₉-R₁₂ are each independently chosen from
         hydrogen,
         C₁-C₆ alkyl,
         phenyl,
         substituted phenyl chosen from mono-, di-, and tri-substituted phenyl wherein the substituents are independently chosen from hydroxy, nitro, cyano, carboxy, amino, halo, C₁-C₆ alkyl, C₁-C₆ alkoxy, (C₁-C₆ alkyloxy)C₁-C₆ alkoxy, C₁-C₆ perfluoroalkyl, C₁-C₆ perfluoroalkoxy, mono-(C₁-C₆ alkyl)amino, di(C₁-C₆ alkyl)amino, and amino(C₁-C₆ alkyl),
         heteroaryl, and
         substituted heteroaryl chosen from mono-, di-, and tri-substituted heteroaryl wherein the substituents are independently chosen from hydroxy, nitro, cyano, carboxy, amino, halo, C₁-C₆ alkyl, C₁-C₆ alkoxy, (C₁-C₆ alkyloxy)C₁-C₆ alkoxy, C₁-C₆ perfluoroalkyl, C₁-C₆ perfluoroalkoxy, mono-(C₁-C₆ alkyl)amino, di(C₁-C₆ alkyl)amino, and amino(C₁-C₆ alkyl);
R₂ is chosen from
   C₁-C₇ alkyl,
   substituted C₁-C₇ alkyl chosen from mono-, di-, and tri-substituted C₁-C₇ alkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   C₃-C₇ cycloalkyl,
   substituted C₃-C₇ cycloalkyl chosen from mono-, di-, and tri-substituted C₃-C₇ cycloalkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   heterocycloalkyl,
   substituted heterocycloalkyl chosen from mono-, di-, and tri-substituted heterocycloalkyl, wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   C₁-C₆ alkoxy,
   substituted C₁-C₆ alkoxy chosen from mono-, di-, and tri-substituted C₁-C₆ alkoxy, wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   aryloxy,
   substituted aryloxy chosen from mono-, di-, and tri-substituted aryloxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   phenyl,
   substituted phenyl chosen from mono-, di-, and tri-substituted phenyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁₋C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   heteroaryl, and
   substituted heteroaryl chosen from mono-, di-, and tri-substituted heteroaryl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl; and
R₃ is chosen from hydrogen, C₁-C₇ alkyl, C₃-C₇ cycloalkyl, (C₃-C₇ cycloalkyl)C₁-C₂ alkyl, heterocycloalkyl, and (heterocycloalkyl)C₁-C₂ alkyl.

Within certain embodiments, the invention provides at least one chemical entity chosen from compounds of Formula I: 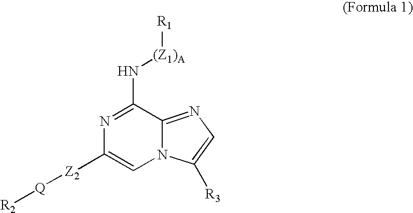
and pharmaceutically acceptable salts, solvates, crystal forms, diastereomers, and prodrugs thereof, wherein
A is chosen from 0 and 1;
Z₁ is chosen from 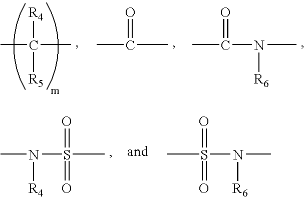
wherein
   each occurrence of R₄ and R₅ is independently chosen from hydrogen, C₁-C₆ alkyl, sulfonamido, and halo, and m is chosen from 0, 1, 2, and 3;
   R₆ is chosen from
      hydrogen,
      C₁-C₆ alkyl,
      phenyl,
      substituted phenyl chosen from mono-, di-, and tri-substituted phenyl wherein the substitutents are independently chosen from hydroxy, nitro, cyano, amino, halo, C₁-C₆ alkyl, C₁-C₆ haloalkyl, C₁-C₆ haloalkoxy, C₁-C₆ alkoxy, (C₁-C₆ alkoxy)C₁-C₆ alkoxy, mono-(C₁-C₆ alkyl)amino, di-(C₁-C₆ alkyl)amino, and amino(C₁-C₆ alkyl),
      heteroaryl, and
      substituted heteroaryl chosen from mono-, di-, and tri-substituted heteroaryl wherein the substitutents are independently chosen from hydroxy, nitro, cyano, amino, halo, C₁-C₆ alkyl, C₁-C₆ haloalkyl, C₁-C₆ haloalkoxy, C₁-C₆ alkoxy, (C₁-C₆ alkoxy)C₁-C₆ alkoxy, mono-(C₁-C₆ alkyl)amino, di-(C₁-C₆ alkyl)amino, and amino(C₁-C₆ alkyl);
R₁ is chosen from
   hydrogen,
   C₁-C₇ alkyl,
   C₁-C₆ alkoxy,
   C₃-C₇ cycloalkyl,
   (C₃-C₇ cycloalkyl)methyl,
   heterocycloalkyl,
   (heterocycloalkyl)C₁-C₂ alkyl,
   sulfonamido,
   (C₁-C₆ alkoxy)C₁-C₆ alkoxy,
   mono-(C₁-C₆ alkyl)amino,
   di-(C₁-C₆ alkyl)amino,
   mono-(C₁-C₆ alkyl)amino(C₁-C₆ alkyl),
   di(C₁-C₆ alkyl)amino(C₁-C₆ alkyl),
   phenyl,
   substituted phenyl chosen from mono-, di-, and tri-substituted phenyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
   phenyl fused to a 5- to 7-membered heterocycloalkyl ring containing 1 or 2 heteroatoms chosen from N, O, and S,
   substituted phenyl fused to a 5- to 7-membered heterocycloalkyl ring containing 1 or 2 heteroatoms chosen from N, O, and S, or heteroaryl, chosen from mono-, di-, and tri-substituted phenyl fused to a 5- to 7-membered heterocycloalkyl ring containing 1 or 2 heteroatoms chosen from N, O, and S, or heteroaryl, wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
   heteroaryl, and
   substituted heteroaryl chosen from mono-, di-, and tri-substituted heteroaryl, wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl;
Z₂ is chosen from
   substituted phenylene chosen from mono-, di-, and tri-substituted phenylene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl),
   pyridylidene,
   substituted pyridylidene chosen from mono-, di-, and tri-substituted pyridylidene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl), and
   substituted naphthylidene chosen from mono-, di-, and tri-substituted naphthylene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl);
Q is chosen from 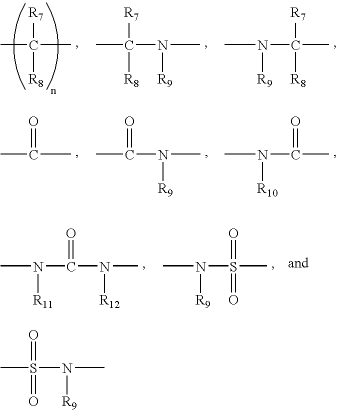
   wherein
      each occurrence of R₇ and R₈ is independently chosen from C₁-C₆ alkyl, sulfonamido, and halo; and n is chosen from 0, 1, 2, and 3; and
      R₉-R₁₂ are each independently chosen from
         hydrogen,
         C₁-C₆ alkyl,
         phenyl,
         substituted phenyl chosen from mono-, di-, and tri-substituted phenyl wherein the substituents are independently chosen from hydroxy, nitro, cyano, amino, halo, C₁-C₆ alkyl, C₁-C₆ alkoxy, (C₁-C₆ alkyloxy)C₁-C₆ alkoxy, C₁-C₆ perfluoroalkyl, C₁-C₆ perfluoroalkoxy, mono-(C₁-C₆ alkyl)amino, di(C₁-C₆ alkyl)amino, and amino(C₁-C₆ alkyl),
         heteroaryl, and
         substituted heteroaryl chosen from mono-, di-, and tri-substituted heteroaryl wherein the substituents are independently chosen from hydroxy, nitro, cyano, amino, halo, C₁-C₆ alkyl, C₁-C₆ alkoxy, (C₁-C₆ alkyloxy)C₁-C₆ alkoxy, C₁-C₆ perfluoroalkyl, C₁-C₆ perfluoroalkoxy, mono-(C₁-C₆ alkyl)amino, di(C₁-C₆ alkyl)amino, and amino(C₁-C₆ alkyl);
R₂ is chosen from
   C₁-C₇ alkyl,
   substituted C₁-C₇ alkyl chosen from mono-, di-, and tri-substituted C₁-C₇ alkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   C₃-C₇ cycloalkyl,
   substituted C₃-C₇ cycloalkyl chosen from mono-, di-, and tri-substituted C₃-C₇ cycloalkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   heterocycloalkyl,
   substituted heterocycloalkyl chosen from mono-, di-, and tri-substituted heterocycloalkyl, wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   C₁-C₆ alkoxy,
   substituted C₁-C₆ alkoxy chosen from mono-, di-, and tri-substituted C₁-C₆ alkoxy, wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   aryloxy,
   substituted aryloxy chosen from mono-, di-, and tri-substituted aryloxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   phenyl,
   substituted phenyl chosen from mono-, di-, and tri-substituted phenyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   heteroaryl, and
   substituted heteroaryl chosen from mono-, di-, and tri-substituted heteroaryl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl; and
R₃ is chosen from hydrogen, C₁-C₇ alkyl, C₃-C₇ cycloalkyl, (C₃-C₇ cycloalkyl)C₁-C₂ alkyl, heterocycloalkyl, and (heterocycloalkyl)C₁-C₂ alkyl.

Within certain embodiments, the invention provides at least one chemical entity chosen from compounds of Formula I: 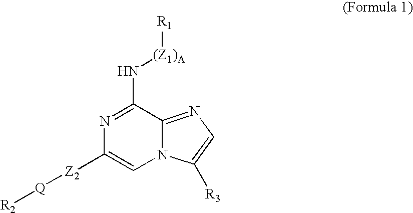
and pharmaceutically acceptable salts, solvates, crystal forms, diastereomers, and prodrugs thereof, wherein
A is chosen from 0 and 1;
Z₁ is chosen from 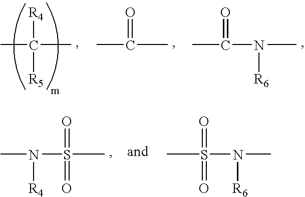
wherein
   each occurrence of R₄ and R₅ is independently chosen from hydrogen, C₁-C₆ alkyl, sulfonamido, and halo, and m is chosen from 0, 1, 2, and 3;
   R₆ is chosen from
      hydrogen,
      C₁-C₆ alkyl,
      phenyl,
      substituted phenyl chosen from mono-, di-, and tri-substituted phenyl wherein the substitutents are independently chosen from hydroxy, nitro, cyano, amino, halo, C₁-C₆ alkyl, C₁-C₆ haloalkyl, C₁-C₆ haloalkoxy, C₁-C₆ alkoxy, (C₁-C₆ alkoxy)C₁-C₆ alkoxy, mono-(C₁-C₆ alkyl)amino, di-(C₁-C₆ alkyl)amino, and amino(C₁-C₆ alkyl),
      heteroaryl, and
      substituted heteroaryl chosen from mono-, di-, and tri-substituted heteroaryl wherein the substitutents are independently chosen from hydroxy, nitro, cyano, amino, halo, C₁-C₆ alkyl, C₁-C₆ haloalkyl, C₁-C₆ haloalkoxy, C₁-C₆ alkoxy, (C₁-C₆ alkoxy)C₁-C₆ alkoxy, mono-(C₁-C₆ alkyl)amino, di-(C₁-C₆ alkyl)amino, and amino(C₁-C₆ alkyl);
R₁ is chosen from
   phenyl fused to a 5- to 7-membered heterocycloalkyl ring containing 1 or 2 heteroatoms chosen from N, O, and S, and
   substituted phenyl fused to a 5- to 7-membered heterocycloalkyl ring containing 1 or 2 heteroatoms chosen from N, O, and S, or heteroaryl, chosen from mono-, di-, and tri-substituted phenyl fused to a 5- to 7-membered heterocycloalkyl ring containing 1 or 2 heteroatoms chosen from N, O, and S, or heteroaryl, wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl;
Z₂ is chosen from
   phenylene,
   substituted phenylene chosen from mono-, di-, and tri-substituted phenylene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl),
   pyridylidene,
   substituted pyridylidene chosen from mono-, di-, and tri-substituted pyridylidene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl),
   naphthylidene, and
   substituted naphthylidene chosen from mono-, di-, and tri-substituted naphthylene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl);
Q is chosen from 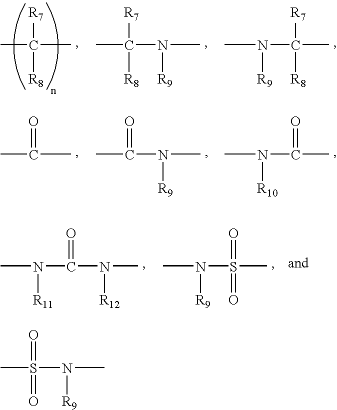
   wherein
      each occurrence of R₇ and R₈ is independently chosen from C₁-C₆ alkyl, sulfonamido, and halo; and n is chosen from 0, 1, 2, and 3; and
      R₉-R₁₂ are each independently chosen from
         hydrogen,
         C₁-C₆ alkyl,
         phenyl,
         substituted phenyl chosen from mono-, di-, and tri-substituted phenyl wherein the substituents are independently chosen from hydroxy, nitro, cyano, amino, halo, C₁-C₆ alkyl, C₁-C₆ alkoxy, (C₁-C₆ alkyloxy)C₁-C₆ alkoxy, C₁-C₆ perfluoroalkyl, C₁-C₆ perfluoroalkoxy, mono-(C₁-C₆ alkyl)amino, di(C₁-C₆ alkyl)amino, and amino(C₁-C₆ alkyl),
         heteroaryl, and
         substituted heteroaryl chosen from mono-, di-, and tri-substituted heteroaryl wherein the substituents are independently chosen from hydroxy, nitro, cyano, amino, halo, C₁-C₆ alkyl, C₁-C₆ alkoxy, (C₁-C₆ alkyloxy)C₁-C₆ alkoxy, C₁-C₆ perfluoroalkyl, C₁-C₆ perfluoroalkoxy, mono-(C₁-C₆ alkyl)amino, di(C₁-C₆ alkyl)amino, and amino(C₁-C₆ alkyl);
R₂ is chosen from
   C₁-C₇ alkyl,
   substituted C₁-C₇ alkyl chosen from mono-, di-, and tri-substituted C₁-C₇ alkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   C₃-C₇ cycloalkyl,
   substituted C₃-C₇ cycloalkyl chosen from mono-, di-, and tri-substituted C₃-C₇ cycloalkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   heterocycloalkyl,
   substituted heterocycloalkyl chosen from mono-, di-, and tri-substituted heterocycloalkyl, wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   C₁-C₆ alkoxy,
   substituted C₁-C₆ alkoxy chosen from mono-, di-, and tri-substituted C₁-C₆ alkoxy, wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   aryloxy,
   substituted aryloxy chosen from mono-, di-, and tri-substituted aryloxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   phenyl,
   substituted phenyl chosen from mono-, di-, and tri-substituted phenyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   heteroaryl, and
   substituted heteroaryl chosen from mono-, di-, and tri-substituted heteroaryl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl; and
R₃ is chosen from hydrogen, C₁-C₇ alkyl, C₃-C₇ cycloalkyl, (C₃-C₇ cycloalkyl)C₁-C₂ alkyl, heterocycloalkyl, and (heterocycloalkyl)C₁-C₂ alkyl.

Within certain embodiments, the invention provides at least one chemical entity chosen from compounds of Formula I: 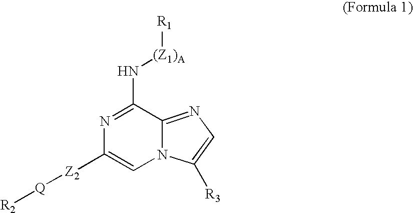
and pharmaceutically acceptable salts, solvates, crystal forms, diastereomers, and prodrugs thereof, wherein
A is chosen from 0 and 1;
Z₁ is chosen from 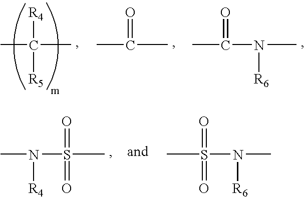
wherein
   each occurrence of R₄ and R₅ is independently chosen from hydrogen, C₁-C₆ alkyl, sulfonamido, and halo, and m is chosen from 0, 1, 2, and 3;
   R₆ is chosen from
      hydrogen,
      C₁-C₆ alkyl,
      phenyl,
      substituted phenyl chosen from mono-, di-, and tri-substituted phenyl wherein the substitutents are independently chosen from hydroxy, nitro, cyano, amino, halo, C₁-C₆ alkyl, C₁-C₆ haloalkyl, C₁-C₆ haloalkoxy, C₁-C₆ alkoxy, (C₁-C₆ alkoxy)C₁-C₆ alkoxy, mono-(C₁-C₆ alkyl)amino, di-(C₁-C₆ alkyl)amino, and amino(C₁-C₆ alkyl),
      heteroaryl, and
      substituted heteroaryl chosen from mono-, di-, and tri-substituted heteroaryl wherein the substitutents are independently chosen from hydroxy, nitro, cyano, amino, halo, C₁-C₆ alkyl, C₁-C₆ haloalkyl, C₁-C₆ haloalkoxy, C₁-C₆ alkoxy, (C₁-C₆ alkoxy)C₁-C₆ alkoxy, mono-(C₁-C₆ alkyl)amino, di-(C₁-C₆ alkyl)amino, and amino(C₁-C₆ alkyl);
R₁ is chosen from
   hydrogen,
   C₁-C₇ alkyl,
   C₁-C₆ alkoxy,
   C₃-C₇ cycloalkyl,
   (C₃-C₇ cycloalkyl)methyl,
   heterocycloalkyl,
   (heterocycloalkyl)C₁-C₂ alkyl,
   sulfonamido,
   (C₁-C₆ alkoxy)C₁-C₆ alkoxy,
   mono-(C₁-C₆ alkyl)amino,
   di-(C₁-C₆ alkyl)amino,
   mono-(C₁-C₆ alkyl)amino(C₁-C₆ alkyl),
   di(C₁-C₆ alkyl)amino(C₁-C₆ alkyl),
   phenyl,
   substituted phenyl chosen from mono-, di-, and tri-substituted phenyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
   phenyl fused to a 5- to 7-membered heterocycloalkyl ring containing 1 or 2 heteroatoms chosen from N, O, and S,
   substituted phenyl fused to a 5- to 7-membered heterocycloalkyl ring containing 1 or 2 heteroatoms chosen from N, O, and S, or heteroaryl, chosen from mono-, di-, and tri-substituted phenyl fused to a 5- to 7-membered heterocycloalkyl ring containing 1 or 2 heteroatoms chosen from N, O, and S, or heteroaryl, wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
   heteroaryl, and
   substituted heteroaryl chosen from mono-, di-, and tri-substituted heteroaryl, wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl;
Z₂ is chosen from
   phenylene,
   substituted phenylene chosen from mono-, di-, and tri-substituted phenylene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl),
   pyridylidene,
   substituted pyridylidene chosen from mono-, di-, and tri-substituted pyridylidene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl),
   naphthylidene, and
   substituted naphthylidene chosen from mono-, di-, and tri-substituted naphthylene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl);
Q is 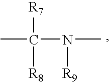
   wherein
      each occurrence of R₇ and R₈ is independently chosen from C₁-C₆ alkyl, sulfonamido, and halo; and n is chosen from 0, 1, 2, and 3; and
      R₉ is chosen from
         hydrogen,
         C₁-C₆ alkyl,
         phenyl,
         substituted phenyl chosen from mono-, di-, and tri-substituted phenyl wherein the substituents are independently chosen from hydroxy, nitro, cyano, amino, halo, C₁-C₆ alkyl, C₁-C₆ alkoxy, (C₁-C₆ alkyloxy)C₁-C₆ alkoxy, C₁-C₆ perfluoroalkyl, C₁-C₆ perfluoroalkoxy, mono-(C₁-C₆ alkyl)amino, di(C₁-C₆ alkyl)amino, and amino(C₁-C₆ alkyl),
         heteroaryl, and
         substituted heteroaryl chosen from mono-, di-, and tri-substituted heteroaryl wherein the substituents are independently chosen from hydroxy, nitro, cyano, amino, halo, C₁-C₆ alkyl, C₁-C₆ alkoxy, (C₁-C₆ alkyloxy)C₁-C₆ alkoxy, C₁-C₆ perfluoroalkyl, C₁-C₆ perfluoroalkoxy, mono-(C₁-C₆ alkyl)amino, di(C₁-C₆ alkyl)amino, and amino(C₁-C₆ alkyl);
R₂ is chosen from
   C₁-C₇ alkyl,
   substituted C₁-C₇ alkyl chosen from mono-, di-, and tri-substituted C₁-C₇ alkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   C₃-C₇ cycloalkyl,
   substituted C₃-C₇ cycloalkyl chosen from mono-, di-, and tri-substituted C₃-C₇ cycloalkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   heterocycloalkyl,
   substituted heterocycloalkyl chosen from mono-, di-, and tri-substituted heterocycloalkyl, wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   C₁-C₆ alkoxy,
   substituted C₁-C₆ alkoxy chosen from mono-, di-, and tri-substituted C₁-C₆ alkoxy, wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   aryloxy,
   substituted aryloxy chosen from mono-, di-, and tri-substituted aryloxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   phenyl,
   substituted phenyl chosen from mono-, di-, and tri-substituted phenyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   heteroaryl, and
   substituted heteroaryl chosen from mono-, di-, and tri-substituted heteroaryl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl; and
R₃ is chosen from hydrogen, C₁-C₇ alkyl, C₃-C₇ cycloalkyl, (C₃-C₇ cycloalkyl)C₁-C₂ alkyl, heterocycloalkyl, and (heterocycloalkyl)C₁-C₂ alkyl.

When referring to compounds of Formula I, in some embodiments, A is chosen from 0 and 1; and
Z₁ is chosen from 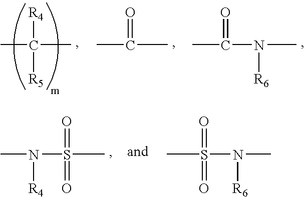
wherein
   each occurrence of R₄ and R₅ is independently chosen from hydrogen, C₁-C₆ alkyl, sulfonamido, and halo; and m is chosen from 0, 1, 2, and 3; and
   R₆ is chosen from
      hydrogen,
      C₁-C₆ alkyl,
      phenyl,
      substituted phenyl chosen from mono-, di-, and tri-substituted phenyl wherein the substitutents are independently chosen from hydroxy, nitro, cyano, amino, halo, C₁-C₆ alkyl, C₁-C₆ haloalkyl, C₁-C₆ haloalkoxy, C₁-C₆ alkoxy, (C₁-C₆ alkoxy)C₁-C₆ alkoxy, mono-(C₁-C₆ alkyl)amino, di-(C₁-C₆ alkyl)amino, and amino(C₁-C₆ alkyl), heteroaryl, and
      substituted heteroaryl chosen from mono-, di-, and tri-substituted heteroaryl wherein the substitutents are independently chosen from hydroxy, nitro, cyano, amino, halo, C₁-C₆ alkyl, C₁-C₆ haloalkyl, C₁-C₆ haloalkoxy, C₁-C₆ alkoxy, (C₁-C₆ alkoxy)C₁-C₆ alkoxy, mono-(C₁-C₆ alkyl)amino, di-(C₁-C₆ alkyl)amino, and amino(C₁-C₆ alkyl).
         In some embodiments, R₆ is chosen from hydrogen, C₁-C₆ alkyl, and phenyl. In some embodiments, A is 0 and Z₁ is absent. In some embodiments, A is 1 and Z₁ is 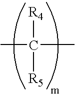
         wherein R₄ and R₅ are independently chosen from hydrogen, C₁-C₆ alkyl, sulfonamido, and halo; and m is chosen from 0, 1, 2, and 3. In some embodiments, R₄ and R₅ are hydrogen; and m is chosen from 0, 1, 2, and 3. In some embodiments, R₄ and R₅ are hydrogen; and m is chosen from 1, 2, and 3. In some embodiments, R₄ and R₅ are hydrogen; and m is chosen from 0 and 1. In some embodiments, R₄ and R₅ are hydrogen; and m is 1.

When referring to compounds of Formula I, in some embodiments, R₃ is chosen from hydrogen, C₁-C₇ alkyl, C₃-C₇ cycloalkyl, (C₃-C₇ cycloalkyl)C₁-C₂ alkyl, heterocycloalkyl, and (heterocycloalkyl)C₁-C₂ alkyl. In some embodiments, R₃ is hydrogen.

When referring to compounds of Formula I, in some embodiments, R₁ is chosen from
hydrogen,
C₁-C₇ alkyl,
C₁-C₆ alkoxy,
C₃-C₇ cycloalkyl,
(C₃-C₇ cycloalkyl)methyl,
heterocycloalkyl,
(heterocycloalkyl)C₁-C₂ alkyl,
sulfonamido,
(C₁-C₆ alkoxy)C₁-C₆ alkoxy,
mono-(C₁-C₆ alkyl)amino,
di-(C₁-C₆ alkyl)amino,
mono-(C₁-C₆ alkyl)amino(C₁-C₆ alkyl),
di(C₁-C₆ alkyl)amino(C₁-C₆ alkyl),
phenyl,
substituted phenyl chosen from mono-, di-, and tri-substituted phenyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
phenyl fused to a 5- to 7-membered heterocycloalkyl ring containing 1 or 2 heteroatoms chosen from N, O, and S,
substituted phenyl fused to a 5- to 7-membered heterocycloalkyl ring containing 1 or 2 heteroatoms chosen from N, O, and S, or heteroaryl, chosen from mono-, di-, and tri-substituted phenyl fused to a 5- to 7-membered heterocycloalkyl ring containing 1 or 2 heteroatoms chosen from N, O, and S, or heteroaryl, wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
heteroaryl, and
substituted heteroaryl chosen from mono-, di-, and tri-substituted heteroaryl, wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl.

When referring to compounds of Formula I, in some embodiments, R₁ is chosen from
C₃-C₇ cycloalkyl,
phenyl,
substituted phenyl chosen from mono-, di-, and tri-substituted phenyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆haloalkoxy, mono-(C₁-C₆ alkyl)amino, di-(C₁-C₆ alkyl)amino, di-(C₁-C₆ alkyl)amino(C₁-C₆ alkyl), mono-(C₁-C₆ alkyl)amino(C₁-C₆ alkyl), C₂-C₆ alkanoyl, and C₁-C₆ alkoxycarbonyl,
benzo[d]1,3-dioxolyl,
substituted benzo[d]1,3-dioxolyl chosen from mono-, di-, and tri-substituted benzo[d]1,3-dioxolyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆haloalkoxy, mono-(C₁-C₆ alkyl)amino, di-(C₁-C₆ alkyl)amino, di-(C₁-C₆ alkyl)amino(C₁-C₆ alkyl), mono-(C₁-C₆ alkyl)amino(C₁-C₆ alkyl), C₂-C₆ alkanoyl, and C₁-C₆ alkoxycarbonyl,
pyridyl, and
substituted pyridyl chosen from mono-, di-, and tri-substituted pyridyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆haloalkoxy, mono-(C₁-C₆ alkyl)amino, di-(C₁-C₆ alkyl)amino, di-(C₁-C₆ alkyl)amino(C₁-C₆ alkyl), mono-(C₁-C₆ alkyl)amino(C₁-C₆ alkyl), C₂-C₆ alkanoyl, and C₁-C₆ alkoxycarbonyl.

When referring to compounds of Formula I, in some embodiments, R₁ is chosen from
phenyl,
substituted phenyl chosen from mono-, di-, and tri-substituted phenyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
benzo[d]1,3-dioxolyl,
substituted benzo[d]1,3-dioxolyl chosen from mono-, di-, and tri-substituted benzo[d]1,3-dioxolyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
pyridyl,
substituted pyridyl chosen from mono-, di-, and tri-substituted pyridyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
pyrimidinyl, and
substituted pyrimidinyl chosen from mono-, di-, and tri-substituted pyrimidinyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl.

When referring to compounds of Formula I, in some embodiments, R₁ is chosen from
phenyl,
substituted phenyl chosen from mono-, di-, and tri-substituted phenyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆alkanoyl, and C₁-C₆ alkoxycarbonyl,
benzo[d]1,3-dioxolyl,
substituted benzo[d]1,3-dioxolyl chosen from mono-, di-, and tri-substituted benzo[d]1,3-dioxolyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆alkanoyl, and C₁-C₆ alkoxycarbonyl,
pyridyl, and
substituted pyridyl chosen from mono-, di-, and tri-substituted pyridyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆alkanoyl, and C₁-C₆ alkoxycarbonyl.

When referring to compounds of Formula I, in some embodiments, Q is chosen from 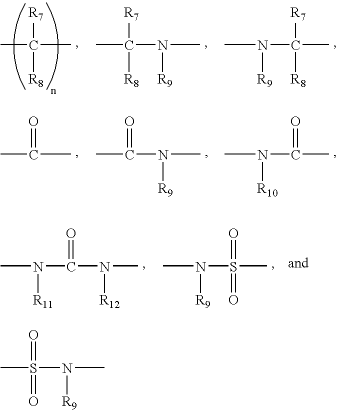
wherein
   each occurrence of R₇ and R₈ is independently chosen from C₁-C₆ alkyl, sulfonamido, and halo;
   n is chosen from 0, 1, 2, and 3; and
   R₉-R₁₂ are each independently chosen from
      hydrogen,
      C₁-C₆ alkyl,
      phenyl,
      substituted phenyl chosen from mono-, di-, and tri-substituted phenyl wherein the substituents are independently chosen from hydroxy, nitro, cyano, amino, halo, C₁-C₆ alkyl, C₁-C₆ alkoxy, (C₁-C₆ alkyloxy)C₁-C₆ alkoxy, C₁-C₆ perfluoroalkyl, C₁-C₆ perfluoroalkoxy, mono-(C₁-C₆ alkyl)amino, di(C₁-C₆ alkyl)amino, and amino(C₁-C₆ alkyl),
      heteroaryl, and
      substituted heteroaryl chosen from mono-, di-, and tri-substituted heteroaryl wherein the substituents are independently chosen from hydroxy, nitro, cyano, amino, halo, C₁-C₆ alkyl, C₁-C₆ alkoxy, (C₁-C₆ alkyloxy)C₁-C₆ alkoxy, C₁-C₆ perfluoroalkyl, C₁-C₆ perfluoroalkoxy, mono-(C₁-C₆ alkyl)amino, di(C₁-C₆ alkyl)amino, and amino(C₁-C₆ alkyl).
         When referring to compounds of Formula I, in some embodiments, each occurrence of R₇ and R₈ is independently chosen from C₁-C₆ alkyl, sulfonamido, and halo; and n is chosen from 1 and 2. In some embodiments, R₉-R₁₂ are each independently chosen from hydrogen, C₁-C₆ alkyl, and phenyl.

When referring to compounds of Formula I, in some embodiments, Z₂ is chosen from
phenylene,
substituted phenylene chosen from mono-, di-, and tri-substituted phenylene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl),
pyridylidene,
substituted pyridylidene chosen from mono-, di-, and tri-substituted pyridylidene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl),
naphthylidene, and
substituted naphthylidene chosen from mono-, di-, and tri-substituted naphthylene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

When referring to compounds of Formula I, in some embodiments, Z₂ is chosen from
substituted phenylene chosen from mono-, di-, and tri-substituted phenylene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl),
pyridylidene,
substituted pyridylidene chosen from mono-, di-, and tri-substituted pyridylidene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl), and
substituted naphthylidene chosen from mono-, di-, and tri-substituted naphthylene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

When referring to compounds of Formula I, in some embodiments, Z₂ is chosen from
phenylene,
substituted phenylene chosen from mono-, di-, and tri-substituted phenylene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl),
pyridylidene, and
substituted pyridylidene chosen from mono-, di-, and tri-substituted pyridylidene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

When referring to compounds of Formula I, in some embodiments, Z₂ is
substituted phenylene chosen from mono-, di-, and tri-substituted phenylene wherein the substituents are independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

When referring to compounds of Formula I, in some embodiments, R₂ is chosen from
C₁-C₇ alkyl,
substituted C₁-C₇ alkyl chosen from mono-, di-, and tri-substituted C₁-C₇ alkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
C₃-C₇ cycloalkyl,
substituted C₃-C₇ cycloalkyl chosen from mono-, di-, and tri-substituted C₃-C₇ cycloalkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
heterocycloalkyl,
substituted heterocycloalkyl chosen from mono-, di-, and tri-substituted heterocycloalkyl, wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
C₁-C₆ alkoxy,
substituted C₁-C₆ alkoxy chosen from mono-, di-, and tri-substituted C₁-C₆ alkoxy, wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
aryloxy,
substituted aryloxy chosen from mono-, di-, and tri-substituted aryloxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
phenyl,
substituted phenyl chosen from mono-, di-, and tri-substituted phenyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
heteroaryl, and
substituted heteroaryl chosen from mono-, di-, and tri-substituted heteroaryl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl.

When referring to compounds of Formula I, in some embodiments, R₂ is chosen from
(C₃-C₇ cycloalkyl)methyl,
substituted (C₃-C₇ cycloalkyl)methyl chosen from mono-, di-, and tri-substituted (C₃-C₇ cycloalkyl)methyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
(heterocycloalkyl)C₁-C₂ alkyl,
substituted (heterocycloalkyl)C₁-C₂ alkyl chosen from mono-, di-, and tri-substituted (heterocycloalkyl)C₁-C₂ alkyl, wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
(C₁-C₆ alkoxy)C₁-C₆ alkoxy, and
substituted (C₁-C₆ alkoxy)C₁-C₆ alkoxy chosen from mono-, di-, and tri-substituted (C₁-C₆ alkoxy)C₁-C₆ alkoxy, wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl.

When referring to compounds of Formula I, in some embodiments, R₂ is chosen from
C₃-C₇ cycloalkyl,
substituted C₃-C₇ cycloalkyl chosen from mono-, di-, and tri-substituted C₃-C₇ cycloalkyl wherein the substituents are independently chosen from hydroxy, cyano, amino, halo, C₁-C₄ alkyl, C₁-C₄ alkoxy, C₁-C₂ haloalkyl, C₁-C₂ haloalkoxy, mono-(C₁-C₄ alkyl)amino, and di-(C₁-C₄ alkyl)amino,
(C₃-C₇ cycloalkyl)methyl,
substituted (C₃-C₇ cycloalkyl)methyl chosen from mono-, di-, and tri-substituted (C₃-C₇ cycloalkyl)methyl wherein the substituents are independently chosen from hydroxy, cyano, amino, halo, C₁-C₄ alkyl, C₁-C₄ alkoxy, C₁-C₂ haloalkyl, C₁-C₂ haloalkoxy, mono-(C₁-C₄ alkyl)amino, and di-(C₁-C₄ alkyl)amino,
heterocycloalkyl,
substituted heterocycloalkyl chosen from mono-, di-, and tri-substituted heterocycloalkyl, wherein the substituents are independently chosen from hydroxy, cyano, amino, halo, C₁-C₄ alkyl, C₁-C₄ alkoxy, C₁-C₂ haloalkyl, C₁-C₂ haloalkoxy, mono-(C₁-C₄ alkyl)amino, and di-(C₁-C₄ alkyl)amino,
(heterocycloalkyl)C₁-C₂ alkyl,
substituted (heterocycloalkyl)C₁-C₂ alkyl chosen from mono-, di-, and tri-substituted (heterocycloalkyl)C₁-C₂ alkyl, wherein the substituents are independently chosen from hydroxy, cyano, amino, halo, C₁-C₄ alkyl, C₁-C₄ alkoxy, C₁-C₂ haloalkyl, C₁-C₂ haloalkoxy, mono-(C₁-C₄ alkyl)amino, and di-(C₁-C₄ alkyl)amino,
C₁-C₆ alkoxy,
substituted C₁-C₆ alkoxy chosen from mono-, di-, and tri-substituted C₁-C₆ alkoxy, wherein the substituents are independently chosen from hydroxy, cyano, amino, halo, C₁-C₄ alkyl, C₁-C₄ alkoxy, C₁-C₂ haloalkyl, C₁-C₂ haloalkoxy, mono-(C₁-C₄ alkyl)amino, and di-(C₁-C₄ alkyl)amino,
(C₁-C₆ alkoxy)C₁-C₆ alkoxy,
substituted (C₁-C₆ alkoxy)C₁-C₆ alkoxy chosen from mono-, di-, and tri-substituted (C₁-C₆ alkoxy)C₁-C₆ alkoxy, wherein the substituents are independently chosen from hydroxy, cyano, amino, halo, C₁-C₄ alkyl, C₁-C₄ alkoxy, C₁-C₂ haloalkyl, C₁-C₂ haloalkoxy, mono-(C₁-C₄ alkyl)amino, and di-(C₁-C₄ alkyl)amino,
aryloxy,
substituted aryloxy chosen from mono-, di-, and tri-substituted aryloxy wherein the substituents are independently chosen from hydroxy, cyano, amino, halo, C₁-C₄ alkyl, C₁-C₄ alkoxy, C₁-C₂ haloalkyl, C₁-C₂ haloalkoxy, mono-(C₁-C₄ alkyl)amino, and di-(C₁-C₄ alkyl)amino,
phenyl,
substituted phenyl chosen from mono-, di-, and tri-substituted phenyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
pyridyl,
substituted pyridyl chosen from mono-, di-, and tri-substituted pyridyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
pyrimidinyl, and
substituted pyrimidinyl chosen from mono-, di-, and tri-substituted pyrimidinyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl.

When referring to compounds of Formula I, in some embodiments, R₂ is chosen from
phenyl,
substituted phenyl chosen from mono-, di-, and tri-substituted phenyl wherein the substituents are indepedently chosen from hydroxy, nitro, cyano, amino, halo, C₁-C₆ alkyl, C₁-C₆ alkoxy, C₁-C₂ haloalkyl, C₁-C₂ haloalkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, piperidinyl, piperazinyl, and morpholinyl,
pyridyl, and
substituted pyridyl chosen from mono-, di-, and tri-substituted pyridyl wherein the substituents are indepedently chosen from hydroxy, nitro, cyano, amino, halo, C₁-C₆ alkyl, C₁-C₆ alkoxy, C₁-C₂ haloalkyl, C₁-C₂ haloalkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, piperidinyl, piperazinyl, and morpholinyl.

When referring to compounds of Formula I, in some embodiments, R₂ is chosen from
phenyl substituted with at least one branched C₃-C₆alkyl, and further substituted with 0 to 2 substituents independently chosen from hydroxy, nitro, cyano, amino, halo, C₁-C₆ alkyl, C₁-C₆ alkoxy, C₁-C₂ haloalkyl, C₁-C₂ haloalkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, piperidinyl, piperazinyl, and morpholinyl, and
pyridyl substituted with at least one branched C₃-C₆alkyl, and further substituted with 0 to 2 substituents independently chosen from hydroxy, nitro, cyano, amino, halo, C₁-C₆ alkyl, C₁-C₆ alkoxy, C₁-C₂ haloalkyl, C₁-C₂ haloalkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, piperidinyl, piperazinyl, and morpholinyl.

When referring to compounds of Formula I, in some embodiments, R₂-Q- is chosen from
halo-C₁-C₄ alkyl,
phenoxy and
substituted phenoxy chosen from mono-, di-, and tri-substituted phenoxy wherein the substituents are independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

In some embodiments,
R₂-Q- is chosen from
   chloro-C₁-C₄ alkyl,
   phenoxy, and
   substituted phenoxy chosen from mono-, di-, and tri-substituted phenoxy wherein the substituents are independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄alkyl)amino, di-(C₁-C₄alkyl)amino, and amino(C₁-C₄alkyl).

In some embodiments, R₂-Q- is halo-C₁-C₄ alkyl. In some embodiments, R₂-Q- is chosen from
phenoxy and
substituted phenoxy chosen from mono-, di-, and tri-substituted phenoxy wherein the substituents are independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

When referring to compounds of Formula I, in some embodiments,
R₆ is chosen from hydrogen, C₁-C₆ alkyl, and phenyl;
R₁ is chosen from
   phenyl,
   substituted phenyl chosen from mono-, di-, and tri-substituted phenyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
   benzo[d]1,3-dioxolyl,
   substituted benzo[d]1,3-dioxolyl chosen from mono-, di-, and tri-substituted benzo[d]1,3-dioxolyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
   pyridyl,
   substituted pyridyl chosen from mono-, di-, and tri-substituted pyridyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
   pyrimidinyl, and
   substituted pyrimidinyl chosen from mono-, di-, and tri-substituted pyrimidinyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl;
Z₂ is chosen from
   phenylene,
   substituted phenylene chosen from mono-, di-, and tri-substituted phenylene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl),
   pyridylidene,
   substituted pyridylidene chosen from mono-, di-, and tri-substituted pyridylidene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl),
   naphthylidene, and
   substituted naphthylidene chosen from mono-, di-, and tri-substituted naphthylene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl);
R₂-Q- is chosen from
   chloro-C₁-C₄ alkyl,
   phenoxy, and
   substituted phenoxy chosen from mono-, di-, and tri-substituted phenoxy wherein the substituents are independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄alkyl)amino, di-(C₁-C₄alkyl)amino, and amino(C₁-C₄alkyl).

When referring to compounds of Formula I, in some embodiments, R₆ is chosen from hydrogen, C₁-C₆ alkyl, and phenyl;
R₁ is chosen from
   phenyl,
   substituted phenyl chosen from mono-, di-, and tri-substituted phenyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
   benzo[d]1,3-dioxolyl,
   substituted benzo[d]1,3-dioxolyl chosen from mono-, di-, and tri-substituted benzo[d]1,3-dioxolyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
   pyridyl,
   substituted pyridyl chosen from mono-, di-, and tri-substituted pyridyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
   pyrimidinyl, and
   substituted pyrimidinyl chosen from mono-, di-, and tri-substituted pyrimidinyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl;
Z₂ is chosen from
   phenylene,
   substituted phenylene chosen from mono-, di-, and tri-substituted phenylene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl),
   pyridylidene,
   substituted pyridylidene chosen from mono-, di-, and tri-substituted pyridylidene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl),
   naphthylidene, and
   substituted naphthylidene chosen from mono-, di-, and tri-substituted naphthylene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl);
Q is chosen from 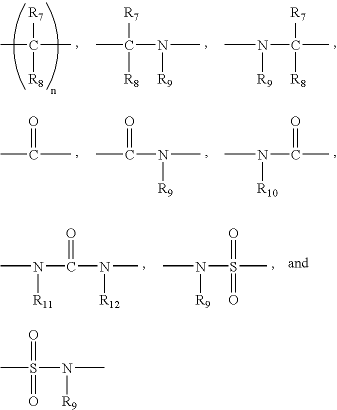
   wherein
      each occurrence of R₇ and R₈ is independently chosen from C₁-C₆ alkyl, sulfonamido, and halo, and n is chosen from 0, 1, 2, and 3; and
      R₉-R₁₂ are each independently chosen from hydrogen, C₁-C₆ alkyl, and phenyl;
   R₂ is chosen from
      C₃-C₇ cycloalkyl,
      substituted C₃-C₇ cycloalkyl chosen from mono-, di-, and tri-substituted C₃-C₇ cycloalkyl wherein the substituents are independently chosen from hydroxy, cyano, amino, halo, C₁-C₄ alkyl, C₁-C₄ alkoxy, C₁-C₂ haloalkyl, C₁-C₂ haloalkoxy, mono-(C₁-C₄ alkyl)amino, and di-(C₁-C₄ alkyl)amino,
      (C₃-C₇ cycloalkyl)methyl,
      substituted (C₃-C₇ cycloalkyl)methyl chosen from mono-, di-, and tri-substituted (C₃-C₇ cycloalkyl)methyl wherein the substituents are independently chosen from hydroxy, cyano, amino, halo, C₁-C₄ alkyl, C₁-C₄ alkoxy, C₁-C₂ haloalkyl, C₁-C₂ haloalkoxy, mono-(C₁-C₄ alkyl)amino, and di-(C₁-C₄ alkyl)amino,
      heterocycloalkyl,
      substituted heterocycloalkyl chosen from mono-, di-, and tri-substituted heterocycloalkyl, wherein the substituents are independently chosen from hydroxy, cyano, amino, halo, C₁-C₄ alkyl, C₁-C₄ alkoxy, C₁-C₂ haloalkyl, C₁-C₂ haloalkoxy, mono-(C₁-C₄ alkyl)amino, and di-(C₁-C₄ alkyl)amino,
      (heterocycloalkyl)C₁-C₂ alkyl,
      substituted (heterocycloalkyl)C₁-C₂ alkyl chosen from mono-, di-, and tri-substituted (heterocycloalkyl)C₁-C₂ alkyl, wherein the substituents are independently chosen from hydroxy, cyano, amino, halo, C₁-C₄ alkyl, C₁-C₄ alkoxy, C₁-C₂ haloalkyl, C₁-C₂ haloalkoxy, mono-(C₁-C₄ alkyl)amino, and di-(C₁-C₄ alkyl)amino,
      C₁-C₆ alkoxy,
      substituted C₁-C₆ alkoxy chosen from mono-, di-, and tri-substituted C₁-C₆ alkoxy, wherein the substituents are independently chosen from hydroxy, cyano, amino, halo, C₁-C₄ alkyl, C₁-C₄ alkoxy, C₁-C₂ haloalkyl, C₁-C₂ haloalkoxy, mono-(C₁-C₄ alkyl)amino, and di-(C₁-C₄ alkyl)amino,
      (C₁-C₆ alkoxy)C₁-C₆ alkoxy,
      substituted (C₁-C₆ alkoxy)C₁-C₆ alkoxy chosen from mono-, di-, and tri-substituted (C₁-C₆ alkoxy)C₁-C₆ alkoxy, wherein the substituents are independently chosen from hydroxy, cyano, amino, halo, C₁-C₄ alkyl, C₁-C₄ alkoxy, C₁-C₂ haloalkyl, C₁-C₂ haloalkoxy, mono-(C₁-C₄ alkyl)amino, and di-(C₁-C₄ alkyl)amino,
      aryloxy,
      substituted aryloxy chosen from mono-, di-, and tri-substituted aryloxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
      phenyl,
      substituted phenyl chosen from mono-, di-, and tri-substituted phenyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
      pyridyl,
      substituted pyridyl chosen from mono-, di-, and tri-substituted pyridyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂C₆ alkanoyl, C₁-C₆ alkoxycarbonyl, and heterocycloalkyl,
      pyrimidinyl, and
      substituted pyrimidinyl chosen from mono-, di-, and tri-substituted pyrimidinyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl.

In another embodiment, the invention provides at least one chemical entity chosen from compounds of Formula 2: 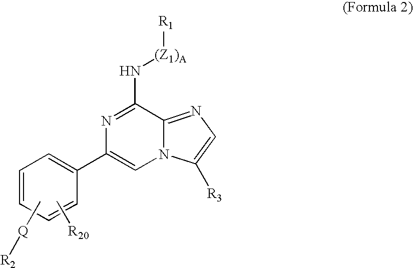
and pharmaceutically acceptable salts, solvates, crystal forms, diastereomers, and prodrugs thereof, wherein
R₁, Q, R₂, R₃, Z₁, and A are as described for compounds of Formula I, and further wherein
R₂₀ represents 0, 1, 2, or 3 substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl). In some embodiments, R₂₀ represents 1, 2, or 3 substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl). In some embodiments, R₂₀ represents one substituent chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

When referring to compounds of Formula 2, in some embodiments, R₁ is chosen from
phenyl,
substituted phenyl chosen from mono-, di-, and tri-substituted phenyl wherein the substituents are chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
benzo[d]1,3-dioxolyl,
substituted benzo[d]1,3-dioxolyl chosen from mono-, di-, and tri-substituted benzo[d]1,3-dioxolyl wherein the substituents are chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
pyridyl, and
substituted benzo[d]1,3-dioxolyl chosen from mono-, di-, and tri-substituted benzo[d]1,3-dioxolyl wherein the substituents are chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl.

Some embodiments provide at least one chemical entity chosen from compounds of Formula 3 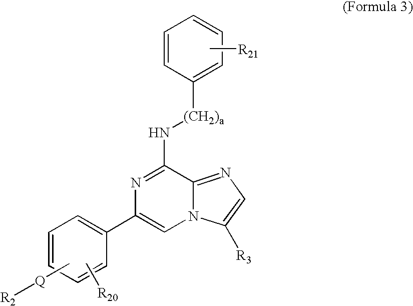
and pharmaceutically acceptable salts, solvates, crystal forms, diastereomers, and prodrugs thereof, wherein Q, R₂ and R₃ are as described for compounds of Formula 1; a is chosen from 0, 1, 2 and 3; R₂₀ represents 0 to 3 substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl); and R₂₁ represents 0 to 3 substituents independently chosen from hydroxy, nitro, cyano, carboxy, amino, sulfonamido, halo, C₁-C₆ alkyl, C₁-C₂ haloalkyl, C₁-C₂ haloalkoxy, C₁-C₆ alkoxy, mono-(C₁-C₆ alkyl)amino, di-(C₁-C₆ alkyl)amino, mono-(C₁-C₆ alkyl)amino(C₁-C₆ alkyl), di(C₁-C₆ alkyl)amino(C₁-C₆ alkyl), C₂-C₆alkanoyl, and C₁-C₆alkoxycarbonyl. In some embodiments, R₂₀ represents 1, 2, or 3 substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl). In some embodiments, R₂₀ represents 1 substituent chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

Some embodiments provide at least one chemical entity chosen from compounds of Formula 4 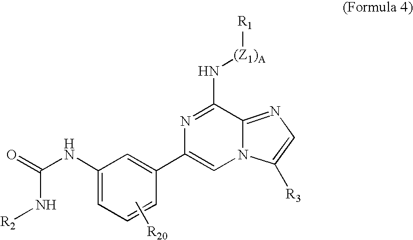
and pharmaceutically acceptable salts, solvates, crystal forms, diastereomers, and prodrugs thereof, wherein R₁, R₂, R₃, Z₁, and A are as described for compounds of Formula 1, and further wherein R₂₀ represents 0 to 3 substituents independently chosen from hydroxy, nitro, cyano, amino, halo, C₁-C₆ alkyl, C₁-C₂haloalkyl, C₁-C₂haloalkoxy, C₁-C₆alkoxy, mono-(C₁-C₄alkyl)amino, di-(C₁-C₄alkyl)amino, and amino(C₁-C₄ alkyl). In some embodiments, R₂₀ represents 1, 2, or 3 substituents independently chosen from hydroxy, nitro, cyano, amino, halo, C₁-C₆ alkyl, C₁-C₂haloalkyl, C₁-C₂haloalkoxy, C₁-C₆alkoxy, mono-(C₁-C₄alkyl)amino, di-(C₁-C₄alkyl)amino, and amino(C₁-C₄ alkyl). In some embodiments, R₂₀ represents one substituent chosen from hydroxy, nitro, cyano, amino, halo, C₁-C₆ alkyl, C₁-C₂haloalkyl, C₁-C₂haloalkoxy, C₁-C₆alkoxy, mono-(C₁-C₄alkyl)amino, di-(C₁-C₄alkyl)amino, and amino(C₁-C₄ alkyl).

Some embodiments provide at least one chemical entity chosen from compounds of Formula 5 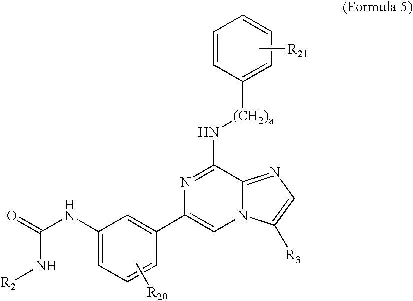
and pharmaceutically acceptable salts, solvates, crystal forms, diastereomers, and prodrugs thereof, wherein R₃ is as described for compounds of Formula 1, and further wherein
a is chosen from 0, 1, 2 and 3;
R₂₀ represents 0 to 3 substituents independently chosen from hydroxy, nitro, cyano, amino, halo, C₁-C₆ alkyl, C₁-C₂haloalkyl, C₁-C₂haloalkoxy, C₁-C₆alkoxy, mono-(C₁-C₄alkyl)amino, di-(C₁-C₄alkyl)amino, and amino(C₁-C₄ alkyl);
R₂₁ represents 0 to 3 substituents independently chosen from hydroxy, nitro, cyano, carboxy, amino, sulfonamido, halo, C₁-C₆ alkyl, C₁-C₂ haloalkyl, C₁-C₂ haloalkoxy, C₁-C₆ alkoxy, mono-(C₁-C₆ alkyl)amino, di-(C₁-C₆ alkyl)amino, mono-(C₁-C₆ alkyl)amino(C₁-C₆ alkyl), di(C₁-C₆ alkyl)amino(C₁-C₆ alkyl), C₂-C₆alkanoyl, and C₁-C₆alkoxycarbonyl; and
R₂ is chosen from
   phenyl,
   substituted phenyl chosen from mono-, di-, and tri-substituted phenyl wherein the substituents are indepedently chosen from hydroxy, nitro, cyano, amino, halo, C₁-C₆ alkyl, C₁-C₆ alkoxy, C₁-C₂ haloalkyl, C₁-C₂ haloalkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, piperidinyl, piperazinyl, and morpholinyl,
   pyridyl, and
   substituted pyridyl chosen from mono-, di-, and tri-substituted pyridyl wherein the substituents are indepedently chosen from hydroxy, nitro, cyano, amino, halo, C₁-C₆ alkyl, C₁-C₆ alkoxy, C₁-C₂ haloalkyl, C₁-C₂ haloalkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, piperidinyl, piperazinyl, and morpholinyl.
      In some embodiments, R₂₀ represents 1, 2, or 3 substituents independently chosen from hydroxy, nitro, cyano, amino, halo, C₁-C₆ alkyl, C₁-C₂haloalkyl, C₁-C₂haloalkoxy, C₁-C₆alkoxy, mono-(C₁-C₄alkyl)amino, di-(C₁-C₄alkyl)amino, and amino(C₁-C₄ alkyl). In some embodiments, R₂₀ represents 1 substituent chosen from hydroxy, nitro, cyano, amino, halo, C₁-C₆ alkyl, C₁-C₂haloalkyl, C₁-C₂haloalkoxy, C₁-C₆alkoxy, mono-(C₁-C₄alkyl)amino, di-(C₁-C₄alkyl)amino, and amino(C₁-C₄ alkyl).

Some embodiments provide at least one chemical entity chosen from compounds of Formula 6 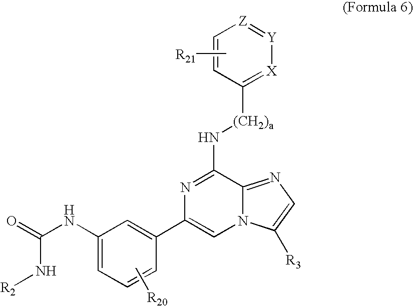
and pharmaceutically acceptable salts, solvates, crystal forms, diastereomers, and prodrugs thereof, wherein R₃ is as described for compounds of Formula 1, and R₂ is chosen from
   C₁-C₆ alkyl,
   substituted C₁-C₆ alkyl chosen from mono-, di-, and tri-substituted C₁-C₆ alkyl wherein the substituents are chosen from hydroxy, nitro, cyano, amino; halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   C₃-C₇ cycloalkyl,
   substituted C₃-C₇ cycloalkyl alkyl chosen from mono-, di-, and tri-substituted C₃-C₇ cycloalkyl wherein the substituents are chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   (C₃-C₇ cycloalkyl)methyl,
   substituted (C₃-C₇ cycloalkyl)methyl chosen from mono-, di-, and tri-substituted (C₃-C₇ cycloalkyl)methyl wherein the substituents are chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   heterocycloalkyl,
   substituted heterocycloalkyl chosen from mono-, di-, and tri-substituted heterocycloalkyl wherein the substituents are chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   (heterocycloalkyl)C₁-C₂ alkyl,
   substituted (heterocycloalkyl)C₁-C₂ alkyl, chosen from mono-, di-, and tri-substituted (heterocycloalkyl)C₁-C₂ alkyl, wherein the substituents are chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   C₁-C₆ alkoxy,
   substituted C₁-C₆ alkoxy chosen from mono-, di-, and tri-substituted C₁-C₆ alkoxy wherein the substituents are chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   aryloxy,
   substituted aryloxy chosen from mono-, di-, and tri-substituted aryloxy wherein the substituents are chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   phenyl,
   substituted phenyl chosen from mono-, di-, and tri-substituted phenyl wherein the substituents are chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   heteroaryl, and
   substituted heteroaryl chosen from mono-, di-, and tri-substituted heteroaryl wherein the substituents are chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl;
a is chosen from 0, 1, 2 and 3;
X, Y, and Z are chosen from nitrogen and CH, wherein one and only one of X, Y, and Z is nitrogen; and
R₂₁ represents 0 to 4 substituents independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl.
In some embodiments, R₂ is chosen from
   phenyl,
   substituted phenyl chosen from mono-, di-, and tri-substituted phenyl wherein the substituents are independently chosen from hydroxy, nitro, cyano, amino, halo, C₁-C₆ alkyl, C₁-C₆ alkoxy, C₁-C₂ haloalkyl, C₁-C₂ haloalkoxy, mono-(C₁-C₆ alkyl)amino, di-(C₁-C₆ alkyl)amino, amino(C₁-C₆ alkyl), C₁-C₆ alkylthio, di-(C₁-C₆ alkyl)amino(C₁-C₆ alkyl), mono-(C₁-C₆ alkyl)amino(C₁-C₆ alkyl), C₃-C₇ cycloalkyl, piperidinyl, piperazinyl, and morpholinyl,
   pyridyl, and
   substituted pyridyl chosen from mono-, di-, and tri-substituted pyridyl wherein the substituents are independently chosen from hydroxy, nitro, cyano, amino, halo, C₁-C₆ alkyl, C₁-C₆ alkoxy, C₁-C₂ haloalkyl, C₁-C₂ haloalkoxy, mono-(C₁-C₆ alkyl)amino, di-(C₁-C₆ alkyl)amino, amino(C₁-C₆ alkyl), C₁-C₆ alkylthio, di-(C₁-C₆ alkyl)amino(C₁-C₆ alkyl), mono-(C₁-C₆ alkyl)amino(C₁-C₆ alkyl), C₃-C₇ cycloalkyl, piperidinyl, piperazinyl, and morpholinyl.

Some embodiments provide at least one chemical entity chosen from compounds of Formula 7 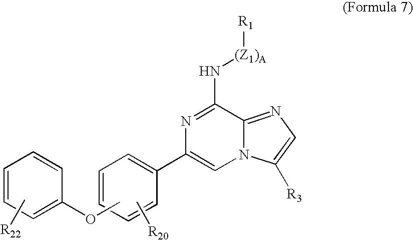
and pharmaceutically acceptable salts, solvates, crystal forms, diastereomers, and prodrugs thereof wherein Z₁, R₃, and A are as described for Formula 1, and further wherein R₁ is chosen from
   C₃-C₇ cycloalkyl,
   phenyl,
   substituted phenyl chosen from mono-, di-, and tri-substituted phenyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆haloalkoxy, mono-(C₁-C₆ alkyl)amino, di-(C₁-C₆ alkyl)amino, di-(C₁-C₆ alkyl)amino(C₁-C₆ alkyl), mono-(C₁-C₆ alkyl)amino(C₁-C₆ alkyl), C₂-C₆ alkanoyl, and C₁-C₆ alkoxycarbonyl,
   benzo[d]1,3-dioxolyl,
   substituted benzo[d]1,3-dioxolyl chosen from mono-, di-, and tri-substituted benzo[d]1,3-dioxolyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆haloalkoxy, mono-(C₁-C₆ alkyl)amino, di-(C₁-C₆ alkyl)amino, di-(C₁-C₆ alkyl)amino(C₁-C₆ alkyl), mono-(C₁-C₆ alkyl)amino(C₁-C₆ alkyl), C₂-C₆ alkanoyl, and C₁-C₆ alkoxycarbonyl,
   pyridyl, and
   substituted pyridyl chosen from mono-, di-, and tri-substituted pyridyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆haloalkoxy, mono-(C₁-C₆ alkyl)amino, di-(C₁-C₆ alkyl)amino, di-(C₁-C₆ alkyl)amino(C₁-C₆ alkyl), mono-(C₁-C₆ alkyl)amino(C₁-C₆ alkyl), C₂-C₆ alkanoyl, and C₁-C₆ alkoxycarbonyl;
R₂₀ represents 0 to 3 substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl); and
R₂₂ represents 0 to 4 substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).
In some embodiments, R₂₀ represents 1, 2, or 3 substituents independently chosen from hydroxy, nitro, cyano, amino, halo, C₁-C₆ alkyl, C₁-C₂haloalkyl, C₁-C₂haloalkoxy, C₁-C₆alkoxy, mono-(C₁-C₄alkyl)amino, di-(C₁-C₄alkyl)amino, and amino(C₁-C₄ alkyl). In some embodiments, R₂₀ represents 1 substituent chosen from hydroxy, nitro, cyano, amino, halo, C₁-C₆ alkyl, C₁-C₂haloalkyl, C₁-C₂haloalkoxy, C₁-C₆alkoxy, mono-(C₁-C₄alkyl)amino, di-(C₁-C₄alkyl)amino, and amino(C₁-C₄ alkyl).

Some embodiments provide at least one chemical entity chosen from compounds of Formula 8 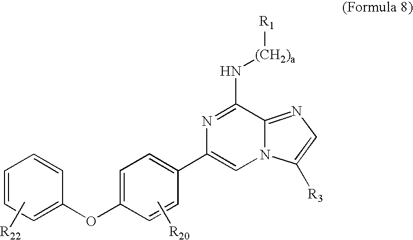
and pharmaceutically acceptable salts, solvates, crystal forms, diastereomers, and prodrugs thereof, wherein R₁, R₃, R₂₀, and R₂₂ are as described for compounds of Formula 7; and further wherein a is chosen from 0 and 1.

Some embodiments provide at least one chemical entity chosen from compounds of Formula 9 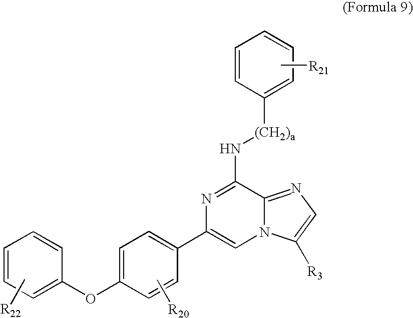
and pharmaceutically acceptable salts, solvates, crystal forms, diastereomers, and prodrugs thereof, wherein R₃, R₂₀, R₂₂, and a are as described for compounds of Formula 8; and further wherein R₂₁ represents 0 to 3 substituents independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl.

Some embodiments provide at least one chemical entity chosen from compounds of Formula 10 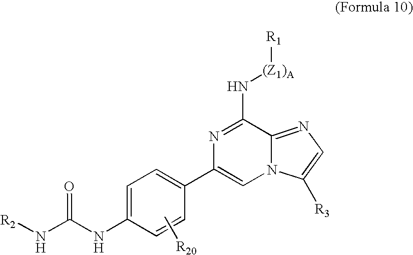
and pharmaceutically acceptable salts, solvates, crystal forms, diastereomers, and prodrugs thereof, wherein R₁, R₃, Z₁, and A are as described for Formula 1; and R₂ is chosen from
   C₁-C₆ alkyl,
   substituted C₁-C₆ alkyl chosen from mono-, di-, and tri-substituted C₁-C₆ alkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, di-(C₁-C₆ alkyl)amino(C₁-C₆ alkyl), mono-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   C₃-C₇ cycloalkyl,
   substituted C₃-C₇ cycloalkyl alkyl chosen from mono-, di-, and tri-substituted C₃-C₇ cycloalkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, di-(C₁-C₆ alkyl)amino(C₁-C₆ alkyl), mono-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   (C₃-C₇ cycloalkyl)methyl,
   substituted (C₃-C₇ cycloalkyl)methyl chosen from mono-, di-, and tri-substituted (C₃-C₇ cycloalkyl)methyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, di-(C₁-C₆ alkyl)amino(C₁-C₆ alkyl), mono-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   heterocycloalkyl,
   substituted heterocycloalkyl chosen from mono-, di-, and tri-substituted heterocycloalkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, di-(C₁-C₆ alkyl)amino(C₁-C₆ alkyl), mono-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   (heterocycloalkyl)C₁-C₂ alkyl,
   substituted (heterocycloalkyl)C₁-C₂ alkyl chosen from mono-, di-, and tri-substituted (heterocycloalkyl)C₁-C₂ alkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, di-(C₁-C₆ alkyl)amino(C₁-C₆ alkyl), mono-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   C₁-C₆ alkoxy,
   substituted C₁-C₆ alkoxy chosen from mono-, di-, and tri-substituted C₁-C₆ alkoxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, di-(C₁-C₆ alkyl)amino(C₁-C₆ alkyl), mono-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   (C₁-C₆ alkoxy)C₁-C₆ alkoxy,
   substituted (C₁-C₆ alkoxy)C₁-C₆ alkoxy chosen from mono-, di-, and tri-substituted (C₁-C₆ alkoxy)C₁-C₆ alkoxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, di-(C₁-C₆ alkyl)amino(C₁-C₆ alkyl), mono-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   aryloxy,
   substituted aryloxy chosen from mono-, di-, and tri-substituted aryloxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, di-(C₁-C₆ alkyl)amino(C₁-C₆ alkyl), mono-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   phenyl,
   substituted phenyl chosen from mono-, di-, and tri-substituted phenyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, di-(C₁-C₆ alkyl)amino(C₁-C₆ alkyl), mono-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   heteroaryl, and
   substituted heteroaryl chosen from mono-, di-, and tri-substituted heteroaryl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, di-(C₁-C₆ alkyl)amino(C₁-C₆ alkyl), mono-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl; and
R₂₀ represents 0 to 3 substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).
In some embodiments, R₂₀ represents 1, 2, or 3 substituents independently chosen from hydroxy, nitro, cyano, amino, halo, C₁-C₆ alkyl, C₁-C₂haloalkyl, C₁-C₂haloalkoxy, C₁-C₆alkoxy, mono-(C₁-C₄alkyl)amino, di-(C₁-C₄alkyl)amino, and amino(C₁-C₄ alkyl). In some embodiments, R₂₀ represents 1 substituent chosen from hydroxy, nitro, cyano, amino, halo, C₁-C₆ alkyl, C₁-C₂haloalkyl, C₁-C₂haloalkoxy, C₁-C₆alkoxy, mono-(C₁-C₄alkyl)amino, di-(C₁-C₄alkyl)amino, and amino(C₁-C₄ alkyl).

Some embodiments provide at least one chemical entity chosen from compounds of Formula 11 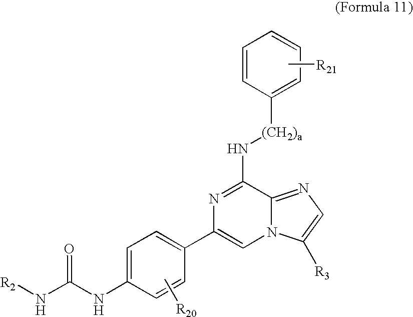
and pharmaceutically acceptable salts, solvates, crystal forms, diastereomers, and prodrugs thereof, wherein R₂, R₃, and R₂₀ are as described for Formula 10, and further wherein a is chosen from 0, 1, 2, and 3; and R₂₁ represents 0 to 3 substituents independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, -di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl.

Some embodiments provide at least one chemical entity chosen from compounds of Formula 12 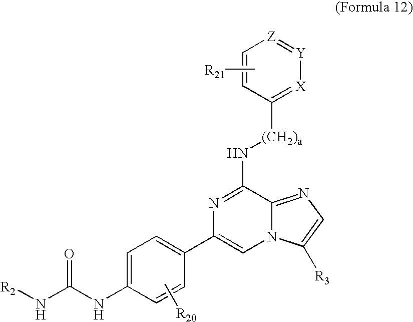
and pharmaceutically acceptable salts, solvates, crystal forms, diastereomers, and prodrugs thereof, wherein R₃, and R₂₀ are as described for Formula 10; and further wherein
   a is chosen from 0, 1, 2, and 3;
   X, Y, and Z are chosen from nitrogen and CH, wherein one and only one of X, Y, and Z is nitrogen;
   R₂ is chosen from
      phenyl,
      substituted phenyl chosen from mono-, di-, and tri-substituted phenyl wherein the substituents are independently chosen from hydroxy, nitro, cyano, amino, halo, C₁-C₆ alkyl, C₁-C₆ alkoxy, C₁-C₂ haloalkyl, C₁-C₂ haloalkoxy, mono-(C₁-C₆ alkyl)amino, di-(C₁-C₆ alkyl)amino, amino(C₁-C₆ alkyl), C₁-C₆alkylthio, mono-(C₁-C₆ alkyl)amino(C₁-C₆ alkyl), di-(C₁-C₆ alkyl)amino(C₁-C₆ alkyl), C₃-C₇ cycloalkyl, piperidinyl, piperazinyl, and morpholinyl,
      pyridyl, and
      substituted pyridyl chosen from mono-, di-, and tri-substituted pyridyl wherein the substituents are independently chosen from hydroxy, nitro, cyano, amino, halo, C₁-C₆ alkyl, C₁-C₆ alkoxy, C₁-C₂ haloalkyl, C₁-C₂ haloalkoxy, mono-(C₁-C₆ alkyl)amino, di-(C₁-C₆ alkyl)amino, amino(C₁-C₆ alkyl), C₁-C₆alkylthio, mono-(C₁-C₆ alkyl)amino(C₁-C₆ alkyl), di-(C₁-C₆ alkyl)amino(C₁-C₆ alkyl), C₃-C₇ cycloalkyl, piperidinyl, piperazinyl, and morpholinyl; and
R₂₁ represents 0 to 4 substituents independently chosen from hydroxy, nitro, cyano, carboxy, amino, sulfonamido, halo, C₁-C₆ alkyl, C₁-C₆alkoxy, C₁-C₆haloalkyl, C₁-C₆haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆alkanoyl, and C₁-C₆alkoxycarbonyl.

Some embodiments provide at least one chemical entity chosen from compounds of Formula 13 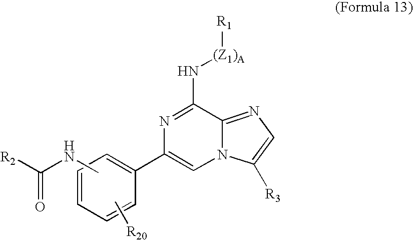
and pharmaceutically acceptable salts, solvates, crystal forms, diastereomers, and prodrugs thereof, wherein R₁, R₃, Z₁, and A are as described in Formula 1; and further wherein R₂ is chosen from
   C₁-C₆ alkyl,
   substituted C₁-C₆ alkyl chosen from mono-, di-, and tri-substituted C₁-C₆ alkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   C₃-C₇ cycloalkyl,
   substituted C₃-C₇ cycloalkyl chosen from mono-, di-, and tri-substituted C₃-C₇ cycloalkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   (C₃-C₇ cycloalkyl)methyl,
   substituted (C₃-C₇ cycloalkyl)methyl chosen from mono-, di-, and tri-substituted (C₃-C₇ cycloalkyl)methyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and
   heterocycloalkyl,
   heterocycloalkyl, substituted heterocycloalkyl chosen from mono-, di-, and tri-substituted heterocycloalkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   (heterocycloalkyl)C₁-C₂ alkyl,
   substituted (heterocycloalkyl)C₁-C₂ alkyl chosen from mono-, di-, and tri-substituted (heterocycloalkyl)C₁-C₂ alkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   C₁-C₆ alkoxy,
   substituted C₁-C₆ alkoxy chosen from mono-, di-, and tri-substituted C₁-C₆ alkoxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   (C₁-C₆ alkoxy)C₁-C₆ alkoxy,
   substituted (C₁-C₆ alkoxy)C₁-C₆ alkoxy chosen from mono-, di-, and tri-substituted (C₁-C₆ alkoxy)C₁-C₆ alkoxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   aryloxy,
   substituted aryloxy chosen from mono-, di-, and tri-substituted aryloxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   phenyl,
   substituted phenyl chosen from mono-, di-, and tri-substituted phenyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   heteroaryl, and s
   ubstituted heteroaryl chosen from mono-, di-, and tri-substituted heteroaryl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl; and
R₂₀ represents 0 to 3 substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

When referring to compounds of Formula 13, in some embodiments, R₂ is chosen from
phenyl substituted with at least one branched C₃-C₆alkyl, and further substituted with 0 to 2 substituents independently chosen from hydroxy, nitro, cyano, amino, halo, C₁-C₆ alkyl, C₁-C₆ alkoxy, C₁-C₂ haloalkyl, C₁-C₂ haloalkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, piperidinyl, piperazinyl, and morpholinyl, and
pyridyl substituted with at least one branched C₃-C₆alkyl, and further substituted with 0 to 2 substituents independently chosen from hydroxy, nitro, cyano, amino, halo, C₁-C₆ alkyl, C₁-C₆ alkoxy, C₁-C₂ haloalkyl, C₁-C₂ haloalkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, piperidinyl, piperazinyl, and morpholinyl.

In some embodiments, R₂₀ represents 1, 2, or 3 substituents independently chosen from hydroxy, nitro, cyano, amino, halo, C₁-C₆ alkyl, C₁-C₂haloalkyl, C₁-C₂haloalkoxy, C₁-C₆alkoxy, mono-(C₁-C₄alkyl)amino, di-(C₁-C₄alkyl)amino, and amino(C₁-C₄ alkyl). In some embodiments, R₂₀ represents 1 substituent chosen from hydroxy, nitro, cyano, amino, halo, C₁-C₆ alkyl, C₁-C₂haloalkyl, C₁-C₂haloalkoxy, C₁-C₆alkoxy, mono-(C₁-C₄alkyl)amino, di-(C₁-C₄alkyl)amino, and amino(C₁-C₄ alkyl).

Some embodiments provide at least one chemical entity chosen from compounds of Formula 14 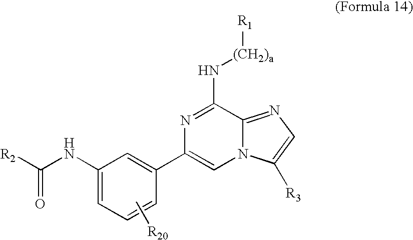
and pharmaceutically acceptable salts, solvates, crystal forms, diastereomers, and prodrugs thereof, wherein R₁, R₂, R₃, and R₂₀ are as described in Formula 13; and further wherein a is chosen from 0 and 1.

When referring to compounds of Formula 14, in some embodiments, R₁ is chosen from
phenyl,
substituted phenyl chosen from mono-, di-, and tri-substituted phenyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆alkanoyl, and C₁-C₆ alkoxycarbonyl,
benzo[d]1,3-dioxolyl,
substituted benzo[d]1,3-dioxolyl chosen from mono-, di-, and tri-substituted benzo[d]1,3-dioxolyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆alkanoyl, and C₁-C₆ alkoxycarbonyl,
pyridyl, and
substituted pyridyl chosen from mono-, di-, and tri-substituted pyridyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆alkanoyl, and C₁-C₆ alkoxycarbonyl.

Some embodiments provide at least one chemical entity chosen from compounds of Formula 15 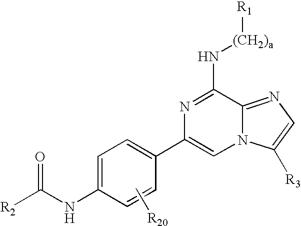
(Formula 15)
   and pharmaceutically acceptable salts, solvates, crystal forms, diastereomers, and prodrugs thereof, wherein R₁, R₂, R₃, and R₂₀ are as described in Formula 13; and further wherein a is chosen from 0 and 1. When referring to compounds of Formula 15, in some embodiments, R₁ is chosen from
phenyl,
substituted phenyl chosen from mono-, di-, and tri-substituted phenyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl,
benzo[d]1,3-dioxolyl,
substituted benzo[d]1,3-dioxolyl chosen from mono-, di-, and tri-substituted benzo[d) 1,3-dioxolyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl,
pyridyl, and
substituted pyridyl chosen from mono-, di-, and tri-substituted pyridyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl.

Some embodiments provide at least one chemical entity chosen from compounds of Formula 16 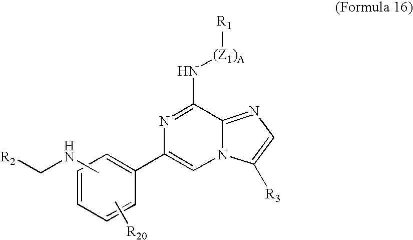
and pharmaceutically acceptable salts, solvates, crystal forms, diastereomers, and prodrugs thereof, wherein R₁, R₃, Z₁, and A are as described in Formula 1; and further wherein R₂ is chosen from
   C₁-C₆ alkyl,
   substituted C₁-C₆ alkyl chosen from mono-, di-, and tri-substituted C₁-C₆ alkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   C₃-C₇ cycloalkyl,
   substituted C₃-C₇ cycloalkyl chosen from mono-, di-, and tri-substituted C₃-C₇ cycloalkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   (C₃-C₇ cycloalkyl)methyl,
   substituted (C₃-C₇ cycloalkyl)methyl chosen from mono-, di-, and tri-substituted (C₃-C₇ cycloalkyl)methyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   heterocycloalkyl,
   substituted heterocycloalkyl chosen from mono-, di-, and tri-substituted heterocycloalkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   (heterocycloalkyl)C₁-C₂ alkyl,
   substituted (heterocycloalkyl)C₁-C₂ alkyl chosen from mono-, di-, and tri-substituted (heterocycloalkyl)C₁-C₂ alkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   C₁-C₆ alkoxy,
   substituted C₁-C₆ alkoxy chosen from mono-, di-, and tri-substituted C₁-C₆ alkoxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   (C₁-C₆ alkoxy)C₁-C₆ alkoxy,
   substituted (C₁-C₆ alkoxy)C₁-C₆ alkoxy chosen from mono-, di-, and tri-substituted (C₁-C₆ alkoxy)C₁-C₆ alkoxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   aryloxy,
   substituted aryloxy chosen from mono-, di-, and tri-substituted aryloxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   phenyl,
   substituted phenyl chosen from mono-, di-, and tri-substituted phenyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   heteroaryl, and
   substituted heteroaryl chosen from mono-, di-, and tri-substituted heteroaryl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl; and
R₂₀ represents 0 to 3 substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₆ alkyl)amino, di-(C₁-C₄ alkyl)amino, and amino(C₁-C₄alkyl).
In some embodiments, R₂₀ represents 1, 2, or 3 substituents independently chosen from hydroxy, nitro, cyano, amino, halo, C₁-C₆ alkyl, C₁-C₂haloalkyl, C₁-C₂haloalkoxy, C₁-C₆alkoxy, mono-(C₁-C₄alkyl)amino, di-(C₁-C₄alkyl)amino, and amino(C₁-C₄ alkyl). In some embodiments, R₂₀ represents 1 substituent chosen from hydroxy, nitro, cyano, amino, halo, C₁-C₆ alkyl, C₁-C₂haloalkyl, C₁-C₂haloalkoxy, C₁-C₆alkoxy, mono-(C₁-C₄alkyl)amino, di-(C₁-C₄alkyl)amino, and amino(C₁-C₄ alkyl).

Some embodiments provide at least one chemical entity chosen from compounds of Formula 17 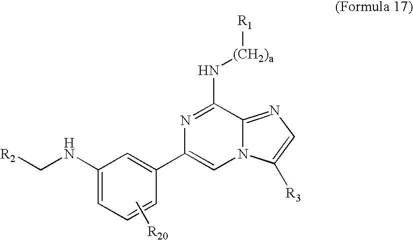
and pharmaceutically acceptable salts, solvates, crystal forms, diastereomers, and prodrugs thereof, wherein R₁, R₂, R₃, and R₂₀ are as described in Formula 16; and further wherein a is chosen from 0 and 1. When referring to compounds of Formula 17, in some embodiments, R₁ is chosen from
   phenyl,
   substituted phenyl chosen from mono-, di-, and tri-substituted phenyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl,
   benzo[d]1,3-dioxolyl,
   substituted benzo[d]1,3-dioxolyl chosen from mono-, di-, and tri-substituted benzo[d]1,3-dioxolyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl,
   pyridyl, and
   substituted pyridyl chosen from mono-, di-, and tri-substituted pyridyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl.

Some embodiments provide at least one chemical entity chosen from compounds of Formula 18 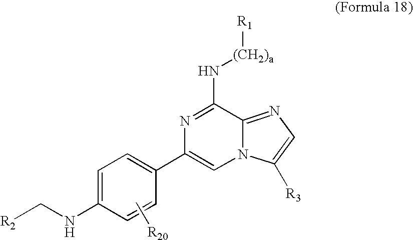
and pharmaceutically acceptable salts, solvates, crystal forms, diastereomers, and prodrugs thereof, wherein R₁, R₂, R₃, and R₂₀, are as described in Formula 16; and further wherein a is chosen from 0 and 1. In some embodiments, R₁ is chosen from
   phenyl,
   substituted phenyl chosen from mono-, di-, and tri-substituted phenyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl,
   benzo[d]1,3-dioxolyl,
   substituted benzo[d]1,3-dioxolyl chosen from mono-, di-, and tri-substituted benzo[d]1,3-dioxolyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl,
   pyridyl, and
   substituted pyridyl chosen from mono-, di-, and tri-substituted pyridyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl.

Some embodiments provide at least one chemical entity chosen from compounds of Formula 19 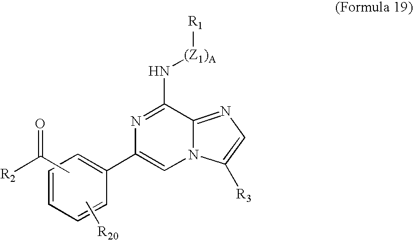
and pharmaceutically acceptable salts, solvates, crystal forms, diastereomers, and prodrugs thereof, wherein R₁, R₃, Z₁, and A are as described in Formula 1; and further wherein R₂ is chosen from
   C₁-C₆ alkyl,
   substituted C₁-C₆ alkyl chosen from mono-, di-, and tri-substituted C₁-C₆ alkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   C₃-C₇ cycloalkyl,
   substituted C₃-C₇ cycloalkyl chosen from mono-, di-, and tri-substituted C₃-C₇ cycloalkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   (C₃-C₇ cycloalkyl)methyl,
   substituted (C₃-C₇ cycloalkyl)methyl chosen from mono-, di-, and tri-substituted (C₃-C₇ cycloalkyl)methyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   heterocycloalkyl,
   substituted heterocycloalkyl chosen from mono-, di-, and tri-substituted heterocycloalkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   (heterocycloalkyl)C₁-C₂ alkyl,
   substituted (heterocycloalkyl)C₁-C₂ alkyl chosen from mono-, di-, and tri-substituted (heterocycloalkyl)C₁-C₂ alkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   C₁-C₆ alkoxy,
   substituted C₁-C₆ alkoxy chosen from mono-, di-, and tri-substituted C₁-C₆ alkoxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   (C₁-C₆ alkoxy)C₁-C₆ alkoxy,
   substituted (C₁-C₆ alkoxy)C₁-C₆ alkoxy chosen from mono-, di-, and tri-substituted (C₁-C₆ alkoxy)C₁-C₆ alkoxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   aryloxy,
   substituted aryloxy chosen from mono-, di-, and tri-substituted aryloxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   phenyl,
   substituted phenyl chosen from mono-, di-, and tri-substituted phenyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
   heteroaryl, and
   substituted heteroaryl chosen from mono-, di-, and tri-substituted heteroaryl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl; and
R₂₀ represents 0 to 3 substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).
In some embodiments, R₂₀ represents 1, 2, or 3 substituents independently chosen from hydroxy, nitro, cyano, amino, halo, C₁-C₆ alkyl, C₁-C₂haloalkyl, C₁-C₂haloalkoxy, C₁-C₆alkoxy, mono-(C₁-C₄alkyl)amino, di-(C₁-C₄alkyl)amino, and amino(C₁-C₄ alkyl). In some embodiments, R₂₀ represents 1 substituent chosen from hydroxy, nitro, cyano, amino, halo, C₁-C₆ alkyl, C₁-C₂haloalkyl, C₁-C₂haloalkoxy, C₁-C₆alkoxy, mono-(C₁-C₄alkyl)amino, di-(C₁-C₄alkyl)amino, and amino(C₁-C₄ alkyl).

Some embodiments provide at least one chemical entity chosen from compounds of Formula 20 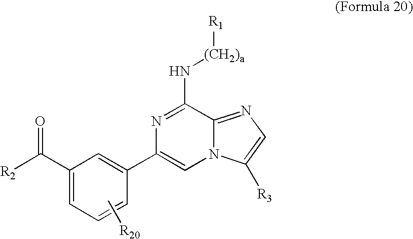
and pharmaceutically acceptable salts, solvates, crystal forms, diastereomers, and prodrugs thereof, wherein R₁, R₂, R₃, and R₂₀, are as described in Formula 19, and further wherein a is chosen from 0 and 1. When referring to compounds of Formula 20, in some embodiments, R₁ is chosen from
   phenyl,
   substituted phenyl chosen from mono-, di-, and tri-substituted phenyl wherein the substituents are chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆alkoxycarbonyl,
   benzo[d]1,3-dioxolyl,
   substituted benzo[d]1,3-dioxolyl chosen from mono-, di-, and tri-substituted benzo[d]1,3-dioxolyl wherein the substituents are chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆alkoxycarbonyl,
   pyridyl, and
   substituted pyridyl chosen from mono-, di-, and tri-substituted pyridyl wherein the substituents are chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆alkox

Some embodiments provide at least one chemical entity chosen from compounds of Formula 21 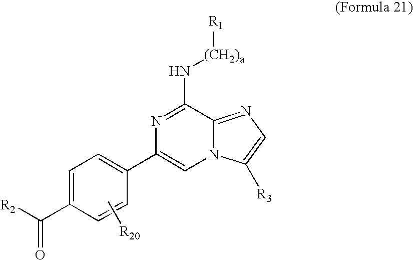
and pharmaceutically acceptable salts, solvates, crystal forms, diastereomers, and prodrugs thereof, wherein R₁, R₂, R₃, and R₂₀, are as described in Formula 19, and further wherein a is chosen from 0 and 1. When referring to compounds of Formula 21, in some embodiments, R₁ is chosen from
   phenyl,
   substituted phenyl chosen from mono-, di-, and tri-substituted phenyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl,
   benzo[d]1,3-dioxolyl,
   substituted benzo[d]1,3-dioxolyl chosen from mono-, di-, and tri-substituted benzo[d]1,3-dioxolyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl,
   pyridyl, and
   substituted pyridyl chosen from mono-, di-, and tri-substituted pyridyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl.

In some embodiments, at least one chemical entity is chosen from
1-(4-Chloro-phenyl)-3-[3-(8-methylamino-imidazo[1,2-a]pyrazin-6-yl)-phenyl]-urea,
1-(4-Chloro-phenyl)-3-[3-(8-phenylamino-imidazo[1,2-a]pyrazin-6-yl)-phenyl]-urea,
1-(4-Chloro-phenyl)-3-{3-[8-(4-chloro-phenylamino)-imidazo[1,2-a]pyrazin-6-yl]-phenyl}-urea,
1-(4-Chloro-phenyl)-3-{3-[8-(3-chloro-phenylamino)-imidazo[1,2-a]pyrazin-6-yl]-phenyl}-urea,
1-(4-Chloro-phenyl)-3-{3-[8-(2-chloro-phenylamino)-imidazo[1,2-a]pyrazin-6-yl]-phenyl}-urea,
1-(4-Chloro-phenyl)-3-{3-[8-(pyridin-3-ylamino)-imidazo[1,2-a]pyrazin-6-yl]-phenyl}-urea,
1-{3-[8-(4-Chloro-benzylamino)-imidazo[1,2-a]pyrazin-6-yl]-phenyl}-3-(4-chloro-phenyl)-urea,
1-{3-[8-(3-Chloro-benzylamino)-imidazo[1,2-a]pyrazin-6-yl]-phenyl}-3-(4-chloro-phenyl)-urea,
1-{4-[8-(4-Chloro-benzylamino)-imidazo[1,2-a]pyrazin-6-yl]-phenyl}-3-(4-chloro-phenyl)-urea,
1-{4-[8-(3-Chloro-benzylamino)-imidazo[1,2-a]pyrazin-6-yl]-phenyl}-3-(4-chloro-phenyl)-urea,
4-(6-{3-[3-(4-Chloro-phenyl)-ureido]-phenyl}-imidazo[1,2-a]pyrazin-8-ylamino)-benzoic acid ethyl ester,
Cyclopropylmethyl-[6-(4-phenoxy-phenyl)-imidazo[1,2-a]pyrazin-8-yl]-amine,
(2-Methoxy-benzyl)-[6-(4-phenoxy-phenyl)-imidazo[1,2-a]pyrazin-8-yl]-amine,
Benzo[1,3]dioxol-5-ylmethyl-[6-(4-phenoxy-phenyl)-imidazo[1,2-a]pyrazin-8-yl]-amine,
[6-(4-Chloromethyl-phenyl)-imidazo[1,2-a]pyrazin-8-yl]-(2-methoxy-benzyl)-amine,
1-{4-[8-(2-Methoxy-benzylamino)-imidazo[1,2-a]pyrazin-6-yl]-phenyl}-3-phenyl-urea,
(2-Methoxy-benzyl)-{6-[4-(4-methoxy-benzylamino)-phenyl]-imidazo[1,2-a]pyrazin-8-yl}-amine,
(2-Methoxy-benzyl)-{6-[3-(4-methoxy-benzylamino)-phenyl]-imidazo[1,2-a]pyrazin-8-yl}-amine,
1-{3-[8-(2-Methoxy-benzylamino)-imidazo[1,2-a]pyrazin-6-yl]-phenyl}-3-phenyl-urea,
1-(2-Chloro-phenyl)-3-{4-[8-(2-methoxy-benzylamino)-imidazo[1,2-a]pyrazin-6-yl]-phenyl}-urea,
1-{4-[8-(2-Methoxy-benzylamino)-imidazo[1,2-a]pyrazin-6-yl]-phenyl}-3-(2-methoxy-phenyl)-urea,
1-{4-[8-(2-Methoxy-benzylamino)-imidazo[1,2-a]pyrazin-6-yl]-phenyl}-3-(3-methoxy-phenyl)-urea,
4-{6-[4-(Piperidine-1-carbonyl)-phenyl]-imidazo[1,2-a]pyrazin-8-ylamino}-benzoic acid ethyl ester,
4-(6-{3-[3-(4-Chloro-phenyl)-ureido]-phenyl}-imidazo[1,2-a]pyrazin-8-ylamino)-benzoic acid ethyl ester,
4-(6-{3-[3-(2-Methylsulfanyl-phenyl)-ureido]-phenyl}-imidazo[1,2-a]pyrazin-8-ylamino)-benzoic acid ethyl ester,
{4-[8-(4-Chloro-phenylamino)-imidazo[1,2-a]pyrazin-6-yl]-phenyl}piperidin-1-yl-methanone,
{4-[8-(2-Chloro-phenylamino)-imidazo[1,2-a]pyrazin-6-yl]-phenyl}-piperidin-1-yl-methanone,
3-Methoxy-N-{4-[8-(2-methoxy-benzylamino)-imidazo[1,2-a]pyrazin-6-yl]-phenyl}-benzamide,
1-(3-Chloro-4-fluoro-phenyl)-3-[3-(8-phenylamino-imidazo[1,2-a]pyrazin-6-yl)-phenyl]-urea,
1-(4-Chloro-phenyl)-3-[3-(8-phenylamino-imidazo[1,2-a]pyrazin-6-yl)-phenyl]-urea,
1-[3-(8-Phenylamino-imidazo[1,2-a]pyrazin-6-yl)-phenyl]-3-(3-trifluoromethyl-phenyl)-urea,
1-(2-Chloro-5-trifluoromethyl-phenyl)-3-[3-(8-phenylamino-imidazof 1,2-a]pyrazin-6-yl)-phenyl]-urea,
1-(4-Chloro-phenyl)-3-{3-[8-(4-chloro-phenylamino)-imidazo[1,2-a]pyrazin-6-yl]-phenyl}-urea,
1-{3-[8-(4-Chloro-phenylamino)-imidazo[1,2-a]pyrazin-6-yl]-phenyl}-3-(3-trifluoromethyl-phenyl)-urea,
1-(3-Chloro-4-fluoro-phenyl)-3-{3-[8-(3-chloro-phenylamino)-imidazo[1,2-a]pyrazin-6-yl]-phenyl}-urea,
1-(4-Chloro-phenyl)-3-{3-[8-(3-chloro-phenylamino)-imidazo[1,2-a]pyrazin-6-yl]-phenyl}-urea,
1-{3-[8-(3-Chloro-phenylamino)-imidazo[1,2-a]pyrazin-6-yl]-phenyl}-3-(3-trifluoromethyl-phenyl)-urea,
1-(3-Chloro-4-fluoro-phenyl)-3-{3-[8-(2-chloro-phenylamino)-imidazo[1,2-a]pyrazin-6-yl]-phenyl}-urea, and
1-(3-Chloro-4-fluoro-phenyl)-3-{3-[8-(2-chloro-phenylamino)-imidazo[1,2-a]pyrazin-6-yl]-phenyl}-urea,
   and pharmaceutically acceptable salts, solvates, crystal forms, diastereomers, and prodrugs thereof.

In some embodiments, at least one chemical entity is chosen from
4-{6-[3-(4-tert-Butyl-benzoylamino)-4-methyl-phenyl]-imidazo[1,2-a]pyrazin-8-ylamino}-benzoic acid,
4-{6-[3-(4-tert-Butyl-benzoylamino)-2-methyl-phenyl]-imidazo[1,2-a]pyrazin-8-ylamino}-benzoic acid,
4-{6-[5-(4-tert-Butyl-benzoylamino)-2-methyl-phenyl]-imidazo[1,2-a]pyrazin-8-ylamino}-benzoic acid,
4-{6-[3-(4-tert-Butyl-benzoylamino)-2-methyl-phenyl]-imidazo[1,2-a]pyrazin-8-ylamino}-benzoic acid ethyl ester, and
4-tert-Butyl-N-{2-methyl-5-[8-(4-sulfamoyl-phenylamino)-imidazo[1,2-a]pyrazin-6-yl]-phenyl}-benzamide,
   and pharmaceutically acceptable salts, solvates, crystal forms, diastereomers, and prodrugs thereof.

Methods for obtaining the compounds described herein are known to those of ordinary skill in the art, suitable procedures being described, for example, in the references cited herein. See, also, U.S. patent application Ser. No. 10/861,791; PCT/US04/18227; U.S. patent application Ser. No. 10/915,696; and PCT/US04/025884, each of which is incorporated by reference herein for all purposes.

As mentioned above, it is believed that the interaction of the chemical entities described herein with one or more kinases results in modulation of the activity of the one or more kinases. Suitable kinases include but are not limited to tyrosine kinases and serine/threonine kinases, which may be classified as including the AGC group (cyclic nucleotide regulated family) of protein kinases, which includes the cyclic nucleotide regulated protein kinase family (e.g., PKA and PKG), the diacylglycerol-activated/phospholipid-dependent family protein kinase C family (e.g., PKC), the PKA and PKC-related family (e.g., RAC and Akt), the kinases that phosphorylate G protein-coupled receptors family, the budding yeast AGC-related protein kinase family, the kinases that phosphorylate ribosomal protein S6 family, the budding yeast DBF2/20 family, the flowering plant PVPK1 protein kinase homolog family, and other AGC related kinase families.

The CaMK (calcium calmodulin dependent) group of protein kinases includes kinases regulated by Ca²⁺/CaM and close relatives family, the KIN1/SNF1/Nim1 family, and other related CaMK related kinase families. The CMGC group (named because it includes the cyclin-dependent kinases) includes the cyclin-dependent kinases (e.g., CDKs) and close relatives family, the ERK (e.g., MAP) kinase family, the glycogen synthase 3 (e.g., GSK3) family, the casein kinase II family, the Clk family and other CMGC kinases.

The PTK group of protein kinases includes protein-tyrosine kinases that may be nonmembrane-spanning or membrane-spanning tyrosine kinases. The PTK group of protein kinases includes the Src family, the Tek/Atk family, the Csk family, the Fes (Fps) family, the Abl family, the Syk/ZAP70 family, the Ttk2/Jak1 family, the Ack family, the focal adhesion kinase (Fak) family, the epidermal growth factor receptor family, the Eph/Elk/Eck receptor family, the Axl family, the Tie/Tek family, the platelet-derived growth factor receptor family, the fibroblast growth factor receptor family, the insulin receptor family, the LTK/ALK family, the Ros/Sevenless family, the Trk/Ror family, the DDR/TKT family, the hepatocyte growth factor receptor family, the nematode Kin15/16 family and other PTK kinase families.

The OPK group (other protein kinases) includes the Polo family, the MEK/STE7 family, the PAK/STE20 family, the MEKK/STE11 family, the NimA family, the wee1/mik1 family, the kinases involved in transcriptional control family, the Raf family, the Activin/TGFb receptor family, the flowering plant putative receptor kinases and close relatives family, the PSK/PTK leucine zipper domain family, the casein kinase I family, the PKN prokaryotic protein kinase family and other OPK protein kinase families. A large number of kinases are found in G. Hardie et al., Protein Kinase Facts Book 0-12-324719-5 (1995).

Accordingly, a method of treating a kinase-implicated disease or condition in a mammal, such as a human, comprises administration to the mammal of a pharmaceutical composition comprising a therapeutically effective amount of at least one chemical entity chosen from compounds of Formula I and pharmaceutically acceptable salts, solvates, crystal forms, diastereomers, and prodrugs thereof, and a pharmaceutically acceptable carrier. As used herein “therapeutically effective” includes alleviation of disease, disease symptoms, preventative, and prophylactic treatment.

Kinases are implicated in a large variety of diseases, as certain mutations in protein kinases can lead to activation of pathways causing, for example, the production of tumors, while other mutations in protein kinases block pathways and prevent a response. Some diseases that are linked to mutations in protein kinases are listed in the KinMutBase database (http://www.uta.fi/imt/bioinfo/KinMutBase/) (Stenberg et al., Nucleic Acids Research, Vol. 28, pp. 369-372, 2000). Diseases caused by protein kinase mutations include X-linked agammaglobulinemia (XLA), and non-insulin dependent diabetes mellitus (NIDDM), and severe combined immunodeficiency (SCID). Mutations related to tumor development have been liked to such diseases as Hirschprung's disease, multiple endocrine neoplasia type 2 (MEN2) a and b, medullary thyroid carcinoma (FMTC), papillary renal carcinoma (HPRC), and Peutz-Jeghers syndrome.

Mutations in growth factor receptor kinases are linked to diseases such as mastocytosis, systemic mast cell disease, piebaldism, hypochondroplasia, thanatophoric dysplasia, and skeletal dysplasia. Other protein kinase-linked diseases include Coffin-Lowry syndrome, congenital insensitivity to pain with anhidrosis (CIPA), hypertension, vascular dysplasia, errors in vascular morphogenesis, and X-linked mental retardation. Mutations in protein kinases have also been linked to neurodegenerative diseases such as amyotrophic lateral sclerosis (ALS) and Alzheimer's disease (AD).

Other diseases associated with protein kinases include Gaucher disease, hypochromic anemia, granulomatous disease, ataxia-telangiectasia, familial hypercholesterolemia, certain types of muscular dystrophy such as Driefuss-Emory type, cystic fibrosis, type 1 hyperlipoproteinemia, Treacher Collins Franceschetti syndrome 1, Tay-Sachs disease, type 1 neurofibromatosis, adenomatous polyposis of the colon, X-linked ichthyosis, and Beckwith-Weidemann Syndrome.

Altered PKA (cyclic AMP-dependent protein kinase) expression is implicated in a variety of disorders and diseases including cancer, thyroid disorders, diabetes, atherosclerosis, and cardiovascular disease. Altered MAP (mitogen-activated protein) kinase expression is implicated in a variety of disease conditions including cancer, inflammation, immune disorders, and disorders affecting growth and development. RTKs (receptor tyrosine kinases), CDKs and STKs (serine/threonine kinases) have all been implicated in a host of pathogenic conditions including, significantly, large number of diverse cancers. Others pathogenic conditions that have been associated with PTKs include, psoriasis, hepatic cirrhosis, diabetes, atherosclerosis, angiogenesis, restinosis, ocular diseases, rheumatoid arthritis and other inflammatory disorders, autoimmune disease, and a variety of renal disorders.

The conditions, diseases and/or disorders that can be affected using at least one chemical entity described herein and compositions comprising such chemical entities include, but are not limited to, psoriasis, cancer (for example, chronic myelogenous leukemia, gastrointestinal stromal tumors, non-small cell lung cancer, breast cancer, ovarian cancer, recurrent ovarian cancer, prostate cancer such as hormonal refractory prostate cancer, kidney cancer, head and neck cancer, or colorectal cancer), immunoregulation (graft rejection), atherosclerosis, rheumatoid arthritis, Parkinson's disease, Alzheimer's disease, diabetes (for example insulin resistance or diabetic retinopathy), septic shock, and the like.

In some embodiments, the condition is cancer. A method of treating cancer comprising administering to a mammal in need thereof a therapeutically effective amount of at least one chemical entity chosen from compounds of Formula I and pharmaceutically acceptable salts, solvates, crystal forms, diastereomers, and prodrugs thereof, and a therapeutically effective amount of an antitumor therapeutic. Treatment with the antitumor therapeutic may be prior to treatment with at least one chemical entity described herein, during treatment, following treatment with the at least one chemical entity, or a combination thereof. Suitable antitumor therapeutics are known, and include a chemotherapeutic agent, for example, chosen from mitomycin C, carboplatin, taxol, cisplatin, paclitaxel, etoposide, and doxorubicin. Radiotherapeutic antitumor agents may also be used, alone or in combination with chemotherapeutic agents.

In another embodiment, pharmaceutical compositions comprising at least one chemical entity chosen from compounds of Formula I and pharmaceutically acceptable salts, solvates, crystal forms, diastereomers, and prodrugs thereof, together with one or more non-toxic, pharmaceutically acceptable carriers and/or diluents and/or adjuvants, and if desired other active ingredients. Such pharmaceutical compositions include packaged pharmaceutical compositions for treating disorders responsive to modulation of kinase activity. A packaged pharmaceutical composition includes a container holding a therapeutically effective amount of at least one chemical entity chosen from compounds of Formula I and pharmaceutically acceptable salts, solvates, crystal forms, diastereomers, and prodrugs thereof, and instructions (e.g., labeling) indicating that the contained composition is to be used for treating a disorder responsive to kinase modulation in the patient. Those of ordinary skill in the art will also recognize a wide variety of non-toxic pharmaceutically acceptable solvents that may be used to prepare solvates of the compounds of the invention, such as water, ethanol, mineral oil, vegetable oil, and dimethylsulfoxide (DMSO).

The chemical entities described herein may be administered orally, topically, parenterally, by inhalation or spray or rectally in dosage unit formulations containing conventional non-toxic pharmaceutically acceptable carriers, adjuvants, and vehicles. In some embodiments, the compounds are administered orally in the form of a pill, capsule, elixir, syrup, lozenge, troche, or the like. The term parenteral as used herein includes subcutaneous injections, intradermal, intravascular (e.g., intravenous), intramuscular, spinal, intrathecal injection or like injection or infusion techniques. The pharmaceutical compositions containing at least one chemical entity chosen from compounds of Formula I and pharmaceutically acceptable salts, solvates, crystal forms, diastereomers, and prodrugs thereof, may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents, and preserving agents in order to provide pharmaceutically elegant and palatable preparations. Tablets may contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate, or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents, which may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil, or coconut oil, or in a mineral oil such as liquid paraffin. The oily suspensions may contain a thickening agent, for example beeswax, hard paraffin, or cetyl alcohol. Sweetening agents, such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent, and one or more preservatives. Suitable dispersing or wetting agents and suspending agents are exemplified by those already mentioned above. Additional excipients, for example sweetening, flavoring, and coloring agents, may also be present.

Suitable pharmaceutical compositions for therapeutic use may also be in the form of oil-in-water emulsions. The oily phase may be a vegetable oil, for example olive oil or arachis oil, or a mineral oil, for example liquid paraffin, or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol, or sucrose. Such formulations may also contain a demulcent, a preservative, and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The chemical entities described herein may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials are cocoa butter and polyethylene glycols.

The chemical entities described herein may be administered parenterally in a sterile medium. The drug, depending on the vehicle and concentration used, can either be suspended or dissolved in the vehicle. Advantageously, adjuvants such as local anesthetics, preservatives, and buffering agents can be dissolved in the vehicle.

For administration to non-human animals, the composition may also be added to the animal feed or drinking water. It is convenient to formulate these animal feed and drinking water compositions so that the animal takes in an appropriate quantity of the composition along with its diet. It is also convenient to present the composition as a premix for addition to the feed or drinking water.

Dosage levels of the order of from about 0.1 milligram to about 140 milligram per kilogram of body weight per day are useful in the treatment of the above-indicated conditions (about 0.5 milligram to about 7 gram per human patient per day). The amount of active ingredient that may be combined with the carrier materials to produce a single dosage form will vary depending upon the host treated and the particular mode of administration. Dosage unit forms will generally contain between from about 1 mg to about 500 milligram of an active ingredient.

Frequency of dosage may also vary depending on the chemical entity used and the particular disease treated. However, for treatment of most disorders, a dosage regimen of 4 times daily or less is used. For the treatment of eating disorders, including obesity, a dosage regimen of 1 or 2 times daily is used. For the treatment of impotence a single dose that rapidly reaches effective concentrations is used. It will be understood, however, that the specific dose level for any particular patient will depend upon a variety of factors including the activity of the specific chemical entity employed, the age, body weight, general health, sex, diet, time of administration, route of administration, and rate of excretion, drug combination and the severity of the particular disease undergoing therapy.

Chemical entities described herein will have at least one pharmacological property. Such properties include, but are not limited to oral bioavailability, low toxicity, low serum protein binding, and desirable in vitro and in vivo half-lives.

Assays may be used to predict these desirable pharmacological properties. Assays used to predict bioavailability include transport across human intestinal cell monolayers, including Caco-2 cell monolayers. Toxicity to cultured hepatocyctes may be used to predict compound toxicity. Penetration of the blood brain barrier of a compound in humans may be predicted from the brain levels of the compound in laboratory animals given the compound intravenously.

Serum protein binding may be predicted from albumin binding assays. Such assays are described in a review by Oravcova, et al. (Journal of Chromatography B 1996, volume 677, pages 1-27).

In general, compound half-life is inversely proportional to the frequency of dosage of a compound. In vitro half-lives of compounds may be predicted from assays of microsomal half-life as described by Kuhnz and Gieschen (Drug Metabolism and Disposition 1998, volume 26, pages 1120-1127).

In another embodiment, the chemical entities are used as probes for the localization of kinases of therapeutic interest, that is, for both in vivo and in vitro identification and isolation the specific proteins to which it binds. A method for identifying a kinase comprises contacting an organism, cell, or preparation comprising the kinase with at least one chemical entity chosen from compounds of Formula I and pharmaceutically acceptable salts, solvates, crystal forms, diastereomers, and prodrugs thereof, and detecting modulation of an activity of the kinase. Suitable methods for detecting kinase modulation are known, for example those described herein.

The invention is further illustrated by the following non-limiting examples.

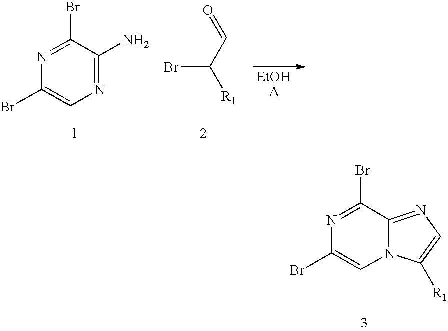

### EXAMPLE 1

### Synthesis of Compounds of Formula 1 (FIG. 1).

6,8-dibromoimidazo[1,2-a]pyrazine (3). A solution of 1.00 equivalents (eq.) of 3,5-dibromo-2-aminopyrazine 1 in ethanol is treated with 2.00 eq. of α-bromo-aldehyde 2 at room temperature (RT) and heated for 48 hours (hr). The solvent is removed under reduced pressure and the residue is triturated with diethyl ether and filtered to give the HBr salt 3.

8-Amino-6-bromoimidazo[1,2-a]pyrazine (4). Procedure 1: A mixture 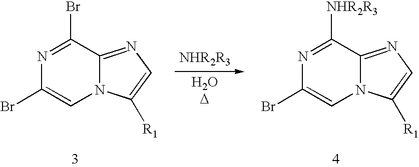
of 1.00 eq. of 6,8-imidazo[1,2-a]pyrazine 3 in 28% ammonia/water solution or 40% aqueous methyl amine is heated to between 80 to 90° C. for 24 hr. The resulting mixture is partitioned between CH₂Cl₂ and H₂O. The aqueous layer is extracted with CH₂Cl₂ and the combined organic extracts are dried over Na₂SO₄. The solvent is removed under reduced pressure and the resulting residue is crystallized from ethanol to yield 4.

Procedure 2: A solution of 1.00 eq. of 6,8-imidazo[1,2-a]pyrazine 3 in N,N-dimethylacetamide is treated with 2.00 eq. of benzylamine and 3.00 eq. of K₂CO₃. The resulting mixture is heated to 100° C. for 24 to 48 hours, cooled to RT and partitioned between H₂O/CH₂Cl₂. The aqueous layer is extracted with CH₂Cl₂ and combined organic extracts are dried over Na₂SO₄. The solvent is removed under reduced pressure and the resulting residue is purified by flash chromatography (3:7 ethyl acetate (EtOAc)/Hexanes) to yield 4.

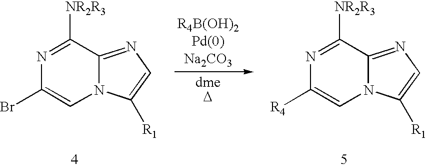

8-Amino-6-aryl-imidazo[1,2-a]pyrazine (5). A mixture of 1.00 eq. of 8-amino-6-bromoimidazo[1,2-a]pyrazine, 3.00 eq. of R₄-substituted boronic acid, and 0.10 eq. of Pd (PPh_{3b})₄, in 6.00 eq. of 1N Na₂CO₃/dme is heated to 90° C. for 24 hr. The mixture is cooled to RT and partitioned between 10% acetic acid (AcOH)/CH₂Cl₂. The aqueous phase is extracted with CH₂Cl₂ and combined extracts are dried over Na₂SO₄. The solvent is removed under reduced pressure and the resulting residue is purified by flash chromatography (1-5% 2M NH₃/methanol/CH₂Cl₂) to yield 5.

### EXAMPLE 2

### Synthesis of Compounds of Formula 1b (FIG. 2).

8-Amino-6-aryl-imidazo[1,2-a]pyrazine (6). A mixture of 1.00 eq. of 8-amino-6-bromoimidazo[1,2-a]pyrazine, 3.00 eq. of R₄-sustituted boronic acid, and 0.10 eq. of Pd (PPh₃)₄, in 4.00 eq. of 1N Na₂CO₃/dme is heated to 90° C. for 24 hr. The mixture is cooled to RT and partitioned between EtOAc/saturated NaHCO₃. The aqueous phase is extracted with EtOAc and the combined extracts are dried over Na₂SO₄. The solvent is removed under reduced pressure and the resulting residue is purified by flash chromatography (1-5% 2M NH₃/methanol/EtOAc) to yield 6.

N-[3-(8-Benzylamino-imidazo[1,2-a]pyrazin-6-yl)-phenyl]-benzamide (8). A solution of 1.00 eq. of 8-amino-6-aryl-imidazo[1,2-a]pyrazine in toluene/DMA is treated dropwise with 1.00 eq. of aryl acid chloride and stirred at RT for 10 hr. The resulting mixture is partitioned between EtOAc/saturated NaHCO₃. The aqueous phase is extracted with EtOAc and the combined extracts are dried over Na₂SO₄. The solvent is removed under reduced pressure and the resulting residue is purified by flash chromatography (1-5% methanol/EtOAc) to yield 8.

N-[3-(8-Benzylamino-imidazo[1,2-a]pyrazin-6-yl)-phenyl]benzenesulfonamide (9). A solution of 1.00 eq. of 8-amino-6-aryl-imidazo[1,2-a]pyrazine in 5% N-methyl morpholine (NMM)/toluene is treated dropwise with 1.1 eq of aryl sulfonyl chloride and heated to 50° C. for 8 h. The solution is cooled to RT and partitioned between EtOAc/saturated NaHCO₃. The aqueous phase is extracted with EtOAc and the combined extracts are dried over Na₂SO₄. The solvent is removed under reduced pressure and the resulting residue is purified by flash chromatography ((1-5% methanol/EtOAc) to yield 9.

1-[3-(8-Benzylamino-imidazo[1,2-a]pyrazin-6-yl)-phenyl]-3-phenyl-urea (10). A solution of 1.00 eq. of 8-amino-6-aryl-imidazo[1,2-a]pyrazine in 5% NMM/toluene is treated dropwise with 1.0 eq of aryl isocyanate and heated to 60° C. for 8 hr. The solution is cooled to RT and partitioned between EtOAc/saturated NaHCO₃. The aqueous phase is extracted with EtOAc and the combined extracts are dried over Na₂SO₄. The solvent is removed under reduced pressure and the resulting residue is purified by flash chromatography (1-5% 2M NH₃/MeOH/EtOAc) to yield 10.

### EXAMPLE 3

The following compounds were prepared in accordance with FIGS. 1 and 2 using the above procedures.

(a) 1-(4-Chloro-phenyl)-3-[3-(8-methylamino-imidazo[1,2-a]pyrazin-6-yl)-phenyl]-urea, MF═C₂₀H₁₇ClN₆O, MW=392.84 Mass Spec m/z (M⁺+1) 393.06.

(b) 1-(4-Chloro-phenyl)-3-[3-(8-phenylamino-imidazo[1,2-a]pyrazin-6-yl)-phenyl]-urea, MF═C₂₅H₁₉ClN₆O MW=454.91 Mass Spec m/z (M⁺+1) 455.04.

(c) 1-(4-Chloro-phenyl)-3-{3-[8-(4-chloro-phenylamino)-imidazo[1,2-a]pyrazin-6-yl]-phenyl}-urea, MF═C₂₅H₁₈Cl₂N₆O, MW=489.36 Mass Spec m/z (M⁺+1) 489.20.

(d) 1-(4-Chloro-phenyl)-3-{3-[8-(3-chloro-phenylamino)-imidazo[1,2-a]pyrazin-6-yl]-phenyl}-urea, MF═C₂₅H₁₈Cl₂N₆O, MW=489.36 Mass Spec m/z (M⁺+1) 489.13.

(e) 1-(4-Chloro-phenyl)-3-{3-[8-(2-chloro-phenylamino)-imidazo[1,2-a]pyrazin-6-yl]-phenyl}-urea, MF═C₂₅H₁₈Cl₂N₆O, MW=489.36 Mass Spec m/z (M⁺+1) 489.04.

(f) 1-(4-Chloro-phenyl)-3-{3-[8-(pyridin-3-ylamino)-imidazo[1,2-a]pyrazin-6-yl]-phenyl}-urea, MF═C₂₄H₁₈ClN₇O, MW=455.90 Mass Spec m/z (M⁺+1) 456.07.

(g) 1-{3-[8-(4-Chloro-benzylamino)-imidazo[1,2-a]pyrazin-6-yl]-phenyl}-3-(4-chloro-phenyl)-urea, MF═C₂₆H₂₀Cl₂N₆O, MW=503.38 Mass Spec m/z (M⁺+1) 503.04.

(h) 1-{3-[8-(3-Chloro-benzylamino)-imidazo[1,2-a]pyrazin-6-yl]-phenyl}-3-(4-chloro-phenyl)-urea, MF═C₂₆H₂₀Cl₂N₆O, MW=503.38 Mass Spec m/z (M⁺+1) 503.01.

(i) 1-{4-[8-(4-Chloro-benzylamino)-imidazo[1,2-a]pyrazin-6-yl]-phenyl}-3-(4-chloro-phenyl)-urea, MF═C₂₆H₂₀Cl₂N₆O, MW=503.38 Mass Spec m/z (M⁺+1) 503.01.
(j) 1-{4-[8-(3-Chloro-benzylamino)-imidazo[1,2-a]pyrazin-6-yl]-phenyl}-3-(4-chloro-phenyl)-urea, MF═C₂₆H₂₀Cl₂N₆O, MW=503.38 Mass Spec m/z (M⁺+1) 503.01.

(k) 4-(6-{3-[3-(4-Chloro-phenyl)-ureido]-phenyl}-imidazo[1,2-a]pyrazin-8-ylamino)-benzoic acid ethyl ester, MF═C₂₈H₂₃ClN₆O₃, MW=526.97 Mass Spec m/z (M⁺+1) 527.05.

(l) Cyclopropylmethyl-[6-(4-phenoxy-phenyl)-imidazo[1,2-a]pyrazin-8-yl]-amine, MF═C₂₂H₂₀N₄O, MW=356.42 Mass Spec m/z (M⁺+1) 357.19.

(m) (2-Methoxy-benzyl)-[6-(4-phenoxy-phenyl)-imidazo[1,2-a]pyrazin-8-yl]-amine, MF═C₂₆H₂₂N₄O₂, MW=422.48 Mass Spec m/z (M⁺+1) 423.19.

(n) Benzo[1,3]dioxol-5-ylmethyl-[6-(4-phenoxy-phenyl)-imidazo[1,2-a]pyrazin-8-yl]-amine, MF═C₂₆H₂₀N₄O₃, MW=436.46 Mass Spec m/z (M⁺+1) 437.18.

(o) [6-(4-Chloromethyl-phenyl)-imidazo[1,2-a]pyrazin-8-yl]-(2-methoxy-benzyl)-amine, MF═C₂₁H₁₉ClN₄O, MW=378.85 Mass Spec m/z (M⁺+1) 379.13.

(p) 1-{4-[8-(2-Methoxy-benzylamino)-imidazo[1,2-a]pyrazin-6-yl]-phenyl}-3-phenyl-urea, MF═C₂₇H₂₄N₆O₂, MW=464.52 Mass Spec m/z (M++I) 465.07.

(q) (2-Methoxy-benzyl)-{6-[4-(4-methoxy-benzylamino)-phenyl]-imidazo[1,2-a]pyrazin-8-yl}-amine, MF═C₂₈H₂₇N₅O₂, MW=465.55 Mass Spec m/z (M⁺+1) 466.10.

(r) (2-Methoxy-benzyl)-{6-[3-(4-methoxy-benzylamino)-phenyl]-imidazo[1,2-a]pyrazin-8-yl}-amine, MF═C₂₈H₂₇N₅O₂, MW=465.55 Mass Spec m/z (M⁺+1) 466.09.

(s) 1-{3-[8-(2-Methoxy-benzylamino)-imidazo[1,2-a]pyrazin-6-yl]-phenyl}-3-phenyl-urea, MF═C₂₇H₂₄N₆O₂, MW=464.52 Mass Spec m/z (M⁺+1) 465.05.

(t) 1-(2-Chloro-phenyl)-3-{4-[8-(2-methoxy-benzylamino)-imidazo[1,2-a]pyrazin-6-yl]-phenyl}-urea, MF═C₂₇H₂₃ClN₆O₂, MW=498.96 Mass Spec m/z (M⁺+1) 499.18.

(u) 1-{4-[8-(2-Methoxy-benzylamino)-imidazo[1,2-a]pyrazin-6-yl]-phenyl}-3-(2-methoxy-phenyl)-urea, MF═C₂₈H₂₆N₆O₃, MW=494.54 Mass Spec m/z (M⁺+1) 495.22.

(v) 1-{4-[8-(2-Methoxy-benzylamino)-imidazo[1,2-a]pyrazin-6-yl]-phenyl}-3-(3-methoxy-phenyl)-urea, MF═C₂₈H₂₆N₆O₃, MW=494.54 Mass Spec m/z (M⁺+1) 495.21.

(w) 4-{6-[4-(Piperidine-1-carbonyl)-phenyl]-imidazo[1,2-a]pyrazin-8-ylamino}-benzoic acid ethyl ester, MF═C₂₇H₂₇N₅O₃, MW=469.54 Mass Spec m/z (M⁺+1) 470.08.

(x) 4-(6-{3-[3-(4-Chloro-phenyl)-ureido]-phenyl}-imidazo[1,2-a]pyrazin-8-ylamino)-benzoic acid ethyl ester, MF═C₂₈H₂₃ClN₆O₃, MW=526.97 Mass Spec m/z (M⁺+1) 527.05.

(y) 4-(6-{3-[3-(2-Methylsulfanyl-phenyl)-ureido]-phenyl}-imidazo[1,2-a]pyrazin-8-ylamino)-benzoic acid ethyl ester, MF═C₂₉H₂₆N₆O₃S, MW=538.62 Mass Spec m/z (M⁺+1) 539.18.

(z) {4-[8-(4-Chloro-phenylamino)-imidazo[1,2-a]pyrazin-6-yl]-phenyl}-piperidin-1-yl-methanone, MF═C₂₄H₂₂ClN₅O, MW=431.92 Mass Spec m/z (M⁺+1) 432.03.

(aa) {4-[8-(2-Chloro-phenylamino)-imidazo[1,2-a]pyrazin-6-yl]-phenyl}-piperidin-1-yl-methanone, MF═C₂₄H₂₂ClN₅O, MW=431.92 Mass Spec m/z (M⁺+1) 432.03.

(b) 3-Methoxy-N-{4-[8-(2-methoxy-benzylamino)-imidazo[1,2-a]pyrazin-6-yl]-phenyl}-benzamide, MF═C₂₈H₂₅N₅O₃, MW=479.53 Mass Spec m/z (M⁺+1) 479.99.

(cc) 1-(3-Chloro-4-fluoro-phenyl)-3-[3-(8-phenylamino-imidazo[1,2-a]pyrazin-6-yl)-phenyl]-urea, MF═C₂₅H₁₈ClFN₆O, MW=472.90 Mass Spec m/z (M⁺+1) 473.01.

(dd) 1-(4-Chloro-phenyl)-3-[3-(8-phenylamino-imidazo[1,2-a]pyrazin-6-yl)-phenyl]-urea, MF═C₂₅H₁₉ClN₆O, MW=454.91 Mass Spec m/z (M⁺+1) 455.04.

(ee) 1-[3-(8-Phenylamino-imidazo[1,2-a]pyrazin-6-yl)-phenyl]-3-(3-trifluoromethyl-phenyl)-urea, MF═C₂₆H₁₉F₃N₆O, MW=488.46 Mass Spec m/z (M⁺+1) 489.01.

(ff) 1-(2-Chloro-5-trifluoromethyl-phenyl)-3-[3-(8-phenylamino-imidazo[1,2-a]pyrazin-6-yl)-phenyl]-urea, MF═C₂₆H₁₈ClF₃N₆O, MW=522.91 Mass Spec m/z (M⁺+1) 523.11.

(gg) 1-(4-Chloro-phenyl)-3-{3-[8-(4-chloro-phenylamino)-imidazo[1,2-a]pyrazin-6-yl]-phenyl}-urea, MF═C₂₅H₁₈Cl₂N₆O, MW=489.36 Mass Spec m/z (M⁺+1) 489.20.

(hh) 1-{3-[8-(4-Chloro-phenylamino)-imidazo[1,2-a]pyrazin-6-yl]-phenyl}-3-(3-trifluoromethyl-phenyl)-urea, MF═C₂₆H₁₈ClF₃N₆O, MW=522.91 Mass Spec m/z (M⁺+1) 523.13.

(ii) 1-(3-Chloro-4-fluoro-phenyl)-3-{3-[8-(3-chloro-phenylamino)-imidazo[1,2-a]pyrazin-6-yl]-phenyl}-urea, MF═C₂₅H₁₇Cl₂FN₆O, MW=507.35 Mass Spec m/z (M⁺+1) 507.13.

(jj) 1-(4-Chloro-phenyl)-3-{3-[8-(3-chloro-phenylamino)-imidazo[1,2-a]pyrazin-6-yl]-phenyl}-urea, MF═C₂₅H₁₈Cl₂N₆O, MW=489.36 Mass Spec m/z (M⁺+1) 489.13.

(kk) 1-{3-[8-(3-Chloro-phenylamino)-imidazo[1,2-a]pyrazin-6-yl]-phenyl}-3-(3-trifluoromethyl-phenyl)-urea, MF═C₂₆H₁₈ClF₃N₆O, MW=522.91 Mass Spec m/z (M⁺+1) 523.12.

(ll) 1-(3-Chloro-4-fluoro-phenyl)-3-{3-[8-(2-chloro-phenylamino)-imidazo[1,2-a]pyrazin-6-yl]-phenyl}-urea, MF═C₂₅H₁₇Cl₂FN₆O, MW=507.35 Mass Spec m/z (M⁺+1) 507.09.

(mm) 4-{6-[3-(4-tert-Butyl-benzoylamino)-4-methyl-phenyl]-imidazo[1,2-a]pyrazin-8-ylamino}-benzoic acid, MF═C₃₁H₂₉N₅O₃, MW=519.59 Mass Spec m/z (M⁺+1) 520.4

(nn) 4-{6-[3-(4-tert-Butyl-benzoylamino)-2-methyl-phenyl]-imidazo[1,2-a]pyrazin-8-ylamino}-benzoic acid, MF═C₃₁H₂₉N₅O₃, MW=519.59 Mass Spec m/z (M⁺+1) 520.2

(oo) 4-{6-[5-(4-tert-Butyl-benzoylamino)-2-methyl-phenyl]-imidazo[1,2-a]pyrazin-8-ylamino}-benzoic acid, MF═C₃₁H₂₉N₅O₃, MW=519.59 Mass Spec m/Z (M⁺+1) 520.1

(pp) 4-{6-[3-(4-tert-Butyl-benzoylamino)-2-methyl-phenyl]-imidazo[1,2-a]pyrazin-8-ylamino}-benzoic acid ethyl ester, MF═C₃₃H₃₃N₅O₃, MW=547.65 Mass Spec m/z (M⁺+1)548.3

(qq) 4-tert-Butyl-N-{2-methyl-5-[8-(4-sulfamoyl-phenylamino)-imidazo[1,2-a]pyrazin-6-yl]-phenyl}-benzamide, MF═C₃₀H₃₀N₆O₃S, MW=554.66 Mass Spec m/z (M⁺+1)—Not ionized

### EXAMPLE 4

A generalized description of the standard AKT-1 Kinase Assay that may be used to evaluate chemical entities is as follows.

In a final reaction volume of 40 microliters (μl), active recombinant N-terminus his-tagged AKT-1/PKBα kinase expressed in Sf21 cells (UBI # 14-276; 50-100 nanogram; 19-38 nanomolar; about 4.5-9 mU) is incubated in 25 mM Tris pH 7.6; 5 mM Beta-glycerophosphate; 2 mM DTT; 100 μM sodium vanadate; 10 mM MgCl₂ in 96-well Pierce Reaci-Bind™ streptavidin-coated high binding capacity coated white plate (Pierce # 15502) coated with saturating amounts of biotinylated Crosstide peptide (UBI #12-385; biotin-KGSGSGRPRTSSFAEG (SEQ ID NO:1); 50 picomoles; about 1.25 μM) and initiated with the addition of 2.5 μCi ³²P-γATP (specific activity 3000 Ci/mmole; 10 mCi/ml; about 21 nM). Compounds are tested initially in duplicate wells for determination of initial IC₅₀ inhibition in half log serial dilutions starting at 100 μM with a final concentration of 2% DMSO. Following a 30 min incubation at 30° C., the reaction is stopped by aspiration and 4×100 μl washes with TBS plus 0.05% Tween-20 prior to addition of 100 μl scintillant and counting in Beckman TopCount instrument. Percent inhibition is calculated as [1−((AVE CPM _{compound}−AVE CPM _{no peptide background})/(AVE CPM _{no compound MAX}−AVE CPM _{no peptide background})))*100]. Staurosporine, a general ATP competitive kinase inhibitor is used as a reference compound and shows an IC50 of approximately 60-100 nM for AKT-1 in the current assay format. Approximate S/N ratios are 8-12× with AVE CPM of Maximum about 15 k and no peptide background about 1.5 K. Improved S/N ratios should be obtainable using higher amounts of either AKT-1 kinase or ³²P-γATP. Cold ATP is not added in current format but has been added at up to 200 μM in the presence of 5 μCi ³²P-γATP resulting in S/N ratios of approximately 5-10×.

### EXAMPLE 5

A generalized description of the standard assay to evaluate modulation of cell growth in soft agar (using cell lines HCT-15 (colon cancer), MiaPaca2 (pancreatic cancer), MCF-7 (breast cancer) and a NIH3T3 clone stably over-expressing transfected myrAkt-1 human gene, for example) is as follows.

Preparation of the agar base layer: A quantity of 500 ml of 2× DMEM (phenol red free, Sigma Cat # D2902) is prepared, and sterile filtered. To that solution is added 10 ml of sodium pyruvate (Gibco, Cat # 11360-070), 10 ml of penicillin/streptomycin (Gibco, Cat# 15140-122), 10 ml of Glutamax (Gibco, cat# 33050-061) and 100 ml of heat-inactivated FBS (Gemini) to make 2× DMEM complete media stock. Two stock concentrations of Sea Plaque low melt agar (Biowhittaker, Cat # 431097), 1%, and 0.6%, are prepared with ultra pure milliQ water, and sterilized by autoclaving. To prepare the agar base layer for a 12-well plate (Falcon # 353042), 6 ml of the 2× DMEM stock is mixed with 6 ml of 1% agar stock, both at 37° C., and 1 ml of the resulting mixture is added to each well of the 12 well plate, 3 hrs prior to setup of top layer.

Top layer with cells and compound for evaluation: Cells at 60-80% confluency (log growth) in T75 are trypsinized with 1 ml of 1× trypsin solution (Gibco), neutralized with 10 ml of 1× DMEM 10% FBS and viable cells counted

| | |
|---|---|
| XHOI-J1 | |
| 5′-CCG CTC GAG ACT GAA GTG GAC CCC | (SEQ ID NO:2) |
| ACA CAT-3′ | |
| J1-KPNI | |
| 5′-CGG GOT ACC TTA TTT TAA AAG TGC | (SEQ ID NO:3) |
| TTC AAA-3′ | |

JAK1 PCR products are cloned into the pFastBac HTh expression vector (Gitco) via the Xho I and Kpn I sites. The JAK1 plasmid is then transformed into competent DHlOBac cells (Gibco), and the recombinant baculovirus produced prepared for transfection into Sf9 insect cells.
JAK2

The kinase domain of human JAK2 is amplified from U937mRNA using the polymerase chain reaction with the following primers:

| | |
|---|---|
| SALI-jk2 | |
| 5′-ACG CGT CGA CGG TGC CTT TGA AGA | (SEQ ID NO:4) |
| CCG GGA T-3′ | |
| jk2-NOTI | |
| 5′-ATA GTT TAG CGG CCG CTC AGA ATG | (SEQ ID NO:5) |
| AAG GTC ATT T-3′ | |
| JAK2 | |

PCR products are cloned into the pFastBac HTc expression vector (Gibco) via the Sal I and Not I sites. The JAK2 plasmid is then transformed into competent DHlOBac cells (Gitco), and the recombinant baculovirus produced prepared for transfection into Sf9 insect cells.
JAK3

The kinase domain of humanJAK3 is amplified from U937mRNA using the polymerase chain reaction with the following primers:

| | |
|---|---|
| XHOI-J3 | |
| 5′-CCG GTC GAG TAT GCC TGC CAA GAC | (SEQ ID NO:6) |
| CCC ACG-3′ | |
| J3-KPNI | |
| 5′-CGG GGT ACC CTA TGA AAA GGA CAG | (SEQ ID NO:7) |
| GGA GTG-3′ | |

using a hemocytometer via trypan blue exclusion. A working stock of 2.5×10⁴ cells/ml is prepared in 1× DMEM 10% FBS. A 15 ml centrifuge tube is prepared for each concentration of compound tested in duplicate wells of a 12 well plate. The following are added in order: 1 ml of 2× DMEM stock at 37° C.; compound at 2× final desired concentration (using 4 microliter volume from a 1000× concentrated dilution series in 100% DMSO); followed by 2,500 cells (using 100 microliters of 1×10⁴ cell/ml working stock), and finally 1 ml of 0.6% agar stock at 37° C. Following careful mixing, 1 ml each is added to duplicate wells of the 12-well plate. The plate is then placed in a 37° C., 5% CO₂, humidified incubator for 10 to 14 days and read. Rapid difflusion of CPD throughout top and bottom agar layer results in final drug concentration of 1×.

Counting Colonies: After 10 days of incubation, the plates are removed from the incubator for photography and colony counting. Each well is scanned using an eyepiece with a micrometer guide and 5× phase optics. Colonies 50 micrometer or greater in diameter are scored as positive. Duplicate wells are averaged and percent inhibition calculated using number of colonies in no compound control wells as 100%.

All compounds described in Examples 1-3 were tested in accordance with the protocols of Examples 4-5 and determined to exhibit an IC₅₀ value less than or equal to 25 micromolar.

### EXAMPLE 6

### Inhibition of JAK Tyrosine Kinase and Tyrosine Kinase 2 Activity

Chemical entites as disclosed herein exhibit less than 50% inhibition of JAK Tyrosine Kinase and Tyrosine Kinase 2 when tested in the following assay. For screening purposes, compounds are diluted in 96 well plates at a concentration of 10 micromolar. Chemical entities are tested at a concentration of I micromolar. Plates are warmed at 37° C. for 30 minutes before assay.

JAK kinase domains are produced as follows:

JAK1

The kinase domain of humanJAKI is amplified from U937mRNA using the polymerase chain reaction with the following primers:

JAK3 PCR products are cloned into the pFastBac HTh expression vector (GiLco) via the Xho I and Kpn I sites. The JAK3 plasmid is then transformed into competent DHlOBac cells (Gibco), and the recombinant baculovirus produced prepared for transfection into Sf9 insect cells.

TYK2

The kinase domain of humanTYK2 is amplified from A549 mRNA using the polymerase chain reaction with the following primers:

| | |
|---|---|
| HT2EK | |
| 5′-GGA GCA CTC GAG ATG GTA GCA CAC | (SEQ ID NO:8) |
| AAC CAG GTG-3′ | |
| ITY2.2R | |
| 5′-GGA GCA GGA ATT COG GCG CTG COG | (SEQ ID NO:9) |
| GTC AAA TCT GG-3′ | |

TYK2 PCR products are cloned into pBlueBacHis2A (Invitrogen) via the EcoRI site. The recombinant TYK2 baculovirus produced is prepared for transfected into Sf9 insect cells.
Large Scale Production Of Kinase Domains

Baculovirus preparations from each of the JAK family members are infected into five litres of High Five cells (Invitrogen) grown in High Five serum free medium (Invitrogen) to a cell density of approximately 1-2×10⁶ cells/ml.

Cells are infected with virus at a MOI of 0.8-3.0. Cells are harvested and lysed. JAK kinase domains are purified by affinity chromatography on a Probond (Invitrogen) nickel chelate affinity column.

Assay Protocols

Kinase assays are performed in a 96 well capture-based ELISA assay, using approximately 1.5 ug of affinity purified PTK domain in the presence of 50 mM HEPES, pH 7.5, 10 mM MgCl₂, 150 mM NaCl and 10-20,uM ATP. The biotinylated substrate biotin-EGPWLEEEEEAYGWMDF-NH₂ (SEQ ID NO:10) (final 30 concentration HIM) is used as substrate, and tyrosine phosphorylation is quantitated following transfer to an avidin coated ELISA plate using peroxidase-linked anti-phospho-tyrosine antibody PY20.

Inhibitors are added to the assays fifteen minutes prior to the addition of ATP. Inhibitors are added in aqueous DMSO, with DMSO concentrations never exceeding 1%.

The cellular assays of Example 6 are performed as follows: Cell suspensions are prepared by harvesting cells from culture. Cell used in this test should be in later log phase growth and high viability. Cells are diluted in correct growth medium to 1.1× final concentration (from 50,000 5 cell/ml to 200,000 cell/ml, depending on cell line). 90 uL is added to samples, diluted in PBS to 10× final concentration in flat-bottom 96-well plates (DOLL). After incubation for 40 hr in 37° C. 5% CO₂ incubator, MTT 5 mg/ml (in PBS, filter sterile) 20 ul per well is added. The plates are returned to incubator for another 6 hours. Lysis Buffer (10% SDS, 0.01N HCl) 100 ul per well is added and the plate put back in incubator overnight. The plate is then read at 590 nm.

### EXAMPLE 7

### Btk Assays

A generalized procedure for one standard biochemical Btk Kinase Assay used to test compounds of formula 1 is as follows.

A master mix minus Btk enzyme is prepared containing 1× Cell Signaling kinase buffer, 0.5 uM Promega PTK Biotinylated peptide substrate 2, and 0.01% BSA. A master mix plus Btk enzyme is prepared containing 1× Cell Signaling kinase buffer, 0.5 uM PTK Biotinylated peptide substrate 2, 0.01% BSA, and 100 ng/well (0.06 mU/well) BTK enzyme. A solution of 200 uM ATP is prepared. A quantity of 1.25 uL of compounds/DMSO is transferred to a 96-well ½ area Costar polystyrene plate. A quantity of 18.75 uL of master mix minus enzyme and master mix plus enzyme is transferred to appropriate wells in 96-well ½ area costar polystyrene plate. To that mixture is added 5 uL of 200 uM ATP to 96-well ½ area Costar polystyrene plate for final ATP concentration of 40 uM. The reaction is allowed to incubate for 1 hour at room temperature. The reaction is stopped with Perkin Elmer IX detection buffer containing 30 mM EDTA, 20 nM SA-APC, and 1 nM PT66 Ab. The plate is read using time-resolved fluorescence with a Perkin Elmer Envision using excitation filter 330 nm, emission filter 665 nm, and 2^{nd} emission filter 615 nm. IC₅₀ values are subsequently calculated.

Another generalized procedure for a standard cellular Btk Kinase Assay used to test compounds disclosed in this application is as follows.

Ramos cells are incubated at a density of 0.5×10⁷ cells/ml in the presence of test compound for 1 hr at 37° C. Cells are then stimulated by incubating with 10 ug/ml anti-human IgM F(ab)₂ for 5 minutes at 37° C. Cells are pelleted, lysed, and a protein assay is performed on the cleared lysate. Equal protein amounts of each sample are subject to SDS-PAGE and western blotting with either anti-phosphoBtk(Tyr223) antibody (Cell Signaling Technology #3531) to assess Btk autophosphorylation or an anti-Btk antibody (BD Transduction Labs #611116) to control for total amounts of Btk in each lysate.

### EXAMPLE 8

### EphB₄ Assays

The following is a general procedure for a standard biochemical assay for EphB₄ Kinase Activty

Materials:

96-well, ½ area flat bottom, white polystyrene plates are purchased from Costar, cat #3693.

The cytoplasmic domain of recombinant EphB₄ kinase (amino acids 596-987, Homo sapiens EphB₄, GENBANK Accession No. AY056047.1) with a C-terminal 6× his tag is purified from Sf9 cells. Purity of >95% is assessed by Sypro-Ruby staining of SDS gels.

PTK Biotinylated Peptide Substrate 2, is purchased from Promega, cat #V288A; Lot # 740360.

LANCE Eu-W1024 labeled anti-phosphotyrosine antibody (PT66) is purchased from Perkin-Elmer, cat #AD0068; Lot #109144. Kinase Buffer is purchased from Cell Signaling cat #9802.

Method:

Dilutions of compounds are made in 100% DMSO at 20× the final desired concentration. Compounds in 100% DMSO are transferred (1.25 μL) to the 96 well assay plate. A 18.75 μL volume of master mix containing the final concentrations (in 25 ul) of 0.01% BSA, IX Cell Signaling Kinase Buffer, 0.5 μM PTK Biotinylated Peptide Substrate 2, and 60 ng/well of EphB₄ kinase is added to all wells, except the four negative control wells (which contain no kinase), and mixed. To initiate the reaction, 5 μL of 550 uM ATP is added to each well. (Final Concentration of ATP=110 μM). The reactions are incubated for 1 hour at room temperature (RT). After incubation a quantity of 8.35 μL of a 4×SA-APC Detection Mix is added to each well. The final concentration of Eu-labelled PT66 antibody is 1 nM and the SA-APC is 20 nM (based on the SA moiety). The reaction plates are incubated at RT for at least 15 minutes after SA-APC Detection Mix addition. The reaction plates are read on an Envision plate reader (Perkin-Elmer) with 605 nm Excitation and 605 nm and 640 nm Emission wavelengths. Values are corrected for the fluorescence in the absence of enzyme and inhibition curves are fit to the data using a Logit curve-fitting algorithm. IC₅₀ values are determined from these inhibition curves.

The following cell-based assay may also used to determine the effect of compounds on EphB₄ activity.

HEK293 cells stably expressing V5-epitope tagged EphB₄ are grown to ˜75% confluency, and then incubated for 1 hr at 37° C. in low serum media (Optimem) containing test compound. Cells are stimulated for 10 minutes at 37° C. with 500 ng/ml EphrinB₂/Fc chimera and 50 ng/ml goat-anti-human IgG (FC specific) in low serum media containing test compound. Cells are washed in ice-cold PBS, lysed, and protein assays are performed on the cleared lysates. Equal protein amounts of each sample are subjected to SDS-PAGE and western blotting with either an anti-phosphotyrosine antibody or an anti-V5 antibody to control for total amounts of v5-tagged EphB₄ in each lysate.

Chemical entities disclosed herein are tested in the biochemical and cellular assay of Example 7 and are determined to exhibit an IC₅₀ value less than or equal to 1 micromolar in the biochemical assay and 10 micromolar in the cellular assay. In another embodiment, chemical entities are tested in the biochemical and cellular assay of Example 8 and are determined to exhibit an IC₅₀ value less than or equal to I micromolar in the biochemical assay and 10 micromolar in the cellular assay.

While some embodiments have been shown and described, various modifications and substitutions may be made thereto without departing from the spirit and scope of the invention. Accordingly, it is to be understood that the present invention has been described by way of illustration and not limitations.

1. At least one chemical entity chosen from compounds of Formula I: 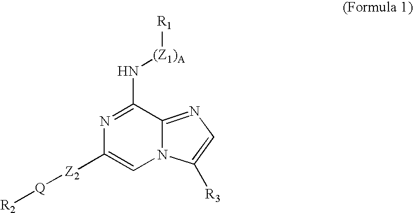
and pharmaceutically acceptable salts, solvates, crystal forms, diastereomers, and prodrugs thereof, wherein
A is chosen from 0 and 1;
Z₁ is chosen from 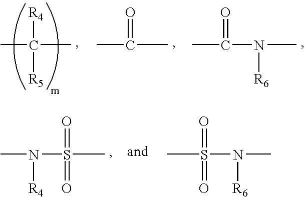
wherein
each occurrence of R₄ and R₅ is independently chosen from hydrogen, C₁-C₆ alkyl, sulfonamido, and halo;
m is chosen from 0, 1, 2, and 3; and
R₆ is chosen from
hydrogen,
C₁-C₆ alkyl,
phenyl,
substituted phenyl chosen from mono-, di-, and tri-substituted phenyl wherein the substitutents are independently chosen from hydroxy, nitro, cyano, amino, halo, C₁-C₆ alkyl, C₁-C₆ haloalkyl, C₁-C₆ haloalkoxy, C₁-C₆ alkoxy, (C₁-C₆ alkoxy)C₁-C₆ alkoxy, mono-(C₁-C₆ alkyl)amino, di-(C₁-C₆ alkyl)amino, and amino(C₁-C₆ alkyl),
heteroaryl, and
substituted heteroaryl chosen from mono-, di-, and tri-substituted heteroaryl wherein the substitutents are independently chosen from hydroxy, nitro, cyano, amino, halo, C₁-C₆ alkyl, C₁-C₆ haloalkyl, C₁-C₆ haloalkoxy, C₁-C₆ alkoxy, (C₁-C₆ alkoxy)C₁-C₆ alkoxy, mono-(C₁-C₆ alkyl)amino, di-(C₁-C₆ alkyl)amino, and amino(C₁-C₆ alkyl);
R₁ is chosen from
hydrogen,
C₁-C₇ alkyl,
C₁-C₆ alkoxy,
C₃-C₇ cycloalkyl,
(C₃-C₇ cycloalkyl)methyl,
heterocycloalkyl,
(heterocycloalkyl)C₁-C₂ alkyl,
sulfonamido,
(C₁-C₆ alkoxy)C₁-C₆ alkoxy,
mono-(C₁-C₆ alkyl)amino,
di-(C₁-C₆ alkyl)amino,
mono-(C₁-C₆ alkyl)amino(C₁-C₆ alkyl),
di(C₁-C₆ alkyl)amino(C₁-C₆ alkyl),
phenyl,
substituted phenyl chosen from mono-, di-, and tri-substituted phenyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
phenyl fused to a 5- to 7-membered heterocycloalkyl ring containing 1 or 2 heteroatoms chosen from N, O, and S,
substituted phenyl fused to a 5- to 7-membered heterocycloalkyl ring containing 1 or 2 heteroatoms chosen from N, O, and S, or heteroaryl, chosen from mono-, di-, and tri-substituted phenyl fused to a 5- to 7-membered heterocycloalkyl ring containing 1 or 2 heteroatoms chosen from N, O, and S, or heteroaryl, wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
heteroaryl, and
substituted heteroaryl chosen from mono-, di-, and tri-substituted heteroaryl, wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl;
Z₂ is chosen from
substituted phenylene chosen from mono-, di-, and tri-substituted phenylene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl),
pyridylidene,
substituted pyridylidene chosen from mono-, di-, and tri-substituted pyridylidene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl), and
substituted naphthylidene chosen from mono-, di-, and tri-substituted naphthylene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl);
Q is chosen from 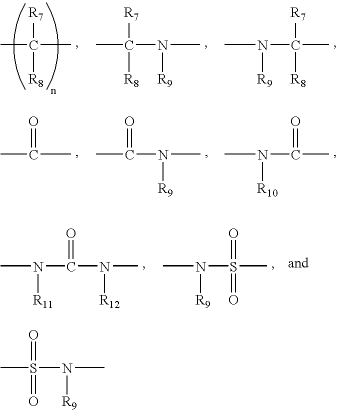
wherein
each occurrence of R₇ and R₈ is independently chosen from C₁-C₆ alkyl, sulfonamido, and halo;
n is chosen from 0, 1, 2, and 3; and
R₉-R₁₂ are each independently chosen from
hydrogen,
C₁-C₆ alkyl,
phenyl,
substituted phenyl chosen from mono-, di-, and tri-substituted phenyl wherein the substituents are independently chosen from hydroxy, nitro, cyano, amino, halo, C₁-C₆ alkyl, C₁-C₆ alkoxy, (C₁-C₆ alkyloxy)C₁-C₆ alkoxy, C₁-C₆ perfluoroalkyl, C₁-C₆ perfluoroalkoxy, mono-(C₁-C₆ alkyl)amino, di(C₁-C₆ alkyl)amino, and amino(C₁-C₆ alkyl),
heteroaryl, and
substituted heteroaryl chosen from mono-, di-, and tri-substituted phenyl wherein the substituents are independently chosen from hydroxy, nitro, cyano, amino, halo, C₁-C₆ alkyl, C₁-C₆ alkoxy, (C₁-C₆ alkyloxy)C₁-C₆ alkoxy, C₁-C₆ perfluoroalkyl, C₁-C₆ perfluoroalkoxy, mono-(C₁-C₆ alkyl)amino, di(C₁-C₆ alkyl)amino, and amino(C₁-C₆ alkyl);
R₂ is chosen from
C₁-C₇ alkyl,
substituted C₁-C₇ alkyl chosen from mono-, di-, and tri-substituted C₁-C₇ alkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
C₃-C₇ cycloalkyl,
substituted C₃-C₇ cycloalkyl chosen from mono-, di-, and tri-substituted C₃-C₇ cycloalkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
heterocycloalkyl,
substituted heterocycloalkyl chosen from mono-, di-, and tri-substituted heterocycloalkyl, wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
C₁-C₆ alkoxy,
substituted C₁-C₆ alkoxy chosen from mono-, di-, and tri-substituted C₁-C₆ alkoxy, wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
aryloxy,
substituted aryloxy chosen from mono-, di-, and tri-substituted aryloxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
phenyl,
substituted phenyl chosen from mono-, di-, and tri-substituted phenyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
heteroaryl, and
substituted heteroaryl chosen from mono-, di-, and tri-substituted heteroaryl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl; and
R₃ is chosen from hydrogen, C₁-C₇ alkyl, C₃-C₇ cycloalkyl, (C₃-C₇ cycloalkyl)C₁-C₂ alkyl, heterocycloalkyl, and (heterocycloalkyl)C₁-C₂ alkyl.

2. At least one chemical entity of claim 1 wherein R₂-Q- is halo-C₁-C₄ alkyl.

3. At least one chemical entity of claim 1 wherein R₂-Q- is chosen from
phenoxy and
substituted phenoxy chosen from mono-, di-, and tri-substituted phenoxy wherein the substituents are independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

4. At least one chemical entity of claim 1 wherein
R₆ is chosen from hydrogen, C₁-C₆ alkyl, and phenyl;
R₁ is chosen from
phenyl,
substituted phenyl chosen from mono-, di-, and tri-substituted phenyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
benzo[d]1,3-dioxolyl,
substituted benzo[d]1,3-dioxolyl chosen from mono-, di-, and tri-substituted benzo[d]1,3-dioxolyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
pyridyl,
substituted pyridyl chosen from mono-, di-, and tri-substituted pyridyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
pyrimidinyl, and
substituted pyrimidinyl chosen from mono-, di-, and tri-substituted pyrimidinyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl;
Z₂ is chosen from
substituted phenylene chosen from mono-, di-, and tri-substituted phenylene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl),
pyridylidene,
substituted pyridylidene chosen from mono-, di-, and tri-substituted pyridylidene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl), and
substituted naphthylidene chosen from mono-, di-, and tri-substituted naphthylene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl);
R₂-Q- is chosen from
chloro-C₁-C₄ alkyl,
phenoxy, and
substituted phenoxy chosen from mono-, di-, and tri-substituted phenoxy wherein the substituents are independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄alkyl)amino, di-(C₁-C₄alkyl)amino, and amino(C₁-C₄alkyl).

5. At least one chemical entity of claim 1 wherein
R₆ is chosen from hydrogen, C₁-C₆ alkyl, and phenyl;
R₁ is chosen from
phenyl,
substituted phenyl chosen from mono-, di-, and tri-substituted phenyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
benzo[d]1,3-dioxolyl,
substituted benzo[d]1,3-dioxolyl chosen from mono-, di-, and tri-substituted benzo[d]1,3-dioxolyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
pyridyl,
substituted pyridyl chosen from mono-, di-, and tri-substituted pyridyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
pyrimidinyl, and
substituted pyrimidinyl chosen from mono-, di-, and tri-substituted pyrimidinyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl;
Z₂ is chosen from
substituted phenylene chosen from mono-, di-, and tri-substituted phenylene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl),
pyridylidene,
substituted pyridylidene chosen from mono-, di-, and tri-substituted pyridylidene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl), and
substituted naphthylidene chosen from mono-, di-, and tri-substituted naphthylene with substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl);
Q is chosen from 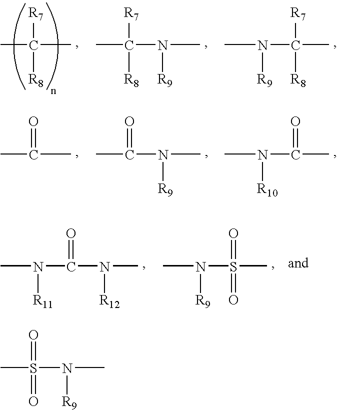
wherein
each occurrence of R₇ and R₈ is independently chosen from C₁-C₆ alkyl, sulfonamido, and halo, and n is chosen from 0, 1, 2, and 3; and
R₉-R₁₂ are each independently chosen from hydrogen, C₁-C₆ alkyl, and phenyl;
R₂ is chosen from
C₃-C₇ cycloalkyl,
substituted C₃-C₇ cycloalkyl chosen from mono-, di-, and tri-substituted C₃-C₇ cycloalkyl wherein the substituents are independently chosen from hydroxy, cyano, amino, halo, C₁-C₄ alkyl, C₁-C₄ alkoxy, C₁-C₂ haloalkyl, C₁-C₂ haloalkoxy, mono-(C₁-C₄ alkyl)amino, and di-(C₁-C₄ alkyl)amino,
(C₃-C₇ cycloalkyl)methyl,
substituted (C₃-C₇ cycloalkyl)methyl chosen from mono-, di-, and tri-substituted (C₃-C₇ cycloalkyl)methyl wherein the substituents are independently chosen from hydroxy, cyano, amino, halo, C₁-C₄ alkyl, C₁-C₄ alkoxy, C₁-C₂ haloalkyl, C₁-C₂ haloalkoxy, mono-(C₁-C₄ alkyl)amino, and di-(C₁-C₄ alkyl)amino,
heterocycloalkyl,
substituted heterocycloalkyl chosen from mono-, di-, and tri-substituted heterocycloalkyl, wherein the substituents are independently chosen from hydroxy, cyano, amino, halo, C₁-C₄ alkyl, C₁-C₄ alkoxy, C₁-C₂ haloalkyl, C₁-C₂ haloalkoxy, mono-(C₁-C₄ alkyl)amino, and di-(C₁-C₄ alkyl)amino,
(heterocycloalkyl)C₁-C₂ alkyl,
substituted (heterocycloalkyl)C₁-C₂ alkyl chosen from mono-, di-, and tri-substituted (heterocycloalkyl)C₁-C₂ alkyl, wherein the substituents are independently chosen from hydroxy, cyano, amino, halo, C₁-C₄ alkyl, C₁-C₄ alkoxy, C₁-C₂ haloalkyl, C₁-C₂ haloalkoxy, mono-(C₁-C₄ alkyl)amino, and di-(C₁-C₄ alkyl)amino,
C₁-C₆ alkoxy,
substituted C₁-C₆ alkoxy chosen from mono-, di-, and tri-substituted C₁-C₆ alkoxy, wherein the substituents are independently chosen from hydroxy, cyano, amino, halo, C₁-C₄ alkyl, C₁-C₄ alkoxy, C₁-C₂ haloalkyl, C₁-C₂ haloalkoxy, mono-(C₁-C₄ alkyl)amino, and di-(C₁-C₄ alkyl)amino,
aryloxy,
substituted aryloxy chosen from mono-, di-, and tri-substituted aryloxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
phenyl,
substituted phenyl chosen from mono-, di-, and tri-substituted phenyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
pyridyl,
substituted pyridyl chosen from mono-, di-, and tri-substituted pyridyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
pyrimidinyl, and
substituted pyrimidinyl chosen from mono-, di-, and tri-substituted pyrimidinyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,

6. At least one chemical entity of claim 1 wherein Q is chosen from 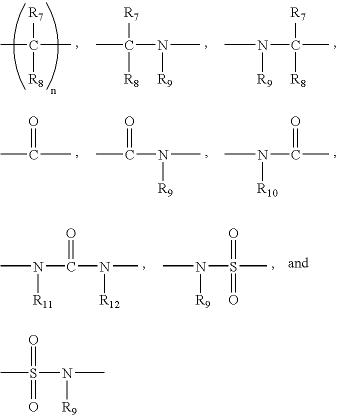
wherein
each occurrence of R₇ and R₈ is independently chosen from C₁-C₆ alkyl, sulfonamido, and halo; and n is chosen from 1 and 2; and
R₉-R₁₂ are each independently chosen from hydrogen, C₁-C₆ alkyl, and phenyl.

7. At least one chemical entity of claim 1 chosen from compounds of Formula 2: 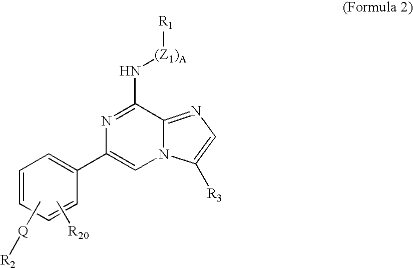
and pharmaceutically acceptable salts, solvates, crystal forms, diastereomers, and prodrugs thereof, wherein
R₂₀ represents 1, 2, or 3 substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

8. At least one chemical entity of claim 7 wherein R₂₀ represents one substituent chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

9. At least one chemical entity of claim 7 wherein R₁ is chosen from
phenyl,
substituted phenyl chosen from mono-, di-, and tri-substituted phenyl wherein the substituents are chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
benzo[d]1,3-dioxolyl,
substituted benzo[d]1,3-dioxolyl chosen from mono-, di-, and tri-substituted benzo[d]1,3-dioxolyl wherein the substituents are chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl,
pyridyl, and
substituted pyridyl chosen from mono-, di-, and tri-substituted pyridyl wherein the substituents are chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, (C₁-C₆ alkoxy)C₁-C₆alkoxy, C₁-C₆ alkylthio, mono-(C₁-C₆ alkyl)amino, di-(C₁-C₆ alkyl)amino, amino(C₁-C₆ alkyl), mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, and C₁-C₆ alkoxycarbonyl.

10. At least one chemical entity of claim 7 chosen from compounds of Formula 3 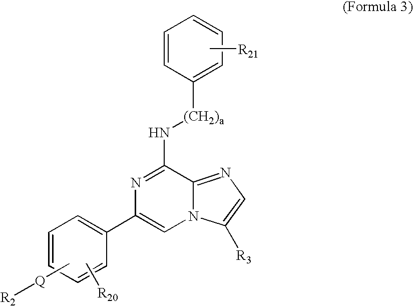
and pharmaceutically acceptable salts, solvates, crystal forms, diastereomers, and prodrugs thereof, wherein
a is chosen from 0, 1, 2 and 3;
R₂₁ represents 0 to 3 substituents independently chosen from hydroxy, nitro, cyano, carboxy, amino, sulfonamido, halo, C₁-C₆ alkyl, C₁-C₂ haloalkyl, C₁-C₂ haloalkoxy, C₁-C₆ alkoxy, mono-(C₁-C₆ alkyl)amino, di-(C₁-C₆ alkyl)amino, mono-(C₁-C₆ alkyl)amino(C₁-C₆ alkyl), di(C₁-C₆ alkyl)amino(C₁-C₆ alkyl), C₂-C₆alkanoyl, and C₁-C₆alkoxycarbonyl.

11. At least one chemical entity of claim 10 wherein R₂₀ represents one substituent chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

12. At least one chemical entity of claim 7 chosen from compounds of Formula 4 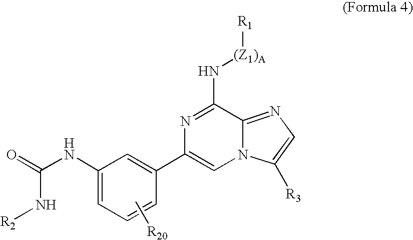
and pharmaceutically acceptable salts, solvates, crystal forms, diastereomers, and prodrugs thereof

13. At least one chemical entity of claim 12 wherein R₂₀ represents 1 substituent chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

14. At least one chemical entity of claim 12 chosen from compounds of Formula 5 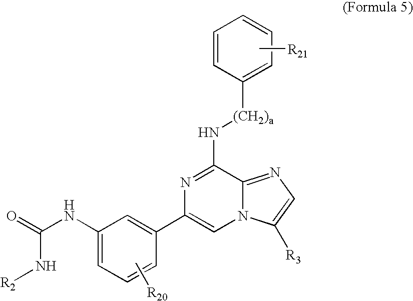
and pharmaceutically acceptable salts, solvates, crystal forms, diastereomers, and prodrugs thereof, wherein
a is chosen from 0, 1, 2 and 3;
R₂₁ represents 0 to 3 substituents independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl, and
R₂ is chosen from
phenyl,
substituted phenyl chosen from mono-, di-, and tri-substituted phenyl wherein the substituents are independently chosen from hydroxy, nitro, cyano, amino, halo, C₁-C₆ alkyl, C₁-C₆ alkoxy, C₁-C₂ haloalkyl, C₁-C₂ haloalkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, piperidinyl, piperazinyl, and morpholinyl,
pyridyl, and
substituted pyridyl chosen from mono-, di-, and tri-substituted pyridyl wherein the substituents are independently chosen from hydroxy, nitro, cyano, amino, halo, C₁-C₆ alkyl, C₁-C₆ alkoxy, C₁-C₂ haloalkyl, C₁-C₂ haloalkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, piperidinyl, piperazinyl, and morpholinyl.

15. At least one chemical entity of claim 14 wherein R₂₀ represents one substituent chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

16. At least one chemical entity of claim 12 chosen from compounds of Formula 6 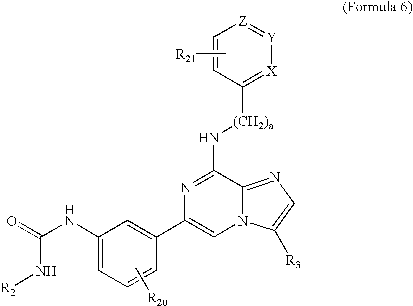
and pharmaceutically acceptable salts, solvates, crystal forms, diastereomers, and prodrugs thereof, wherein
X, Y, and Z are chosen from nitrogen and CH, wherein one and only one of X, Y, and Z is nitrogen;
R₂₁ represents 0, 1, 2, or 3 substituents independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl;
R₂ is chosen from
C₁-C₆ alkyl,
substituted C₁-C₆ alkyl chosen from mono-, di-, and tri-substituted C₁-C₆ alkyl wherein the substituents are chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
C₃-C₇ cycloalkyl,
substituted C₃-C₇ cycloalkyl alkyl chosen from mono-, di-, and tri-substituted C₃-C₇ cycloalkyl wherein the substituents are chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
heterocycloalkyl,
substituted heterocycloalkyl chosen from mono-, di-, and tri-substituted heterocycloalkyl wherein the substituents are chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
C₁-C₆ alkoxy,
substituted C₁-C₆ alkoxy chosen from mono-, di-, and tri-substituted C₁-C₆ alkoxy wherein the substituents are chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
aryloxy,
substituted aryloxy chosen from mono-, di-, and tri-substituted aryloxy wherein the substituents are chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
phenyl,
substituted phenyl chosen from mono-, di-, and tri-substituted phenyl wherein the substituents are chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
heteroaryl, and
substituted heteroaryl chosen from mono-, di-, and tri-substituted heteroaryl wherein the substituents are chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl.

17. At least one chemical entity of claim 16 wherein R₂₀ represents one substituent chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

18. At least one chemical entity of claim 16 wherein
R₂ is chosen from
phenyl,
substituted phenyl chosen from mono-, di-, and tri-substituted phenyl wherein the substituents are independently chosen from hydroxy, nitro, cyano, amino, halo, C₁-C₆ alkyl, C₁-C₆ alkoxy, C₁-C₂ haloalkyl, C₁-C₂ haloalkoxy, mono-(C₁-C₆ alkyl)amino, di-(C₁-C₆ alkyl)amino, amino(C₁-C₆ alkyl), C₁-C₆ alkylthio, di-(C₁-C₆ alkyl)amino(C₁-C₆ alkyl), mono-(C₁-C₆ alkyl)amino(C₁-C₆ alkyl), C₃-C₇ cycloalkyl, piperidinyl, piperazinyl, and morpholinyl,
pyridyl, and
substituted pyridyl chosen from mono-, di-, and tri-substituted pyridyl wherein the substituents are independently chosen from hydroxy, nitro, cyano, amino, halo, C₁-C₆ alkyl, C₁-C₆ alkoxy, C₁-C₂ haloalkyl, C₁-C₂ haloalkoxy, mono-(C₁-C₆ alkyl)amino, di-(C₁-C₆ alkyl)amino, amino(C₁-C₆ alkyl), C₁-C₆ alkylthio, di-(C₁-C₆ alkyl)amino(C₁-C₆ alkyl), mono-(C₁-C₆ alkyl)amino(C₁-C₆ alkyl), C₃-C₇ cycloalkyl, piperidinyl, piperazinyl, and morpholinyl.

19. At least one chemical entity of claim 7 chosen from compounds of Formula 7 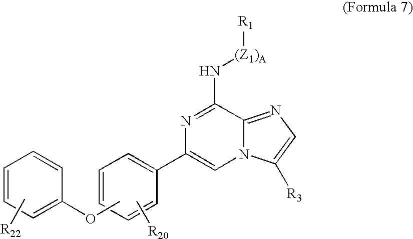
and pharmaceutically acceptable salts, solvates, crystal forms, diastereomers, and prodrugs thereof wherein
R₁ is chosen from
C₃-C₇ cycloalkyl,
phenyl,
substituted phenyl chosen from mono-, di-, and tri-substituted phenyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆haloalkoxy, mono-(C₁-C₆ alkyl)amino, di-(C₁-C₆ alkyl)amino, di-(C₁-C₆ alkyl)amino(C₁-C₆ alkyl), mono-(C₁-C₆ alkyl)amino(C₁-C₆ alkyl), C₂-C₆ alkanoyl, and C₁-C₆ alkoxycarbonyl,
benzo[d]1,3-dioxolyl,
substituted benzo[d]1,3-dioxolyl chosen from mono-, di-, and tri-substituted benzo[d]1,3-dioxolyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆haloalkoxy, mono-(C₁-C₆ alkyl)amino, di-(C₁-C₆ alkyl)amino, di-(C₁-C₆ alkyl)amino(C₁-C₆ alkyl), mono-(C₁-C₆ alkyl)amino(C₁-C₆ alkyl), C₂-C₆ alkanoyl, and C₁-C₆ alkoxycarbonyl,
pyridyl, and
substituted pyridyl chosen from mono-, di-, and tri-substituted pyridyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆haloalkoxy, mono-(C₁-C₆ alkyl)amino, di-(C₁-C₆ alkyl)amino, di-(C₁-C₆ alkyl)amino(C₁-C₆ alkyl), mono-(C₁-C₆ alkyl)amino(C₁-C₆ alkyl), C₂-C₆ alkanoyl, and C₁-C₆ alkoxycarbonyl;
R₂₂ represents 0 to 4 substituents independently chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

20. At least one chemical entity of claim 19 wherein R₂₀ represents one substituent chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

21. At least one chemical entity of claim 19 chosen from compounds of Formula 8 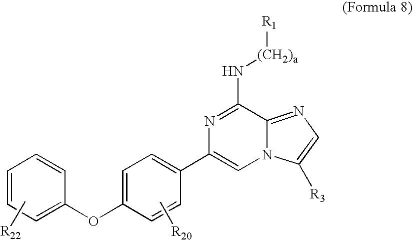
and pharmaceutically acceptable salts, solvates, crystal forms, diastereomers, and prodrugs thereof, wherein
a is chosen from 0 and 1.

22. At least one chemical entity of claim 21 wherein R₂₀ represents one substituent chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

23. At least one chemical entity of claim 19 chosen from compounds of Formula 9 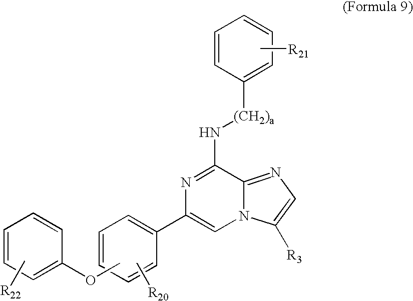
and pharmaceutically acceptable salts, solvates, crystal forms, diastereomers, and prodrugs thereof, wherein
R₂₁ represents 0 to 3 substituents independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl.

24. At least one chemical entity of claim 23 wherein R₂₀ represents one substituent chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

25. At least one chemical entity of claim 7 chosen from compounds of Formula 10 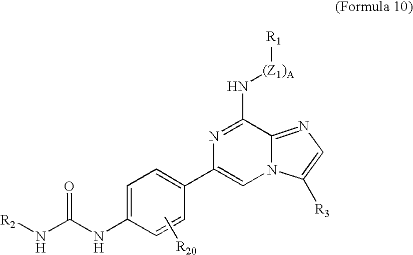
and pharmaceutically acceptable salts, solvates, crystal forms, diastereomers, and prodrugs thereof, wherein
R₂ is chosen from
C₁-C₆ alkyl,
substituted C₁-C₆ alkyl chosen from mono-, di-, and tri-substituted C₁-C₆ alkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, di-(C₁-C₆ alkyl)amino(C₁-C₆ alkyl), mono-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
C₃-C₇ cycloalkyl,
substituted C₃-C₇ cycloalkyl alkyl chosen from mono-, di-, and tri-substituted C₃-C₇ cycloalkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, di-(C₁-C₆ alkyl)amino(C₁-C₆ alkyl), mono-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
heterocycloalkyl,
substituted heterocycloalkyl chosen from mono-, di-, and tri-substituted heterocycloalkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, di-(C₁-C₆ alkyl)amino(C₁-C₆ alkyl), mono-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
C₁-C₆ alkoxy,
substituted C₁-C₆ alkoxy chosen from mono-, di-, and tri-substituted C₁-C₆ alkoxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, di-(C₁-C₆ alkyl)amino(C₁-C₆ alkyl), mono-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
aryloxy,
substituted aryloxy chosen from mono-, di-, and tri-substituted aryloxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, di-(C₁-C₆ alkyl)amino(C₁-C₆ alkyl), mono-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
phenyl,
substituted phenyl chosen from mono-, di-, and tri-substituted phenyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, di-(C₁-C₆ alkyl)amino(C₁-C₆ alkyl), mono-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
heteroaryl, and
substituted heteroaryl chosen from mono-, di-, and tri-substituted heteroaryl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, di-(C₁-C₆ alkyl)amino(C₁-C₆ alkyl), mono-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl

26. At least one chemical entity of claim 25 wherein R₂₀ represents one substituent chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

27. At least one chemical entity of claim 25 chosen from compounds of Formula 11 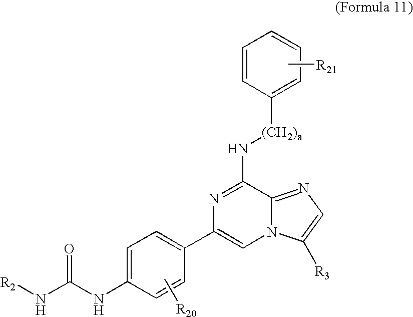
and pharmaceutically acceptable salts, solvates, crystal forms, diastereomers, and prodrugs thereof, wherein
a is chosen from 0, 1, 2, and 3; and
R₂₁ represents 0 to 3 substituents independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl.

28. At least one chemical entity of claim 27 wherein R₂₀ represents one substituent chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

29. At least one chemical entity of claim 25 chosen from compounds of Formula 12 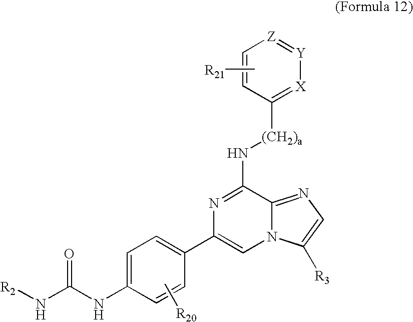
and pharmaceutically acceptable salts, solvates, crystal forms, diastereomers, and prodrugs thereof, wherein
a is chosen from 0, 1, 2, and 3;
X, Y, and Z are chosen from nitrogen and CH, wherein one and only one of X, Y, and Z is nitrogen; and
R₂ is chosen from
phenyl,
substituted phenyl chosen from mono-, di-, and tri-substituted phenyl wherein the substituents are independently chosen from hydroxy, nitro, cyano, amino, halo, C₁-C₆ alkyl, C₁-C₆ alkoxy, C₁-C₂ haloalkyl, C₁-C₂ haloalkoxy, mono-(C₁-C₆ alkyl)amino, di-(C₁-C₆ alkyl)amino, amino(C₁-C₆ alkyl), C₁-C₆alkylthio, mono-(C₁-C₆ alkyl)amino(C₁-C₆ alkyl), di-(C₁-C₆ alkyl)amino(C₁-C₆ alkyl), C₃-C₇ cycloalkyl, piperidinyl, piperazinyl, and morpholinyl,
pyridyl, and
substituted pyridyl chosen from mono-, di-, and tri-substituted pyridyl wherein the substituents are independently chosen from hydroxy, nitro, cyano, amino, halo, C₁-C₆ alkyl, C₁-C₆ alkoxy, C₁-C₂ haloalkyl, C₁-C₂ haloalkoxy, mono-(C₁-C₆ alkyl)amino, di-(C₁-C₆ alkyl)amino, amino(C₁-C₆ alkyl), C₁-C₆alkylthio, mono-(C₁-C₆ alkyl)amino(C₁-C₆ alkyl), di-(C₁-C₆ alkyl)amino(C₁-C₆ alkyl), C₃-C₇ cycloalkyl, piperidinyl, piperazinyl, and morpholinyl

30. At least one chemical entity of claim 29 wherein R₂₀ represents one substituent chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

31. At least one chemical entity of claim 7 chosen from compounds of Formula 13 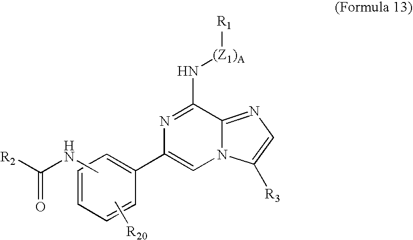
and pharmaceutically acceptable salts, solvates, crystal forms, diastereomers, and prodrugs thereof, wherein
R₂ is chosen from
C₁-C₆ alkyl,
substituted C₁-C₆ alkyl chosen from mono-, di-, and tri-substituted C₁-C₆ alkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
C₃-C₇ cycloalkyl,
substituted C₃-C₇ cycloalkyl chosen from mono-, di-, and tri-substituted C₃-C₇ cycloalkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
heterocycloalkyl,
substituted heterocycloalkyl chosen from mono-, di-, and tri-substituted heterocycloalkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
C₁-C₆ alkoxy,
substituted C₁-C₆ alkoxy chosen from mono-, di-, and tri-substituted C₁-C₆ alkoxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
aryloxy,
substituted aryloxy chosen from mono-, di-, and tri-substituted aryloxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
phenyl,
substituted phenyl chosen from mono-, di-, and tri-substituted phenyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
heteroaryl, and
substituted heteroaryl chosen from mono-, di-, and tri-substituted heteroaryl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl

32. At least one chemical entity of claim 31 wherein R₂₀ represents one substituent chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

33. At least one chemical entity of claim 31 wherein R₂ is chosen from
phenyl substituted with at least one branched C₃-C₆alkyl, and further substituted with 0 to 2 substituents independently chosen from hydroxy, nitro, cyano, amino, halo, C₁-C₆ alkyl, C₁-C₆ alkoxy, C₁-C₂ haloalkyl, C₁-C₂ haloalkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, piperidinyl, piperazinyl, and morpholinyl, and
pyridyl substituted with at least one branched C₃-C₆alkyl, and further substituted with 0 to 2 substituents independently chosen from hydroxy, nitro, cyano, amino, halo, C₁-C₆ alkyl, C₁-C₆ alkoxy, C₁-C₂ haloalkyl, C₁-C₂ haloalkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, piperidinyl, piperazinyl, and morpholinyl.

34. At least one chemical entity of claim 31 chosen from compounds of Formula 14 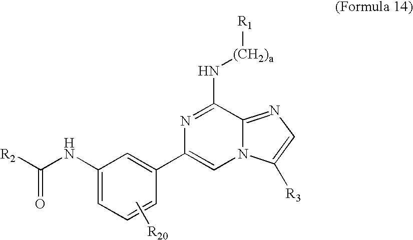
and pharmaceutically acceptable salts, solvates, crystal forms, diastereomers, and prodrugs thereof, wherein
a is chosen from 0 and 1.

35. At least one chemical entity of claim 34 wherein R₂₀ represents one substituent chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

36. At least one chemical entity of claim 34 wherein R₁ is chosen from
phenyl,
substituted phenyl chosen from mono-, di-, and tri-substituted phenyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆alkanoyl, and C₁-C₆ alkoxycarbonyl,
benzo[d]1,3-dioxolyl,
substituted benzo[d]1,3-dioxolyl chosen from mono-, di-, and tri-substituted benzo[d]1,3-dioxolyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆alkanoyl, and C₁-C₆ alkoxycarbonyl,
pyridyl, and
substituted pyridyl chosen from mono-, di-, and tri-substituted pyridyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆alkanoyl, and C₁-C₆ alkoxycarbonyl.

37. At least one chemical entity of claim 31 chosen from compounds of Formula 15 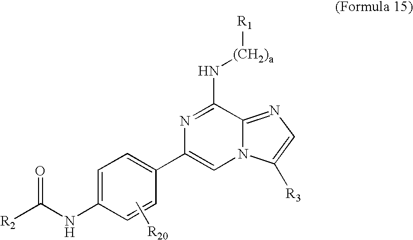
and pharmaceutically acceptable salts, solvates, crystal forms, diastereomers, and prodrugs thereof, wherein
a is chosen from 0 and 1.

38. At least one chemical entity of claim 37 wherein R₂₀ represents one substituent chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

39. At least one chemical entity of claim 37 wherein, R₁ is chosen from
phenyl,
substituted phenyl chosen from mono-, di-, and tri-substituted phenyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl,
benzo [d]1,3-dioxolyl,
substituted benzo[d]1,3-dioxolyl chosen from mono-, di-, and tri-substituted benzo[d]1,3-dioxolyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl,
pyridyl, and
substituted pyridyl chosen from mono-, di-, and tri-substituted pyridyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl.

40. At least one chemical entity of claim 7 chosen from compounds of Formula 16 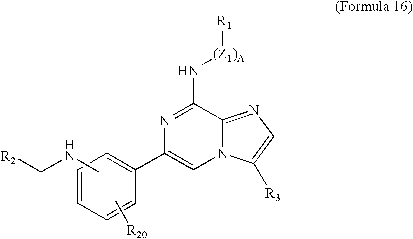
and pharmaceutically acceptable salts, solvates, crystal forms, diastereomers, and prodrugs thereof, wherein
R₂ is chosen from
C₁-C₆ alkyl,
substituted C₁-C₆ alkyl chosen from mono-, di-, and tri-substituted C₁-C₆ alkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
C₃-C₇ cycloalkyl,
substituted C₃-C₇ cycloalkyl chosen from mono-, di-, and tri-substituted C₃-C₇ cycloalkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
heterocycloalkyl,
substituted heterocycloalkyl chosen from mono-, di-, and tri-substituted heterocycloalkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
C₁-C₆ alkoxy,
substituted C₁-C₆ alkoxy chosen from mono-, di-, and tri-substituted C₁-C₆ alkoxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
aryloxy,
substituted aryloxy chosen from mono-, di-, and tri-substituted aryloxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
phenyl,
substituted phenyl chosen from mono-, di-, and tri-substituted phenyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
heteroaryl, and
substituted heteroaryl chosen from mono-, di-, and tri-substituted heteroaryl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl.

41. At least one chemical entity of claim 40 wherein R₂₀ represents one substituent chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

42. At least one chemical entity of claim 40 chosen from compounds of Formula 17 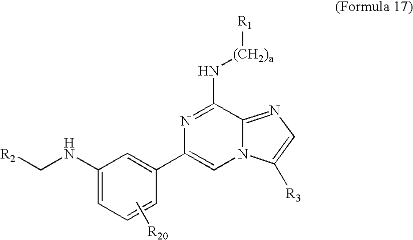
and pharmaceutically acceptable salts, solvates, crystal forms, diastereomers, and prodrugs thereof, wherein
); and
a is chosen from 0 and 1.

43. At least one chemical entity of claim 42 wherein R₂₀ represents one substituent chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

44. At least one chemical entity of claim 42 wherein R₁ is chosen from
phenyl,
substituted phenyl chosen from mono-, di-, and tri-substituted phenyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl,
benzo[d]1,3-dioxolyl,
substituted benzo[d]1,3-dioxolyl chosen from mono-, di-, and tri-substituted benzo[d]1,3-dioxolyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl,
pyridyl, and
substituted pyridyl chosen from mono-, di-, and tri-substituted pyridyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl.

45. At least one chemical entity of claim 40 chosen from compounds of Formula 18 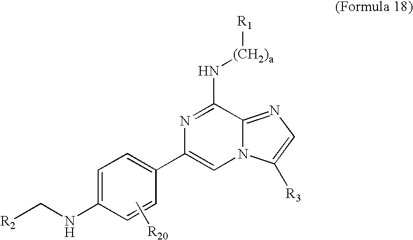
and pharmaceutically acceptable salts, solvates, crystal forms, diastereomers, and prodrugs thereof, wherein
a is chosen from 0 and 1.

46. At least one chemical entity of claim 45 wherein R₂₀ represents one substituent chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

47. At least one chemical entity of claim 44 wherein R₁ is chosen from
phenyl,
substituted phenyl chosen from mono-, di-, and tri-substituted phenyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl,
benzo[d]1,3-dioxolyl,
substituted benzo[d]1,3-dioxolyl chosen from mono-, di-, and tri-substituted benzo[d]1,3-dioxolyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl,
pyridyl, and
substituted pyridyl chosen from mono-, di-, and tri-substituted pyridyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl.

48. At least one chemical entity of claim 7 chosen from compounds of Formula 19 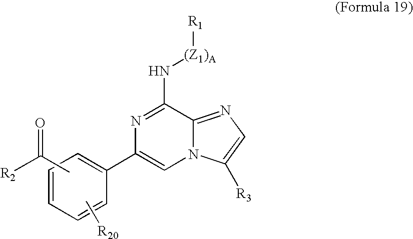
and pharmaceutically acceptable salts, solvates, crystal forms, diastereomers, and prodrugs thereof, wherein
R₂ is chosen from
C₁-C₆ alkyl,
substituted C₁-C₆ alkyl chosen from mono-, di-, and tri-substituted C₁-C₆ alkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
C₃-C₇ cycloalkyl,
substituted C₃-C₇ cycloalkyl chosen from mono-, di-, and tri-substituted C₃-C₇ cycloalkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
heterocycloalkyl,
substituted heterocycloalkyl chosen from mono-, di-, and tri-substituted heterocycloalkyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
C₁-C₆ alkoxy,
substituted C₁-C₆ alkoxy chosen from mono-, di-, and tri-substituted C₁-C₆ alkoxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
aryloxy,
substituted aryloxy chosen from mono-, di-, and tri-substituted aryloxy wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
phenyl,
substituted phenyl chosen from mono-, di-, and tri-substituted phenyl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl,
heteroaryl, and
substituted heteroaryl chosen from mono-, di-, and tri-substituted heteroaryl wherein the substituents are independently chosen from hydroxy, nitro, cyano, amino, halo, C₁-C₆ alkyl, C₁-C₆ alkoxy, C₁-C₆ haloalkyl, C₁-C₆ haloalkoxy, (C₁-C₆ alkoxy)C₁-C₆ alkoxy, mono-(C₁-C₆ alkyl)amino, di-(C₁-C₆ alkyl)amino, amino(C₁-C₆ alkyl), C₁-C₆ alkylthio, mono-(C₁-C₆ alkyl)amino(C₁-C₆ alkyl), di-(C₁-C₆ alkyl)amino(C₁-C₆ alkyl), C₃-C₇ cycloalkyl, C₂-C₆ alkanoyl, C₁-C₆ alkoxycarbonyl, and heterocycloalkyl.

49. At least one chemical entity of claim 48 wherein R₂₀ represents one substituent chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

50. At least one chemical entity of claim 48 chosen from compounds of Formula 20 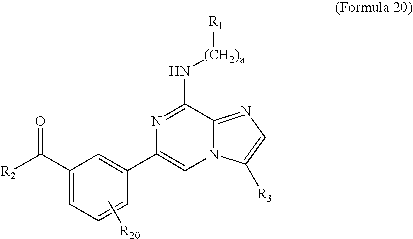
and pharmaceutically acceptable salts, solvates, crystal forms, diastereomers, and prodrugs thereof, wherein
a is chosen from 0 and 1.

51. At least one chemical entity of claim 50 wherein R₂₀ represents one substituent chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

52. At least one chemical entity of claim 50 wherein R₁ is chosen from
phenyl,
substituted phenyl chosen from mono-, di-, and tri-substituted phenyl wherein the substituents are chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆alkoxycarbonyl,
benzo[d]1,3-dioxolyl,
substituted benzo[d]1,3-dioxolyl chosen from mono-, di-, and tri-substituted benzo[d]1,3-dioxolyl wherein the substituents are chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆alkoxycarbonyl,
pyridyl, and
substituted pyridyl chosen from mono-, di-, and tri-substituted pyridyl wherein the substituents are chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆alkox

53. At least one chemical entity of claim 48 chosen from compounds of Formula 21 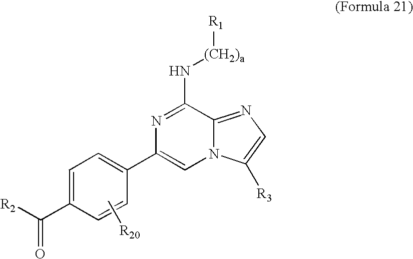
and pharmaceutically acceptable salts, solvates, crystal forms, diastereomers, and prodrugs thereof, wherein
a is chosen from 0 and 1.

54. At least one che mical entity of claim 53 wherein R₂₀ r epresents one substituent chosen from hydroxy, nitro, cyano, amino, halo, C₁-C₆ alkyl, C₁-C₂ haloalkyl, C₁-C₂ haloalkoxy, C₁-C₆ alkoxy, mono-(C₁-C₄ alkyl)amino, di-(C₁-C₄ alkyl)amino, and amino(C₁-C₄ alkyl).

55. At least one chemical entity of claim 53 wherein R₁ is chosen from
phenyl,
substituted phenyl chosen from mono-, di-, and tri-substituted phenyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl,
benzo[d]1,3-dioxolyl,
substituted benzo[d]1,3-dioxolyl chosen from mono-, di-, and tri-substituted benzo[d]1,3-dioxolyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl,
pyridyl, and
substituted pyridyl chosen from mono-, di-, and tri-substituted pyridyl wherein the substituents are independently chosen from hydroxy, nitro, cyano, carboxy, amino, sulfonamido, halo, C₁-C₆ alkyl, C₁-C₆ alkoxy, C₁-C₆ haloalkyl, C₁-C₆ haloalkoxy, mono-(C₁-C₆ alkyl)amino, di-(C₁-C₆ alkyl)amino, mono-(C₁-C₆ alkyl)amino(C₁-C₆ alkyl), di-(C₁-C₆ alkyl)amino(C₁-C₆ alkyl), C₂-C₆ alkanoyl, and C₁-C₆ alkoxycarbonyl.

56. At least one chemical entity of claim 1 chosen from
4-{6-[3-(4-tert-Butyl-benzoylamino)-4-methyl-phenyl]-imidazo[1,2-a]pyrazin-8-ylamino}-benzoic acid;
4-{6-[3-(4-tert-Butyl-benzoylamino)-2-methyl-phenyl]-imidazo[1,2-a]pyrazin-8-ylamino}-benzoic acid;
4-{6-[5-(4-tert-Butyl-benzoylamino)-2-methyl-phenyl]-imidazo[1,2-a]pyrazin-8-ylamino}-benzoic acid;
4-{6-[3-(4-tert-Butyl-benzoylamino)-2-methyl-phenyl]-imidazo[1,2-a]pyrazin-8-ylamino}-benzoic acid ethyl ester;
4-tert-Butyl-N-{2-methyl-5-[8-(4-sulfamoyl-phenylamino)-imidazo[1,2-a]pyrazin-6-yl]-phenyl}-benzamide;
and pharmaceutically acceptable salts, solvates, crystal forms, diastereomers, and prodrugs thereof.

57. At least one chemical entity according to claim 1, wherein in an in vitro assay of kinase modulation, the at least one chemical entity exhibits an IC₅₀ value less than or equal to 25 micromolar.

58. At least one chemical entity according to claim 1, wherein in an in vitro assay of modulation of soft agar growth, the at least one chemical entity exhibits a IC₅₀ value less than or equal to 25 micromolar.

59. At least one chemical entity according to claim 1, wherein in an in vitro assay of modulation of cell growth in soft agar, the at least one chemical entity exhibits an IC₅₀ value less than or equal to 25 micromolar, wherein the cells are chosen from HCT-15, MiaPaca-2, MCF-7, OVCAR-4, and A549 cells, the at least one chemical entity exhibits an IC₅₀ value less than or equal to 25 micromolar.

60. A pharmaceutical composition comprising at least one chemical entity according to claim 1; and at least one pharmaceutically acceptable carrier or excipient.

61. A method of treating a kinase-implicated condition in a mammal having a kinase-implicated condition, comprising administering to the mammal a therapeutically effective amount of at least one chemical entity of claim 1.

62. The method of claim 61, wherein the mammal is a human.

63. The method of claim 61, wherein the mammal is chosen from cats and dogs.

64. A method of treating cancer, comprising administering to a mammal in need thereof a therapeutically effective amount of at least one chemical entity according to claim 1.

65. A method of treating cancer, comprising administering to a mammal in need thereof a therapeutically effective amount of at least one chemical entity according to claim 1 and a therapeutically effective amount of at least one antitumor therapeutic.

66. The method of claim 65, wherein treatment with the at least one antitumor therapeutic follows treatment with the at least one chemical entity of claim 1.

67. The method of claim 65, wherein the at least one antitumor therapeutic is at least one other chemotherapeutic agent.

68. The method of claim 67, wherein the at least one chemotherapeutic agent is chosen from mitomycin C, carboplatin, taxol, cisplatin, paclitaxel, etoposide, and doxorubicin.

69. The method of claim 65, wherein the at least one antitumor therapeutic is at least one radiotherapeutic agent.

70. The method of claim 65, wherein the mammal is a human.

71. The method of claim 65, wherein the mammal is chosen from cats and dogs.

72. A method for identifying a kinase, comprising contacting an organism, cell, or preparation comprising the kinase with at least one chemical entity according to claim 1, and detecting modulation of an activity of a kinase, whereby the kinase is identified.

73. A method of treating a Btk-implicated condition in a mammal, comprising administering to the mammal a therapeutically effective amount of at least one chemical entity according to claim 1.

74. The method of claim 73 wherein an additional kinase is implicated in the condition.

75. The method of claim 73 wherein the mammal is a human.

76. The method of claim 73 wherein the mammal is chosen from cats and dogs.

77. A method for identifying Btk, comprising
contacting an organism, cell, or preparation comprising the kinase with at least one chemical entity according to claim 1, whereby
detecting modulation of an activity of Btk, whereby Btk is identified.

78. A method of treating a Btk-implicated autoimmune/inflammatory condition in a mammal, comprising administering to the mammal a therapeutically effective amount of at least one chemical entity according to claim 1.

79. The method of claim 78, wherein the mammal is a human.

80. The method of claim 78 wherein the mammal is chosen from cats and dogs.

81-83. (canceled)


SEQUENCE LISTING (10 sequences)

SEQ ID NO: 1 - protein, 16 aa, Artificial Sequence
  Biotinylated Crosstide peptide
KGSGSGRPRTSSFAEG

SEQ ID NO: 2 - DNA, 30 bp, Artificial Sequence
  Probe
ccgctcgagactgaagtggaccccacacat

SEQ ID NO: 3 - DNA, 30 bp, Artificial Sequence
  Probe
cgggntaccttattttaaaagtgcttcaaa

SEQ ID NO: 4 - DNA, 31 bp, Artificial Sequence
  Probe
acgcgtcgacggtgcctttgaagaccgggat

SEQ ID NO: 5 - DNA, 34 bp, Artificial Sequence
  Probe
atagtttagcggccgctcagaatgaaggtcattt

SEQ ID NO: 6 - DNA, 30 bp, Artificial Sequence
  Probe
ccgctcgagtatgcctgccaagaccccacg

SEQ ID NO: 7 - DNA, 30 bp, Artificial Sequence
  Probe
cggggtaccctatgaaaaggacagggagtg

SEQ ID NO: 8 - DNA, 33 bp, Artificial Sequence
  Probe
ggagcactcgagatggtagcacacaaccaggtg

SEQ ID NO: 9 - DNA, 35 bp, Artificial Sequence
  Probe
ggagcaggaattcnggcgctgcnggtcaaatctgg

SEQ ID NO: 10 - protein, 17 aa, Artificial Sequence
  Biotinylated substrate
EGPWLEEEEEAYGWMDF